(19) United States
(12) Patent Application Publication
(10) Pub. No.: US 2023/0272434 A1
(43) Pub. Date: Aug. 31, 2023

(54) GENOMIC EDITING WITH SITE-SPECIFIC RETROTRANSPOSONS

(71) Applicant: Massachusetts Institute of Technology, Cambridge, MA (US)

(72) Inventors: Omar Abudayyeh, Cambridge, MA (US); Jonathan Gootenberg, Cambridge, MA (US); Lukas Villiger, Cambridge, MA (US); Justin Lim, Boston, MA (US)

(21) Appl. No.: 18/047,685

(22) Filed: Oct. 19, 2022

Related U.S. Application Data
(60) Provisional application No. 63/262,714, filed on Oct. 19, 2021.
(60) Provisional application No. 63/371,246, filed on Aug. 12, 2022.

(51) Int. Cl. C12N 15/90; C12N 9/22; C12N 15/10; C12N 15/113; C12N 15/63

(52) U.S. Cl. CPC C12N 15/907; C12N 9/22; C12N 15/102; C12N 15/113; C12N 15/63; C12N 2800/80; C07K 2319/80

(57) ABSTRACT

Genome editing tools for use in systems designed to deliver large genetic elements are disclosed herein. A genome editing system is described, which includes i) an R2 element enzyme or other non-LTR site specific retrotransposon element and ii) a payload RNA, wherein the payload RNA comprises an insertion region and optionally one or more of a 5′ homology region, a 3′ homology region, and a protein binding element, wherein the insertion region comprises a template for a small or large nucleic acid insertion into the genome, and wherein the R2 element enzyme or other non-LTR site specific retrotransposon element comprises a targeting domain, a reverse transcriptase domain, and a nickase domain. Also disclosed are cells edited using such a genome editing system, methods for editing a genome, and compositions comprising cells edited with this genomic editing system.

### RELATED APPLICATION

This application claims the benefit of U.S. Provisional Patent Application Ser. Nos. 63/262,714 and 63/371,246 respectively filed on Oct. 19, 2021, and Aug. 12, 2022, and the entire disclosure of which is incorporated herein by reference.

### STATEMENT AS TO FEDERALLY FUNDED RESEARCH

This invention was made with Government support under Grant No. R21 AI149694 awarded by the National Institutes of Health (NIH) and under Grant No. R01 EB031957. The Government has certain rights in this invention.

### SEQUENCE LISTING

The instant application contains a Sequence Listing which has been submitted electronically in XML file format and is hereby incorporated by reference in its entirety. Said XML, copy, created on Oct. 18, 2022, is named 733339_083474-024_SL.xml and is 66,696,250 bytes in size.

### BACKGROUND

Genome editing systems have developed as a promising technology for the development of therapeutic tools. Systems such as CRISPR/Cas9, TALEN, and zinc finger proteins have been used to alter the genomes of organisms. However, these systems are limited by a number of factors, including size, cargo capacity, and targeting ability.

Retrotransposons are mobile elements that insert themselves into the genome of a host through an RNA intermediate. This is in contrast to the mechanism of most DNA transposons, which directly insert themselves into a host genome. Retrotransposons are categorized as long terminal repeat (LTR) retrotransposons and non-LTR retrotransposons.

Non-LTR retrotransposons are among the most frequently occurring transposable elements in the eukaryotic genome. They can be either randomly inserting or site-specific. Site-specific non-LTR retrotransposons are generally characterized by the presence of specific activity—reverse transcriptase activity, DNA nicking activity, and nucleic acid binding activity. The genetic loci for these activities are found in either a single open reading frame (ORF) or split between two ORFs. The DNA nicking activity of single-ORF systems is found with restriction-like endonuclease (RLE) domains. Multiple non-LTR retrotransposon families, such as the R2, R4, R5, R8, R9, Dong and Cre families, are categorized as RLE containing non-LTR retrotransposons.

Of the known non-LTR retrotransposons, the most well studied is the R2 element. The R2 element is comprised of R2 RNA and the R2 protein. The R2 element contains a single open reading frame (ORF), which encodes a reverse transcriptase, an endonuclease, and includes DNA binding regions and zinc finger motifs. R2 element. R2 inserts itself into a host genome through a mechanism known as Target Primed Reverse Transcription (TPRT), which is a stepwise reaction including a first nick of host DNA, reverse transcription of the R2 RNA into the first strand, a second nick of host DNA, and synthesis of a second strand.

The mechanism by which the R2 element inserts into a host genome, being independent of endogenous cellular repair pathways, as well as the capacity to carry an RNA molecule of varying sizes to a host genome, makes the R2 element a potentially powerful genome editing system. However, the R2 element specifically inserts itself into either the 28S or 18S ribosomal RNA locus. Therefore, it lacks the ability to target insertions to a particular locus, which is a critical aspect for viable genome editing systems. Other site-specific retrotransposons are similarly limited to particular loci. There remains an unmet need for a genome editing system that is capable of directed insertion of large nucleic acids into a host genome.

### BRIEF SUMMARY

The present disclosure is directed to a genome editing system comprising: i) an R2 element enzyme; and ii) a payload RNA, wherein the payload RNA comprises an insertion template and optionally one or more of a 5′ homology region, a 3′ homology region, and a protein binding element, wherein the insertion template comprises a sequence for a nucleic acid insertion into the genome, and wherein the R2 element enzyme comprises a reverse transcriptase domain, and a nickase domain.

In some embodiments the R2 element enzyme further comprises a targeting domain. In some embodiments the targeting domain is a natural targeting domain or an engineered targeting domain. In some embodiments, the nucleic acid insertion into the genome is a DNA or RNA insertion template. In some embodiments, the R2 element enzyme is a modified R2 element enzyme. In some embodiments, the coding sequence of the R2 element enzyme is modified. In some embodiments, wherein the modified R2 element enzyme is modified by an N-terminal or C-terminal truncation of the R2 element enzyme sequence. In some embodiments, the modified R2 element enzyme comprises a linker. In some embodiments the linker is an XTEN linker.

In some embodiments, the genome editing system targets a genomic locus. In some embodiments, the genome editing system targets a genomic locus other than the 28S rRNA locus. In some embodiments, an N-terminal zinc finger domain of the R2 element enzyme is modified to target a genomic locus other than the 28S rRNA locus. In some embodiments, a non-naturally occurring targeting region is fused to the N-terminus of the R2 element enzyme or inserted into the R2 element enzyme.

In some embodiments, the modified R2 element enzyme is a fusion protein. In some embodiments, the modified R2 element is fused to a Cas9 protein that is fully active, catalytically dead (H840A/D10A for SpCas9), or functioning as a nickase (H840A or D10A for SpCas9). In some embodiments, the modified R2 element is fused to a Cas12 protein that is fully active, catalytically dead, or functioning as a nickase. In some embodiments, the modified R2 element is fused to a TALEN protein, zinc finger protein, argonaute, or meganuclease protein.

In some embodiments, the genome editing system further comprises a guide RNA. In some embodiments, the 5′ homology region of the payload RNA is engineered to target a genomic locus other than the 28S rRNA locus. In some embodiments, the 5′ homology region, the 3′ homology region, or both the 5′ and 3′ homology region target an exogenously introduced landing sequence.

In some embodiments, the insertion region is introduced into the genome of a specific cell type. In some embodiments, the specific cell type is a post-mitotic cell. In some embodiments, the genome editing system functions in post-mitotic cells. In some embodiments, the genome editing system functions independently from intrinsic nucleic acid repair systems.

In some embodiments, the payload RNA template further comprises a 5′ untranslated region (UTR), a 3′ UTR, or both a 5′ UTR and a 3′ UTR. In some embodiments, the 5′ homology region and the 3′ homology region are located between the 5′ UTR and 3′ UTR. In some embodiments, the 5′ homology region and the 3′ homology region are located outside the 5′ UTR and 3′ UTR. In some embodiments, the payload RNA further comprises a 5′ untranslated region (UTR), a 3′ UTR, or both a 5′ and a 3′ UTR, wherein the UTRs are truncated. In some embodiments, the payload RNA does not comprise a 5′ UTR. In some embodiments, the payload RNA does not comprise a 3′ UTR.

In some embodiments, the payload RNA further comprises a nuclear retention element. In some embodiments, the payload RNA further comprises a Cas9 or Cas12 guide RNA, wherein the Cas9 or Cas12 guide RNA comprises an extension with a 5′ homology sequence, a 3′ homology sequence, a 5′ untranslated region (UTR), a 3′ UTR, an insertion template, or any combination thereof. In some embodiments the nucleic acid insertion template is a sequence of greater than 1000 base pairs.

In some embodiments, the R2 element enzyme comprises a nuclear localization signal (NLS).

In some embodiments, the insertion region comprises a template for a reporter gene, a transcription factor gene, a transgene, an enzyme gene, or a therapeutic gene.

The present disclosure is also directed to a method of inserting a large nucleic acid into a genome within a cell using a Cas9 or Cas12 fusion protein, wherein the method comprises supplying a Cas9 or Cas12 fusion protein to a cell, wherein the Cas9 or Cas12 fusion protein is supplied with a payload RNA template, wherein the RNA template is reverse transcribed by the Cas9 or Cas12 fusion protein prior to being inserted into the genome of the cell; and wherein the large nucleic acid is inserted into the genome of the cell.

In some embodiments, the Cas9 fusion protein comprises a Cas9 portion and an R2 element portion. In some embodiments, the Cas9 fusion protein comprises a targeting domain, a reverse transcriptase domain, and a nickase domain. In some embodiments, the Cas12 fusion protein comprises a Cas12 portion and an R2 element portion.

The disclosure is also directed to a method of inserting an exogenous nucleic acid into the genome of a post-mitotic cell, wherein the method comprises subjecting the genome of the post-mitotic cell to a modified Cas9 protein that inserts the exogenous nucleic acid into the genome of the post-mitotic cell. In some embodiments, the modified Cas9 protein is fused to an R2 element enzyme. In some embodiments, the modified Cas9 fusion protein targets an endogenous landing site. In some embodiments, the Cas9 fusion protein targets an exogenously introduced landing site in the genome of the post-mitotic cell.

The disclosure is also directed to a method of editing a genome comprising subjecting the cell to the genome editing systems described above.

The disclosure is also directed to a composition comprising a cell edited by the genome editing systems or methods of editing genomes described above.

The disclosure is also directed to a genome editing system comprising: i) a payload RNA, wherein the payload RNA comprises an insertion template and optionally one or more of a 5′ homology region, a 3′ homology region, and a protein binding element, wherein the insertion template comprises a sequence for a nucleic acid insertion into the genome; ii) a non-LTR site specific retrotransposon element enzyme; wherein the non-LTR site specific retrotransposon element enzyme comprises a reverse transcriptase domain and, optionally, a nuclease or nickase domain, and wherein if the non-LTR-site specific retrotransposon element enzyme does not comprise the optional nuclease or nickase domain, the genome editing system further comprises iii) a nuclease or nickase enzyme. In some embodiments, the nuclease or nickase enzyme is a programmable nuclease or nickase. In some embodiments, the non-LTR site specific retrotransposon element enzyme further comprises a targeting domain. In some embodiments, the targeting domain is a natural targeting domain or an engineered targeting domain.

The disclosure is also directed to a genome editing system where the non-LTR site specific retrotransposon comes from the R1, R2, R4, R5, R6, R7, R8, R9, CRE, NeSL, HERO, or Utopia families, or from the 9 family classifications established for RLE domain containing nLTR retrotransposons (FIG. 24C).

In some embodiments, the nucleic acid insertion into the genome is a DNA or RNA insertion template.

In some embodiments, the non-LTR site specific retrotransposon element enzyme is a modified non-LTR site specific retrotransposon element enzyme. In some embodiments, the coding sequence of the non-LTR site specific retrotransposon element enzyme is modified. In some embodiments, the modified non-LTR site specific retrotransposon element enzyme is modified by an N-terminal or C-terminal truncation of the non-LTR site specific retrotransposon element enzyme sequence.

In some embodiments, the modified non-LTR site specific retrotransposon element enzyme comprises a linker. In some embodiments, the linker is an XTEN linker.

The genome editing system of the disclosure targets a genomic locus. In some embodiments, the genome editing system targets a genomic locus other than the 28S rRNA locus. In some embodiments, an N-terminal zinc finger domain of the non-LTR site specific retrotransposon element enzyme is modified to target a genomic locus other than the 28S rRNA locus. In some embodiments, a non-naturally occurring targeting region is fused to the N-terminus of the non-LTR site specific retrotransposon element enzyme or inserted into the non-LTR site specific retrotransposon element enzyme.

In some embodiments, the modified non-LTR site specific retrotransposon element enzyme is a fusion protein. In some embodiments, the modified non-LTR site specific retrotransposon element is fused to a Cas9 protein that is fully active, catalytically dead (H840A/D10A for SpCas9), or functioning as a nickase (H840A or D10A for SpCas9). In some embodiments, the modified non-LTR site specific retrotransposon element is co-delivered with a Cas9 protein that is fully active, catalytically dead (H840A/D10A for SpCas9), or functioning as a nickase (H840A or D10A for SpCas9). In some embodiments, the modified non-LTR site specific retrotransposon element is fused to a Cas12, IscB, IsrB, or TnpB protein that is fully active, catalytically dead, or functioning as a nickase. In some embodiments, the modified non-LTR site specific retrotransposon element is delivered in trans with a Cas12, IscB, IsrB, or TnpB protein that is fully active, catalytically dead, or functioning as a nickase. In some embodiments, the modified non-LTR site specific retrotransposon element is fused to a TALEN protein, zinc finger protein, argonaute, or meganuclease protein.

In some embodiments, the disclosure further comprises a guide RNA. In some embodiments, the disclosure further comprises multiple guide RNA.

In some embodiments, the genome editing system of the disclosure comprises a payload wherein the 5′ homology region, the 3′ homology region, or both the 5′ and 3′ homology region of the payload RNA is engineered to target a genomic locus other than the 28S rRNA locus. In some embodiments, the 5′ homology region, the 3′ homology region, or both the 5′ and 3′ homology region target an exogenously introduced landing sequence.

In some embodiments, the insertion region is introduced into the genome of a specific cell type. In some embodiments, the specific cell type is a post-mitotic cell, a non-dividing cell, or a quiescent cell. In some embodiments, the genome editing system functions in post-mitotic cells, non-dividing cells, or quiescent cells. In some embodiments, the genome editing system functions independently from intrinsic nucleic acid repair systems.

In some embodiments, the payload RNA template further comprises a 5′ untranslated region (UTR), a 3′ UTR, or both a 5′ UTR and a 3′ UTR. In some embodiments, the 5′ homology region and the 3′ homology region are located between the 5′ UTR and 3′ UTR. In some embodiments, the 5′ homology region and the 3′ homology region are located outside the 5′ UTR and 3′ UTR. In some embodiments, the payload RNA further comprises a 5′ untranslated region (UTR), a 3′ UTR, or both a 5′ and a 3′ UTR, wherein the UTRs are truncated. In some embodiments, the payload RNA does not comprise a 5′ UTR. In some embodiments, the payload RNA does not comprise a 3′ UTR. In some embodiments, the payload RNA further comprises a nuclear retention element. In some embodiments, the payload RNA further comprises a Cas9 or Cas12 guide RNA, and wherein the Cas9 or Cas12 guide RNA comprises an extension with a 5′ homology sequence, a 3′ homology sequence, a 5′ untranslated region (UTR), a 3′ UTR, an insertion template, or any combination thereof.

In some embodiments, the nucleic acid insertion template is a sequence of greater than 1000 base pairs.

In some embodiments, the genome editing system targets a genome for a deletion. In some embodiments, the deletions are between 1 and 150 bases.

In some embodiments, the non-LTR site specific retrotransposon element enzyme comprises a nuclear localization signal (NLS).

In some embodiments, the insertion region comprises a template for a reporter gene, a transcription factor gene, a transgene, an enzyme gene, or a therapeutic gene.

The disclosure is also directed to a method of inserting a large nucleic acid into a genome within a cell using a Cas9 or Cas12 fusion protein, wherein the method comprises supplying a Cas9 or Cas12 fusion protein to a cell, wherein the Cas9 or Cas12 fusion protein is supplied with a payload RNA template, wherein the RNA template is reverse transcribed by the Cas9 or Cas12 fusion protein prior to being inserted into the genome of the cell; and wherein the large nucleic acid is inserted into the genome of the cell. In some embodiments, the Cas9 fusion protein comprises a Cas9 portion and a non-LTR site specific retrotransposon element portion. In some embodiments. the Cas9 fusion protein comprises a targeting domain, a reverse transcriptase domain, and a nickase domain. In some embodiments, the Cas12 fusion protein comprises a Cas12 portion and a non-LTR site specific retrotransposon element portion.

The disclosure is also directed to a method of inserting an exogenous nucleic acid into the genome of a post-mitotic cell, wherein the method comprises subjecting the genome of the post-mitotic cell to a modified Cas9 protein that inserts the exogenous nucleic acid into the genome of the post-mitotic cell. In some embodiments, the modified Cas9 protein is fused to a non-LTR site specific retrotransposon element enzyme. In some embodiments, the modified Cas9 fusion protein targets an endogenous landing site. In some embodiments, the Cas9 fusion protein targets an exogenously introduced landing site in the genome of the post-mitotic cell.

The disclosure is also directed to a method of editing a genome comprising subjecting the cell to the genome editing system as described herein. The disclosure is also directed to a composition comprising the cell edited by the genome editing methods described herein.

The disclosure is also directed to a method of correcting a genetic mutation related to disease or human pathology, wherein the method comprises making small nucleotide changes or small nucleotide insertions (1-100 bp) in a human genome using the genome editing system of claim 1 or claim 47.

In some embodiments, the genome editing system is delivered via single or multi vector AAV, adenovirus, lentivirus, herpes simplex virus, PEG10 viral like particles, PNMA viral like particles, gag-like viral like particles, nanoblades, gesicles, or Friend murine leukemia virus (FMLV) viral like proteins.

In some embodiments, the components of the genome editing system are delivered as all RNA in lipid nanoparticles or another RNA delivery reagent. In some embodiments, wherein the non-LTR site specific retrotransposon is delivered as mRNA. In some embodiments, the guide RNAs are delivered as synthetic RNA. In some embodiments, the payload is delivered as mRNA.

The disclosure is also directed to a genome editing system targets and edits the genome at more than one site.

### BRIEF DESCRIPTION OF THE DRAWINGS

FIG. 1 is a visual depiction of PCR products isolated on an agarose gel following amplification from isolated DNA from HEK293FT cells which were transfected with two plasmids, showing insertion of R2 into the human genome. Lane 1 displays a molecular weight marker. Lane 2 displays PCR products from cells transfected with an R2 plasmid, encoding an R2 derived from the zebra finch (Taeniopygia guttata) R2 element (R2Tg) with an eGFP payload. Lane 3 displays the PCR products from cells transfected with R2Tg alone. Lane 4 displays the PCR products from cells transfected with eGFP payload alone. Lanes 5 and 6 display the PCR products from cells transfected with R2 orthologs from Geospiza fortis (Gfo) and a long Gfo payload (Lane 5) or short Gfo payload (Lane 6). Lane 7 displays PCR product from cells transfected with an R2 ortholog from Geospiza fortis alone. Lane 8 displays PCR product from cells transfected with only long Gfo payload. Lane 9 displays PCR product from cells transfected with only short Gfo payload.

FIG. 2 is a graphical depiction of luminescence readout from HEK293FT cells transfected with 3 separate plasmids: the first containing an R2 protein encoding region, the second containing an inactive luciferase reporter region (containing the promoter region and a first of two artificial and inactive luciferase exons followed by a chimeric intron) with R2 landing sites (the landing site is placed in an intronic region that is spliced out after insertion of the payload carrying the second of two artificial exons) of variable length, and the third containing a luciferase portion of a payload, 5′ and 3′ UTRs as well as regions homologous to the landing sites. The x-axis labels represent variable landing sites, named according to the number of base pairs (bp) present on the landing site on either side of the insertion; 38/10 therefore, represents 38 bp upstream of the insertion site and 10 bp downstream of the insertion site. Columns 11 and 12 display the luminescence readout of two negative controls, AAVS1_target (non-target) and CFTR_target (non-target).

FIGS. 3A and 3B are graphical depictions of the tolerability of mutations of the landing sites with respect to R2 integration in HEK293FT cells. HEK293FT cells were transfected with 3 separate plasmids: the first containing an R2 protein encoding region, the second containing a luciferase reporter region with mutated or wild type R2 (28S) landing sites in the intronic region that follows the first of two luciferase exons, and the third (payload) plasmid containing the second exon necessary for luciferase signal after insertion and splicing of the reporter plasmid. Additionally to a small intronic sequence upstream of the second of two artificial luciferase exons (minimal cargo necessary for luciferase signal), the payload plasmid has 5′ and 3′ UTR sequences as well as 5′ and 3′ homologies (100 bp homology to the 28S locus on either side). FIG. 3A displays the location of certain mutations within the region flanking the insertion on the insertion region plasmid. Figure discloses SEQ ID NOS 33523-33534, respectively, in order of appearance. FIG. 3B is a readout of luminescence from HEK293FT cells transfected as above. The y-axis represents the specific plasmids containing altered landing sites introduced into the specific cell, with each name representing the number of base pairs (bp) present on the landing site on either side of the insertion; 37/23 therefore, represents 37 bp upstream of the insertion site and 23 bp downstream of the insertion site. A 115/115 negative control (transfected cell with no plasmid expressing R2).

FIGS. 4A and 4B are graphical depictions of the tolerability of mutations of landing sites with respect to R2 integration in HEK293FT cells. HEK293FT cells were transfected with 3 separate plasmids: the first containing an R2 protein encoding region, the second containing a luciferase reporter region with mutated or wild type R2 landing sites and the third (payload) plasmid containing the second exon necessary for luciferase signal after insertion and splicing of the reporter plasmid. Additionally to a small intronic sequence upstream of the second of two artificial luciferase exons (minimal cargo necessary for luciferase signal), the payload plasmid has 5′ and 3′ UTR sequences as well as 5′ and 3′ homologies (100 bp homology to the 28S locus on either side). FIG. 4A displays the location of certain mutations within the region flanking the insertion on the insertion region plasmid. Figure discloses SEQ ID NOS 33535-33546, respectively, in order of appearance. FIG. 4B is a readout of luminescence from HEK293FT cells transfected as above. Target_37_23_mut_10 (red box) has full mutations of all three, predicted zinc finger binding sites.

FIG. 5 is a graphical depiction of the effect of aphidicolin on the integration of a luciferase payload into a target region. HEK293FT cells were transfected with 3 separate plasmids: the first containing an R2 protein encoding region, the second containing a luciferase reporter region with R2 landing sites, and the third (payload) plasmid containing the second exon necessary for luciferase signal after insertion and splicing of the reporter plasmid. Additionally to a small intronic sequence upstream of the second of two artificial luciferase exons (minimal cargo necessary for luciferase signal), the payload plasmid has 5′ and 3′ UTR sequences as well as 5′ and 3′ homologies (100 bp homology to the 28S locus on either side) Cells were then treated] with either Dimethyl Sulfoxide (DMSO) or aphidicolin at a concentration of 1 μm, 5 μm, or 25 μm. Homologous sequences in the insertion region were either 60 bp or 40 bp long. Columns 9-12 are cells treated with either DMSO or aphidicolin and transfected with negative control plasmids.

FIG. 6 is a graphical depiction of the effect of aphidicolin on the integration of a luciferase payload into a target region. HEK293FT cells were transfected with 3 separate plasmids: the first containing an R2 protein encoding region, the second containing a luciferase reporter region with R2 landing sites, and the third (payload) plasmid containing the second exon necessary for luciferase signal after insertion and splicing of the reporter plasmid. Additionally to a small intronic sequence upstream of the second of two artificial luciferase exons (minimal cargo necessary for luciferase signal), the payload plasmid has 5′ and 3′ UTR sequences as well as 5′ and 3′ homologies (100 bp homology to the 28S locus on either side. Cells were then treated with either Dimethyl Sulfoxide (DMSO) or aphidicolin at a concentration of 1 μm, 5 μm, or 25 μm. The insertion regions of the plasmids are flanked by either 300 bp, 200 bp, or 100 bp. Columns 13-16 contain a 300 bp flanking sequence in the insertion region and were simultaneously transfected with a plasmid without an active R2 enzyme. Columns 17-20 were solely transfected with a Cas9 plasmid.

FIG. 7 is a visual depiction of a heatmap showing the luminescence readout of HEK293FT cells transfected with 3 separate plasmids. The first plasmid contained an R2 protein encoding region, the second plasmid contained a luciferase reporter precursor region with R2 landing sites, and the third (payload) plasmid containing the second exon necessary for luciferase signal after insertion and splicing of the reporter plasmid. Additionally to a small intronic sequence upstream of the second of two artificial luciferase exons (minimal cargo necessary for luciferase signal), the payload plasmid has 5′ and 3′ UTR sequences as well as 5′ and 3′ homologies of different length (from 0 to 100 bp homology in steps of 20 bp).

FIG. 8 is a graphical depiction of the effect of modification of UTRs on the luminescence readout of transfected HEK392FT cells. HEK293FT cells were transfected with 3 separate plasmids. The first plasmid contained an R2 protein encoding region, the second plasmid contained a splice luciferase reporter region with R2 landing sites 26/22 bp, and the third (payload) plasmid containing the second exon necessary for luciferase signal after insertion and splicing of the reporter plasmid. Additionally to a small intronic sequence upstream of the second of two artificial luciferase exons (minimal cargo necessary for luciferase signal), the payload plasmid has 5′ and 3′ UTR sequences that are truncated in different ways as well as 5′ and 3′ homologies. Column 1 represents a positive control. Column 2 represents a negative control. Columns 3-8 represent truncations from the left of the 5′UTR. Columns 9-15 represent truncations from the right of the 5′ UTR. Columns 16-22 represent truncations from the left of the 3′ UTR. Columns 23-29 represent truncations from the right of the 3′UTR.

FIG. 9A is a graphical depiction exhibiting the effect that altered homology regions have on integration. HEK293FT cells were transfected with 3 separate plasmids: the first containing an R2 protein encoding region, the second containing a luciferase reporter region with 26/22 bp R2 landing sites and the third (payload) plasmid containing the second exon necessary for luciferase signal after insertion and splicing of the reporter plasmid. Additionally to a small intronic sequence upstream of the second of two artificial luciferase exons (minimal cargo necessary for luciferase signal), the payload plasmid has 5′ and 3′ UTR sequences as well as 5′ and 3′ homologies. Here, the 3′ homologies have different lengths: PBS13 (13 bp) and 3′ homology (100 bp). HDV is an HDV ribozyme, which cleaves the insertion region directly after the 3′ UTR and mHDV is a mutated HDV ribozyme that is non-functional. FIG. 9B is a visual representation of each 3′ modification.

FIG. 10 is a graphical depiction of the effect of linker insertion site on integration efficiency of the R2 protein. Linkers were inserted into various domains at specific insertion sites of an R2 derived from the zebra finch (Taeniopygia guttata) R2 element (R2Tg) with an eGFP or msfGFP payload. Positions for linkers were identified using Emboss gamier to identify potential linker regions, of which 12 were chosen. Linkers for eGFP, for example, were GSGGGSGS (SEQ ID NO: 33377)-EGFP-GSGGGGSG (SEQ ID NO: 33378). Columns 1 and 2 are wild-type R2Tg without a linker region.

FIG. 11 is a graphical depiction of editing efficiency in the short 28S landing site in an exogenous plasmid. HEK293FT cells were transfected with 3 separate plasmids: the first either containing an R2 protein encoding region or no R2 protein encoding region, the second containing a luciferase reporter region with 26/22 (26 upstream/22 downstream) R2 landing sites and the third (payload) plasmid containing the second exon necessary for luciferase signal after insertion and splicing of the reporter plasmid. Additionally, to a small intronic sequence upstream of the second of two artificial luciferase exons (minimal cargo necessary for luciferase signal), the payload plasmid has 5′ and 3′ UTR sequences and 100 bp 5′ and 3′ homologies to the 28S target site. Percent editing is measured by digital droplet PCR (ddPCR) using primers that recognize the payload.

FIG. 12 is a graphical depiction of R2 insertion efficiency within the endogenous Beta actin locus of HEK293FT cells transfected with 4 separate plasmids: the first containing an R2 protein encoding region, the second containing an insertion region with a pMAX gene flanked by 5′ and 3′ UTRs and homology regions to the 28S locus, the third a prime editor encoding region, and the fourth a prime editing guideRNA to introduce a 26/22 R2 target site at the ACTB locus. From left to right, the samples are 1) wild-type R2 protein, 2) R2 protein fused to a nuclear localization signal, 3) no R2 protein with Prime editing molecule, 4) R2 protein without prime editing molecule. Percent integration is measured by ddPCR.

FIG. 13A is a visual depiction of the integration a payload comprised of an R2 protein attached at the C-terminus to eGFP. FIG. 13B is graphical depiction is a luminescence readout of the effect of addition of a nuclear localization signal to the N and C-terminus of the R2 protein on reporter expression. Either wild-type R2 (column 1) or NLS-appended R2 (column 2) were transfected into HEK293FT cells with a stably integrated splice reporter. A negative control is shown in column 3.

FIGS. 14A-D are visual depictions of HEK293FT cells transfected with either an R2 expression plasmid (FIGS. 14A, 14B) or an R2 negative plasmid (FIGS. 14C, 14D) at either 20 hours post transfection (FIGS. 14A, 14C) or 36 hours post transfection (FIGS. 14B, 14D). The R2 template inserts a second GFP exon into the stably transfected splice receptor, which contains the promoter and a first exon, allowing for GFP expression following integration.

FIGS. 15A and 15B are graphical depictions of the percentage of GFP positive cells as determined by flow cytometry following transfection of specific plasmids. FIG. 15A is a graph depicting fluorescent readout of cells transfected with plasmids with wild-type R2 (column 1), a negative control (no R2 protein; column 2), 300 ng of R2 with a nuclear localization signal (column 3), 200 ng of R2 with a nuclear localization signal (column 4), 100 ng of R2 with a nuclear localization signal (column 5), 50 ng of R2 with a nuclear localization signal (column 5), and untransfected cells as a percentage of all cells in each sample. FIG. 15B is a graph depicting fluorescent readout of cells transfected with plasmids with wild-type R2 (column 1), a negative control (no R2 protein; column 2), 300 ng of R2 with a nuclear localization signal (column 3), 200 ng of R2 with a nuclear localization signal (column 4), 100 ng of R2 with a nuclear localization signal (column 5), 50 ng of R2 with a nuclear localization signal (column 5), and untransfected cells as a percentage of the number of transfected cells in each sample.

FIG. 16A is a graphic depiction exhibiting the effect that N-terminal truncations of the R2 protein have on integration. HEK293FT cells were transfected with 3 separate plasmids: the first containing an R2 protein encoding region, in which the R2 protein has been truncated from the N-terminus, the second containing a luciferase reporter region with 26/22 bp R2 landing sites, and the third (payload) plasmid containing the second exon necessary for luciferase signal after insertion and splicing of the reporter plasmid. Additionally to a small intronic sequence upstream of the second of two artificial luciferase exons (minimal cargo necessary for luciferase signal), the payload plasmid has 5′ and 3′ UTR and 5′ and 3′ homologies to the 28S target site. Wild-type R2 (column 1) and negative control (column 2) are also depicted. FIG. 16B is a visual representation of the N-terminal truncations of the R2 protein. Each horizontal bar represents the R2 protein expressed, with further N-terminal regions being removed as the numbers go from 1 to 10.

FIG. 17A is a graphic depiction exhibiting the effect that C-terminal truncations of the R2 protein have on integration. HEK293FT cells were transfected with 3 separate plasmids: the first containing an R2 protein encoding region, in which the R2 protein has been truncated from the C-terminus, the second containing a luciferase reporter region with 26/22 bp R2 landing sites, and a third (payload) plasmid containing the second exon necessary for luciferase signal after insertion and splicing of the reporter plasmid. Additionally to a small intronic sequence upstream of the second of two artificial luciferase exons (minimal cargo necessary for luciferase signal), the payload plasmid has 5′ and 3′ UTR sequences and 100 bp 5′ and 3′ homologies to the 28S target site. Wild-type R2 (column 1) and negative control (column 2) are also depicted. FIG. 17B is a visual representation of the N-terminal truncations (Nt_1-Nt_10 from FIG. 16) as well as the C-terminal truncations (Ct_1-Ct_6) of the R2 protein. Each horizontal bar represents the R2 protein expressed, with further N or C-terminal regions being removed as the numbers get larger.

FIG. 18 is a graphical representation of the luminescence readout of HEK293FT cells transfected with three separate plasmids. HEK293FT cells were transfected with 3 separate plasmids. The first plasmid either contained an R2 protein encoding region, no R2 protein encoding region, or an R2 protein with a catalytically inactive restriction-like endonuclease (RLE) domain, which should ablate insertion activity. The second plasmid contained a luciferase reporter region with 26/22 (26 upstream/22 downstream) R2 landing sites, and the third (payload) plasmid contained the second artificial exon necessary for luciferase signal after insertion and splicing of the reporter plasmid. Additionally to a small intronic sequence upstream of the second of two artificial luciferase exons (minimal cargo necessary for luciferase signal), the payload plasmid has 5′ and 3′ UTR sequences as well as 5′ and 3′ homologies.

FIG. 19 is a graphical representation of the luminescence readout of HEK293FT cells transfected with three separate plasmids. HEK293FT cells were transfected with 3 separate plasmids. The first plasmid either contained an R2 protein encoding region, no R2 protein encoding region, or an R2 protein lacking one of several specific R2 protein domains. The second plasmid contained a luciferase reporter region with 26/22 (26 upstream/22 downstream) R2 landing sites. The third plasmid contained an insertion region with a luciferase insertion as well as modified or unmodified UTRs. Columns 1-3 display the results when the transfected R2 protein is an R2 protein in which the −1 domain, which is an RNA interaction domain, has been deleted. Columns 4-6 display the results when the transfected R2 protein is an R2 protein in which the −1 and the 0 domain, which is also an RNA interaction domain, has been deleted. Columns 7-9 display the results when the transfected R2 protein is an R2 protein in which the 0 domain has been deleted. Columns 10-12 display the results when the transfected R2 protein is an R2 protein in which the 0 domain has been replaced by an eGFP domain. Columns 13-15 display the results when the transfected R2 protein is an R2 protein in which the 0 domain has been replaced by an MS2 coat protein (MCP) domain, which binds to MS2 binding sites. Columns 16-18 display the results when the transfected R2 protein is an R2 protein with the N-terminal 6_2 truncation, and the MCP domain has been fused to the new N-terminus. Columns 19-21 display the results when the transfected R2 protein is an R2 protein with the N-terminal 6_2 truncation, MCP domain fused to the new N-terminus, and the zinc finger domain has been deleted. Columns 22-24 display the results when the transfected R2 protein includes a c-terminal MCP fusion. Columns 25-27 display wild-type R2, and columns 28-30 display the negative control. Orange bars have a payload which includes a wild-type luciferase with 5′ and 3′ UTRs. Blue bars indicate payloads in which the 5′ UTR is replaced by extended MS2 regions. Green bars indicate payloads in which both the 5′ and 3′ UTR have been replaced by MS2 regions.

FIG. 20 is a graphical depiction exhibiting the effect that altered payloads have on integration. HEK293FT cells were transfected with 3 separate plasmids: the first containing an R2 protein encoding region, the second containing a luciferase reporter region with 26/22 bp R2 landing sites, and the third (payload) plasmid containing the second artificial exon necessary for luciferase signal after insertion and splicing of the reporter plasmid. Additionally to a small intronic sequence upstream of the second of two artificial luciferase exons (minimal cargo necessary for luciferase signal), the payload plasmid has 5′ and 3′ UTR sequences as well as 5′ and 3′ appended at the 3′ end with a number of different nuclear retention elements, as named on the x-axis. Figure discloses “atcTgtcaGtaAGCCCcatgGaAA” as SEQ ID NO: 33547.

FIG. 21 is a graphical depiction exhibiting the effect that altered payloads have on integration. HEK293FT cells were transfected with 3 separate plasmids: the first containing an R2 protein encoding region, the second containing a luciferase reporter region with 26/22 bp R2 landing sites and the third (payload) plasmid containing the second artificial exon necessary for luciferase signal after insertion and splicing of the reporter plasmid. Additionally to a small intronic sequence upstream of the second of two artificial luciferase exons (minimal cargo necessary for luciferase signal), the payload plasmid has 5′ and 3′ UTR sequences and modifications thereof as named on the x-axis, as well as 5′ and 3′ homologies.

FIG. 22A is a graphical depiction of luminescence readout of HEK293FT cells transfected with three separate plasmids, indicating cleavage by Cas9. HEK293FT cells were transfected with 3 separate plasmids. The first plasmid either contained modified R2/Cas9 fusion protein, linked together by an XTEN sequence. The second plasmid contained a luciferase reporter region for Cas9 cleavage. The third plasmid a single guide RNA. Columns 1-3 display the results when the transfected R2 protein is an R2 protein in which the −1 domain, which is an RNA interaction domain, has been deleted. Gray bars indicate an R2 protein with a nuclear localization signal, while orange bars indicate an R2 protein without a nuclear localization signal. The x-axis lists the individual R2/Cas9 fusion proteins tested, as well as PDY0044 and a positive control. FIG. 22B is a visual representation of the modified fusion proteins used in FIG. 20A. Vertical lines where in the R2 protein the Cas9 portion is linked to the R2 portion by the XTEN linker.

FIG. 23 is a visual representation exhibiting the integration of a 20 bp sequence to trigger the expression of GFP using a modified Cas9/R2 protein. FIGS. 23A-N represent modified fusion proteins of Cas9 fused at the N-terminus to R2 at varying locations. The fusion proteins of FIGS. 23A-N exhibit the ability to insert a missing 20 bp region into an eGFP precursor (FIG. 23Q), leading to GFP expression.

FIG. 230 is a negative control and FIG. 23P is a positive control.

FIG. 24A is a schematic of computational pipeline used to discover and classify site-specific non-LTR retrotransposon systems. Figure discloses SEQ ID NOS 33548-33553 and 33553-33554, respectively, in order of appearance. FIG. 24B-C is a visual representation of a Phylogenetic tree of single-ORF non-LTR retrotransposons. Associations with putative target sites, including tandem repeats and conserved RNA families are shown. Full length ORF size is shown in the outermost ring with associated domains shown in inner rings. Labels of specific retrotransposons orthologs used in this study as well as previously described orthologs are listed above the outer ring with associated symbols labeled on the tree. Tandem repeat GC content percentage is shown as a color scale. Protein domains are colored according to different CDD/Pfam domains analyzed. Putative Myb and zinc finger domains from Prosite and Pfam (ZF) are colored according to the different configurations detected. The 9 families of RLE-containing non-LTR retrotransposons are shaded in different colors and labeled. SL1, corresponds to SL1 spliced-leader RNA. LSU, corresponds to large subunit rRNA (28S). SSU, corresponds to small subunit rRNA (18S). ZF motif labels correspond to different pfam IDs. CDD labels correspond to different CDD IDs.

FIG. 25 is a visual representation of the Size distribution of the ORFs from the first methionine for each of the 9 families of RLE containing non-LTR retrotransposons.

FIG. 26A is a schematic of chimeric non-LTR (nLTR) retrotransposon systems with flanking homologies targeting different insertion sites. E) Gaussia luciferase (Gluc) production via payload insertion of a synthetic exon 2 by selected non-LTR retrotransposons into a 28S plasmid reporter, normalized to a Cypridina luciferase (Cluc) control. FIG. 26B is a schematic of typical non-LTR retrotransposon insertion sites with target sites consistent on both sides of the retrotransposon.

FIG. 27A is a visual analysis of results from a multiple sequence alignment of different non-LTR retrotransposons using MUSCLE, with Pfam domain schematic above as determined by HHpred. FIG. 27B is a visual analysis of sequence identity similarity of chosen non-LTR retrotransposon family members using the MUSCLE protein alignment from E.

FIG. 28 is a visual analysis of the 5′ end of the R10Mbr locus with the microsatellite repeat region and alignment to the human 28S rDNA region highlighted. Figure discloses SEQ ID NOS 33555-33557, respectively, in order of appearance.

FIG. 29A is an analysis of Gaussia luciferase (Glue) production via payload insertion of a synthetic exon 2 by selected non-LTR retrotransposons into a 28S plasmid reporter, normalized to a Cypridina luciferase (Cluc) control. FIG. 29B is a schematic of payload homology and target sites used to evaluate R10Mbr insertion. Figure discloses SEQ ID NOS 33558-33562, respectively, in order of appearance. FIG. 29C is a visual analysis of the results of an experiment analyzing Gluc payload insertion by R10Mbr into a panel of luciferase reporters, as quantified by luciferase production, with R2Tg targeting the R2 28S sequence as control. Reporters with either similarity to the R2 28S region, or with similarity to the 28S homology region in the R10Mbr locus are used for evaluation of alternative insertion sites.

FIG. 30A is an analysis of EGFP payload insertion by wild type and domain inactivated mutants of R2Tg at the endogenous human 28S locus, analyzed at 5′ and 3′ junctions via gel electrophoresis. Mutants tested were D1274A (RLE inactivation), D877A/D878A/D884A (RT domain inactivation), and ZF2 domain inactivation (replacement of residues 262-275 with NCp7 ZF FNCGKEGHTARN (SEQ ID NO: 33379) (Rocquigny, et al., (1997) J. Biol. Chem. 272, 30753-30759) Red triangles denote faint insertion bands. Schematic above shows insert with the payload denoted in blue, UTRs denoted in black, 28S homology arms denoted red, and 28S locus denoted grey. Black primers are used to readout the left junction and gold primers are used to readout the right junction. FIG. 30B is an analysis of EGFP payload insertion by wild type and domain inactivated mutants of R2Tg into the endogenous 28S locus, quantified by next-generation sequencing. FIG. 30C is an analysis of Gluc production by wild type and domain inactivated mutants of R2Tg into a 28S plasmid reporter, normalized to a Cluc control.

FIG. 31A is graphical analysis of Gaussia luciferase exon 2 (Gluc) payload insertion by wild type and domain inactivated mutants of R2Tg into a 28S plasmid reporter, with editing outcomes profiled by next generation sequencing at the upstream (left) junction. Mutants tested are WT R2Tg and R2TgD1274A, R2TgD877A, _{D878A, D884A}, and R2TgZF2mut, and outcomes are classified as perfect insertions, insertions with indels, or WT locus indels. FIG. 31B is a graphical analysis of Gluc payload insertion by wild type and domain inactivated mutants of R2Tg into a 28S plasmid reporter, with editing outcomes profiled by next generation sequencing at the downstream (right) junction. Mutants tested are WT R2Tg and R2TgD1274A, R2TgD877A, _{D878A, D884A}, and R2TgZF2mut, and outcomes are classified as perfect insertions, insertions with indels, or WT locus indels. FIG. 31C are representative edits at the 5′-insertion junction, showing examples of indels in the outcome insertion products. Figure discloses SEQ ID NOS 33563-33565, respectively, in order of appearance.

FIG. 32A is a schematic of example N- and C-terminal R2Tg truncations for evaluating domain functionality. Not all truncations shown. FIG. 32B is a graphical analysis of Gluc payload insertion by wild type and N- or C-terminal truncations of R2Tg into a 28S plasmid reporter, quantified by next-generation sequencing.

FIG. 33A is a schematic of Cas9H840A-R2Tg insertion at the 28S target, allowing for rescue of R2TgZF2mut activity. FIG. 33B is a graphical analysis of guide-programmed Gluc payload insertion by SpCas9H840A-R2TgZF2mut into a 28S plasmid reporter, in combination with paired guides or single guides, quantified by next generation sequencing. Perfect insertions, insertions with indels, and pure indel outcomes of Cas9H840A-R2TgZF2mut fusion are compared to SpCas9H840A. FIG. 33C is a graphical analysis of Gluc payload insertion by WT R2Tg into a 28S plasmid reporter, with editing outcomes profiled by next generation sequencing. Outcomes are classified as perfect insertions, insertions with indels, or WT locus indels.

FIG. 34A is a graphical analysis of a Gluc payload insertion by dead SpCas9D10A, H840A-R2Tg and mutants with targeting and non-targeting guides into a 28S plasmid reporter, as quantified by luciferase production. FIG. 34B is a graphical analysis of a Gluc payload insertion by domain inactivated versions of SpCas9H840A-R2Tg into a 28S plasmid reporter and quantified by luciferase production and normalized to the corresponding SpCas9H840A guide condition. SpCas9H840A-R2Tg is combined with either dual, single, or nontargeting sgRNA combinations. Variants tested are R2TgD1274A and R2TgZF2mut. FIG. 34C is a graphical analysis of a Gluc payload insertion by wild type and domain inactivated mutants of SpCas9H840A-R2Tg fusion into a 28S plasmid reporter, quantified by luciferase production and normalized to SpCas9H840A.

FIG. 35A is a schematic for homology length titration of R2Tg payloads, with varying 5′ and 3′ homology lengths (red). The Gluc cargo is shown in blue. Hairpins denote the 5′ and 3′ UTRs. FIG. 35B is a graphical analysis of a Gluc payload insertion by R2Tg into a 28S plasmid reporter with payloads of different 5′ or 3′ homology lengths, profiled by next generation sequencing. Editing outcomes are quantified as perfect insertions, insertions with indels, and pure indels. FIG. 35C is a schematic for R2Tg insertion outcomes at the 28S target site, either with or without scars, with junction amplification primers for Sanger sequencing and gel readouts shown. Black and gold primers are used for 5′ and 3′ junction analyses, respectively. Schematic shows payload denoted in blue, UTRs denoted in black, 28S homology arms denoted red, and 28S locus denoted grey.

FIG. 36A is a schematic of R2Tg scarless payload designs, with permuted and deleted UTR domains. FIG. 36B Sanger sequencing of 5′ and 3′ insertion junctions at the 28S target for additional selected payload designs after R2Tg integration. Payload numbers correspond to those in FIG. 36A. Figure discloses SEQ ID NOS 33566-33567, respectively, in order of appearance. FIG. 36C is a visual depiction of Sanger sequencing of 5′ and 3′ insertion junctions at the 28S target for selected payload designs after R2Tg integration. Payload numbers correspond to those in 36A. Figure discloses SEQ ID NOS 33566, 33568-33569, 33568-33569, 33567, 33569, and 33567, respectively, in order of appearance.

FIG. 37A is a visual representation of edits at the 5′ insertion junction, showing examples of indels in the outcome insertion products. Figure discloses SEQ ID NOS 33563-33565, respectively, in order of appearance. FIG. 37B is a visual depiction of indels at the 5′ junction for R2Tg insertion at the 28S target for selected payloads. Non-templated Cs from reverse transcription in the bottom strand (G in the top strand) are highlighted with red boxes. Figure discloses SEQ ID NOS 33570-33571, 33564, 33572, 33571, 33564, 33582, and 33571, respectively, in order of appearance. FIG. 37C is a visual depiction of a size analysis by gel of 5′ and 3′ insertion junctions at the 28S target reporter for selected payload designs after R2Tg integration. Payload numbers correspond to those in FIG. 36A.

FIG. 38A is a graphical depiction of integration efficiency of R2Tg at the 28S target reporter with different payload designs. Integration is profiled by next-generation sequencing as perfect insertions, insertions with indels, or WT locus indels. Payload numbers correspond to those in FIG. 36A. FIG. 38B is a visual depiction of example indels at the WT 28S locus target for selected payloads. Non-templated Cs from reverse transcription in the bottom strand (Gin the top strand) are highlighted with red boxes. Figure discloses SEQ ID NOS 33563, 33565, 33564, 33571, 33564, 33573, 33571, 33564-33565, and 33573, respectively, in order of appearance. FIG. 38C is a schematic representation of additional payload variant with internal homology arms against the 28S target. FIG. 38D is a graphical representation of the Gaussia luciferase exon 2 (Gluc) payload insertion by wild type R2Tg into a 28S plasmid reporter with payload variants shown in part B, with editing outcomes profiled by next generation sequencing at the upstream (left) junction. Outcomes are classified as perfect insertions, insertions with indels, or WT locus indels.

FIG. 39A is a schematic for reprogramming of a R2Tg payload for insertion at the AAVS1 site with scarless insertion. FIG. 39 B is a graphical depiction of a payload insertion by SpCas9H840A-R2Tg into the endogenous NOLC1 and AAVS1 loci, mediated by either single, dual guides, or non-targeting guides and quantified by next generation sequencing. FIG. 39C is a schematic of AAVS1 targeting payload variations used in FIG. 39D. Payload is shown in blue, homology arms are shown in gold, 5′ 28S homology is shown in red, and UTRs are shown as hairpins. FIG. 39D is a graphical depiction of a Gluc payload insertion, with variations on UTR, 28S homology, and AAVS1 homology (100 nt), by SpCas9H840A-R2Tg at endogenous AAVS1 locus, using a single bottom strand nicking guide. Integration is profiled by next-generation sequencing as perfect insertions, insertions with indels, or indels.

FIG. 40A is a schematic of SpCas9H840A fused to N- and C-terminal truncations of R2Tg at different amino acid positions. Not all tested constructs are shown. FIG. 40B is a graphical depiction of a Gluc payload insertion by different SpCas9H840A-R2Tg fusions, according to the schematic in A, into the endogenous AAVS1 locus quantified by next generation sequencing. FIG. 40C is a graphical depiction of the payload insertion by SpCas9H840A-R2Tg fusion, SpCas9D10A,_{H840A}-R2Tg fusion, and SpCas9H840A and R2Tg in trans. Payloads are inserted at either AAVS1 or NOLC1 loci, with insertion at AAVS1 quantified by next generation sequencing and insertions at NOLC1 quantified by ddPCR.

FIG. 41A is a graphical depiction of a Gluc payload insertion by SpCas9H840A-R2Tg at the endogenous AAVS1 target site with a panel of dual and single guides, compared with SpCas9H840A. Payloads have 100 nt of homology to the target site. Editing outcomes are quantified as perfect insertions, insertions with indels, and indels at the unmodified target site. The optimized payload design is used with a 5′ 28S homology arm, truncated 5′ R2Tg UTR, and internal AAVS1 homology arms. FIG. 41B is a graphical depiction of the integration of Gluc payload at the endogenous AAVS1 locus by the SpCas9H840A-R2Tg fusion with a payload containing 50 nt homology arms.

FIG. 42A is a graphical depiction of a Gluc payload insertion into a 28S plasmid reporter by selected non-LTR retrotransposons fused to SpCas9H840A, with either targeting or non-targeting guides, quantified by Gluc production normalized to a control Cluc. Data is shown as ratio of targeting signal to non-targeting signal. FIG. 42B is a schematic of AAVS1 insertion with optimized payloads containing the cognate 5′ UTR corresponding to each non-LTR retrotransposon ortholog being evaluated. FIG. 42C is a graphical depiction of a Gluc payload insertion into the endogenous AAVS1 locus by selected non-LTR retrotransposons fused to SpCas9H840A, with either targeting or non-targeting guides, quantified by next generation sequencing. FIG. 42D Gluc payload insertion into the endogenous AAVS1 locus by selected non-LTR retrotransposons fused to SpCas9H840A, with either targeting or non-targeting guides, profiled by next generation sequencing. Editing outcomes are quantified as perfect insertions, insertions with indels, and indels at the unmodified WT target site.

FIG. 43A is a graphical depiction of EGFP payload insertion (50 nt homology arms) by STITCHR with SpCas9H840A-R2Toc into the endogenous NOLC1 locus, with combinations of single and dual guides, compared to SpCas9H840A and quantified by digital droplet PCR (ddPCR). Editing outcomes are quantified as total insertions, integrations with indels, and WT locus indels. FIG. 43B is a graphical depiction of a Gluc payload insertion by STITCHR with SpCas9H840A-R2Toc into the endogenous SERPINA1 locus (left homology 100 nt and right homology 50 nt), with combinations of single and dual guides, compared to SpCas9H840A and profiled by next generation sequencing. Editing outcomes are quantified as perfect insertions, insertions with indels, and WT locus indels. FIG. 43C is a graphical depiction of an EGFP payload insertion by STITCHR with SpCas9H840A-R2Toc into the endogenous NOLC1 locus, with combinations of single and dual guides, compared to a non-targeting guide control and quantified by digital droplet PCR (ddPCR). FIG. 43D is a graphical depiction of an EGFP payload insertion by STITCHR with SpCas9H840A-R2Toc into the endogenous NOLC1 locus, with combinations of single and dual guides, compared to a non-targeting guide control and profiled by next generation sequencing. Editing outcomes are quantified as perfect insertions, insertions with indels, and WT locus indels.

FIG. 44A is a graphical depiction of an EGFP payload insertion by STITCHR with SpCas9H840A-R2Toc into the endogenous NOLC1 locus, with a panel of payloads with 50 nt homology arms targeting NOLC1 or AAVS1 targets, or without homology. Payloads are evaluated with single, dual, or non-targeting guides and are compared to SpCas9H840A. Editing is quantified by ddPCR. N denotes the NOLC1 target. A denotes the AAVS1 target. FIG. 44B is a graphical depiction of an EGFP payload insertion by STITCHR with SpCas9H840A-R2Toc into the endogenous NOLC1 locus, with a panel of payloads with varying homology arm lengths. Payloads are evaluated with dual or non-targeting guides and are compared to SpCas9H840A. Editing is quantified by ddPCR. FIG. 44C is a graphical evaluation of gene integration at the AAVS1 locus with SpCas9H840A-R2Toc and SpCas9H840A using payloads of varying sized homology arms (100 nt, 75 nt, 50 nt, and 30 nt). Integration is evaluated with dual guides, single guides, and non-targeting guides. FIG. 44D is a graphical evaluation of gene integration at the SERPINA1 locus with SpCas9H840A-R2Toc and SpCas9H840A using payloads of varying sized homology arms (100 nt, 75 nt, 50 nt, and 30 nt). Integration is evaluated with dual guides, single guides, and non-targeting guides.

FIG. 45A is a schematic of STITCHR using SpCas9H840A-R2Toc to insert EGFP as a scarless in-frame fusion at the N-terminus of the human NOLC1 gene. The EGFP template is transcribed in a reverse complement manner to minimize background expression in the absence of insertion with 50 nt homology arms. FIG. 45B is an immunohistochemical analysis of STITCHR-mediated EGFP tagging of NOLC1, visualized by confocal microscopy, and compared to immunofluorescence staining of NOLC1. White scale bar denotes 10 μm. FIG. 45C is a graphical depiction of therapeutically relevant payload insertion by STITCHR with SpCas9H840A-R2Toc into the endogenous AAVS1 locus, with sizes and identities of payload panel members shown and 100 nt homology arms. Integration is quantified by next generation sequencing and compared to SpCas9H840A. FIG. 45D is a graphical depiction of therapeutically relevant payload insertion by STITCHR with SpCas9H840A-R2Toc into the endogenous AAVS1 locus, compared to SpCas9H840A. Integration is profiled by next-generation sequencing as perfect insertions, insertions with indels, or WT locus indels.

FIG. 46A is a graphical depiction of EGFP payload insertion (50 nt homology arms) by STITCHR with SpCas9H840A-R2Toc into the endogenous NOLC1 locus in cells treated with varying concentrations of aphidicolin. Integration is quantified by ddPCR and compared to SpCas9H840A. FIG. 46B is a graphical depiction of SpCas9-mediated HDR editing of the EMX1 gene in cells treated with varying concentrations of aphidicolin. Genome editing is quantified by next generation sequencing.

FIG. 47A is a graphical depiction of multiplexed gene integration by STITCHR with SpCas9H840A-R2Toc at NOLC1 and AAVS1 sites. EGFP payload insertion at NOLC1 is quantified by ddPCR, and Gluc insertion at AAVS1 is quantified by next generation sequencing. Targeting conditions are compared to non-targeting guide controls. FIG. 47B is a graphical depiction of multiplexed gene integration by STITCHR with SpCas9H840A-R2Toc at NOLC1 and AAVS1 sites, profiled by next generation sequencing. Total insertion for NOLC1 is quantified by ddPCR. Editing outcomes are quantified as perfect insertions, insertions with indels, and WT locus indels. N denotes NOLC1, whereas A denotes AAVS1.

FIG. 48 is a schematic representation of STITCHR, enabling programmable and modular scarless gene insertion with site-specific non-LTR (nLTR) retrotransposons.

FIG. 49 is a graphical representation of the results of an experiment in which an EGFP payload was inserted (50 nt homology arms) by STITCHR with SpCas9H840A-R2Toc into the endogenous NOLC1 locus, with a single fixed guide, compared to SpCas9H840A and quantified by digital droplet PCR (ddPCR). Homology arms on the templates are separated by 0, 50, 100, or 150 bp on the genome causing a deletion to occur followed by simultaneous insertion of the STITCHR EGFP payload. The payload arms are also shifted to match the locations of the single nicking guide and the desired end of the deletion to enable the deletion and subsequent insertion.

FIG. 50A is a graphical representation of payload insertion (50 nt homology arms) by STITCHR with SpCas9^{H840A}-R2Toc into the endogenous NOLC1 locus, with dual guides N4 and N8, compared to SpCas9^{H840A} and quantified by next generation sequencing. The introduced edit is either a mismatch to the genome to demonstrate single base corrections or are small insertions as noted in the x-axis of the plot. FIG. 50B is a graphical representation of payload insertion (50 nt homology arms) by STITCHR with SpCas9H840A-R2Toc into the endogenous NOLC1 locus, with dual guides N4 and N8, compared to SpCas9H840A and quantified by next generation sequencing. The introduced edit is either a mismatch to the genome to demonstrate single base corrections or are small insertions as noted in the x-axis of the plot. Cargo is driven by either the U6 promoter or the CAG promoter, showing that the CAG promoter expression of the cargo results in slightly higher editing.

FIG. 51 is a graphical representation of the results of an experiment in which EGFP payload was inserted (50 nt homology arms) by STITCHR with SpCas9H840A-R2Toc into the endogenous NOLC1 locus, with dual guides N4 and N8, compared to SpCas9H840A and quantified by digital droplet PCR (ddPCR). STITCHR insertion is also compared to SpCas9H840A and R2Toc being expressed separately (in trans).

FIG. 52 is a heatmap chart representation of nLTR families with diverging target preferences, with counts of co-occurring divergent Rfam annotation target pairs.

FIG. 53 are loci of nLTR system families with divergent target preferences as determined via Rfam analysis. Families are clustered by ORF identity.

FIG. 54A is a schematic representation of the insertion by non-LTR retrotransposons at the natural 28S target site, depicting initial nicking and strand invasion, target-primed reverse transcription, first strand synthesis, nicking-initiated second strand synthesis, and insertion of a payload sequence into the genome. 28S homology, UTR sequences, and payload sequence are indicated. FIG. 54B is a schematic representation of Gaussia luciferase (Gluc) production via payload insertion of a synthetic Gluc exon 2 by 12 selected non-LTR retrotransposons into a 28S plasmid reporter containing a synthetic Gluc exon 1, normalized to a constitutive Cypridina luciferase (Cluc) control. FIG. 54C is a schematic representation of Gluc exon 2 payload insertion by R2Tg into a 28S plasmid reporter with payloads of different 5′ or 3′ UTR deletions and homology site permutations, profiled by next generation sequencing. Schematic shows the payload design used with UTRs, 5′ 28S homology arms, 3′ 28S homology arms, and the Gluc exon 2 insert.

FIG. 55A are gel electrophoresis images of the analysis of 5′ and 3′ insertion junctions at the 28S target reporter using payload designs with permuted UTR and homology positions after R2Tg integration. Payload numbers correspond to those in FIG. 54C. FIG. 55B is a schematic representation of the Gluc exon 2 payload insertion by WT R2Tg, R2Tg^{D1274A,} or the RT domain deletion R2Tg^{Δ(874-884)} into a 28S plasmid reporter with payloads containing 28S or AAVS1 targeting homology arms, profiled by next generation sequencing. FIG. 55C is a graphical representation of the EGFP payload insertion at the NOLC1 target using R2Tg, R2Tg^{D1274A}, or R2Tg^{RTmut} and a payload containing the 5′ UTR and 50 nt NOLC1 homology arms, quantified by next-generation sequencing.

FIG. 56A is a schematic representation of the reprogramming of a R2Tg payload for insertion at a novel site with scarless insertion using SpCas9^{H840A}. FIG. 56B is a graphical representation of the payload insertion by SpCas9^{H840A}-R2Tg or SpCas9^{H840A}-R2Tg^{D1274A} into the endogenous NOLC1 locus, mediated by dual guides or non-targeting guides and quantified by ddPCR.

FIG. 57 is a schematic representation of the EGFP payload insertion, with variations on 5′ and 3′ UTR sequence by SpCas9^{H840A}-R2Tg at the endogenous NOLC1 locus, using dual guides. Integration is quantified by ddPCR. Schematic of payload variations used with the payload, homology arms, 5′ and 3′ UTRs are illustrated.

FIG. 58A is a graphical representation of the EGFP payload insertion by SpCas9^{H840A}-R2Tg (WT), SpCas9^{H840A}-R2Tg^{F875A/A876L/D877A/D878A/L879A/V880A/L881A} (RTmut), and SpCas9^{H840A}-R2Tg^{Δ(874-884)} (Δ(874-884)), and SpCas9^{H840A} at the endogenous NOLC1 target site with dual guides. FIG. 58B is a schematic representation of AAVS1 insertion with optimized payloads containing the cognate 5′ UTR corresponding to each non-LTR retrotransposon ortholog being evaluated. Gluc payload insertion into the endogenous AAVS1 locus by selected non-LTR retrotransposons fused to SpCas9^{H840A}, with either targeting or non-targeting (NT) guides, is quantified by next generation sequencing. The heatmaps correspond to Gluc integration efficiency (top) and the associated indels generated at the AAVS1 locus (bottom).

FIG. 59A is a schematic representation of the EGFP payload insertion (50 nt homology arms) by STITCHR with SpCas9^{H840A}-R2Toc into the endogenous AAVS1, LMNB1, EMX1, and NOLC1 loci, with combinations of single and dual guides, compared to SpCas9^{H840A}-R2TocRTmut and wild-type SpCas9. The left heatmap shows integration rate of the EGFP payload, whereas the right heatmap corresponds to indels detected at the corresponding loci. FIG. 59B is a schematic representation of different STITCHR edits evaluated ranging from single-base variants, small insertions, and large insertions. FIG. 59C is a graphical representation of the evaluation of different sized edits using STITCHR at the NOLC1 locus using either SpCas9^{H840A}-R2Toc or SpCas9^{H840A}.

FIG. 60A is a schematic representation of STITCHR-replace methodology involving replacement of a region of the genome while inserting the STITCHR payload. FIG. 60B is a graphical representation of the evaluation of STITCHR-replace at the NOLC1 locus using a single guide and homology arms spaced 50-150 bp apart on the genome.

FIG. 61 is a schematic representation of the natural reprogramming of RLE-containing non-LTR retrotransposons, incorporating flexible internal priming and UTR deletions that might occur during the process.

FIG. 62 is a graphical representation of the distribution of distances from candidate retrotransposons to detected Rfam annotation or tandem repeat targets for each of the 9 families of RLE containing non-LTR retrotransposons.

FIG. 63 is the phylogenetic tree representation of 9 families of RLE-containing nLTR systems showing majority of detected Rfam targets in the vicinity of the nLTR ORF.

FIG. 64A-E are the DNA sequence alignments of nLTR families with divergent target preferences in the noncoding areas surrounding the nLTR ORFs. Identified Rfam annotations in the surrounding locus are highlighted.

FIG. 65A is the graphical representation of the Gluc payload insertion by R2Tg reverse transcriptase domain deletions, RLE inactivation mutants (R1274A) and reverse transcriptase mutations (R2Tg^{F875A/A876L/D877A/D878A/L879A/V880A/L881A}, RTmut), at the 28S locus luciferase reporter, as quantified by luciferase. FIG. 65B is the graphical representation of the Gluc payload insertion by R2Tg reverse transcriptase domain mutations, including R2Tg^{F875A/A876L/D877A/D878A/L879A/V880A/L881A} (RTmut) and RLE inactivation mutants (R1274A), at the 28S locus luciferase reporter, as quantified by luciferase.

FIG. 66A is a schematic representation of the secondary structure analysis of the 5′ UTR of R2Tg, including the full length, 15 nt truncated variant, and the 15 nt truncated variant with the 50 nt 28S homology sequence upstream. Figure discloses SEQ ID NOS 33574-33576, respectively, in order of appearance. FIG. 66B is a graphical representation of the validation of the 3-primer NGS assay for analysis of AAVS1 integration via the left insertion junction. Standards consist of edited and WT amplicons that are mixed in the listed ratios (xaxis) and the measured editing is determined by the 3-primer NGS assay (y-axis). FIG. 66C is the schematic and graphical representation of the Gluc integration at the endogenous AAVS1 locus via the SpCas9^{H840A}-R2Tg fusion using payloads with the full length or 15-nt truncated 5′ UTR, an upstream 28S 50 nt sequence, and internal AAVS1 homology arms. Integration is quantified by next-generation sequencing.

FIG. 67A is a schematic representation of SpCas9^{H840A} fused to N- and C-terminal truncations of R2Tg at different amino acid positions. Not all tested constructs are shown. FIG. 67B is a graphical representation of the Gluc payload insertion by different SpCas9^{H840A}-R2Tg fusions, according to the schematic in FIG. 67A, into the endogenous AAVS1 locus quantified by next generation sequencing. FIG. 67C is a graphical representation of the Gluc integration at the endogenous AAVS1 target by SpCas9^{H840A}-R2Tg, SpCas9^{H840A}-R2Tg^{F875A/A876L/D877A/D878A/L879A/V880A/L881A}, and SpCas9^{H840A}-R2Tg^{Δ(874-884)}, and SpCas9^{H840A} alone.

FIG. 68 is a schematic representation of the Gluc payload insertion into the endogenous AAVS1 locus by selected non-LTR retrotransposons fused to SpCas9^{H840A}, with either targeting or nontargeting guides, profiled by next generation sequencing. Editing outcomes are quantified as perfect insertions, insertions with indels, and indels at the unmodified WT target site

FIG. 69A is a graphical representation of the Gluc payload insertion by STITCHR with SpCas9^{H840A}-R2Toc into the endogenous AAVS1 locus, with combinations of single and dual guides, compared to a non-targeting guide control and quantified by next generation sequencing. FIG. 69B is a graphical representation of the EGFP payload insertion by STITCHR with SpCas9^{H840A}-R2Toc into the endogenous LMNB1 locus, with combinations of single and dual guides, compared to a non-targeting guide control and SpCas9^{H840A} alone. Editing was quantified by digital droplet PCR (ddPCR). FIG. 69C is a graphical representation of the EGFP payload insertion by STITCHR with SpCas9^{H840A}-R2Toc into the endogenous EMX1 locus, with combinations of single and dual guides, compared to a non-targeting guide control and SpCas9^{H840A} alone. Editing was quantified by digital droplet PCR (ddPCR).

FIG. 70A is a graphical representation of the Gluc payload insertion by SpCas9^{H840A}-R2Toc (WT), SpCas9^{H840A}-R2Toc^{F811A, A812L, D813A, D814A, L815A, V816A, L817A}, SpCas9^{H840A}-R2Toc^{Δ(811-814)}, SpCas9^{H840A}-R2Toc^{Δ(810-820)}, and SpCas9^{H840A} at the endogenous AAVS1 target site. Editing is quantified by next generation sequencing. FIG. 70B is a graphical representation of the EGFP payload insertion by SpCas9^{H840A}-R2Toc (WT), SpCas9^{H840A}-R2Toc^{F811A, A812L, D813A, D814A, L815A, V816A, L817A}, SpCas9^{H840A}-R2Toc^{Δ(875-878)}, SpCas9^{H840A}-R2Toc^{Δ(874-884)}, and SpCas9^{H840A} at the endogenous NOLC1 target site. Editing is quantified by ddPCR. FIG. 70C is a graphical representation of the GFP payload insertion by SpCas9^{H840A}-R2Toc (WT), SpCas9^{H840A}-R2Toc^{D1210A}, and SpCas9^{H840A} at the endogenous NOLC1 target site. Editing is quantified by ddPCR.

FIG. 71 is a graphical representation of the GFP payload insertion by STITCHR with SpCas9^{H840A}-R2Toc into the endogenous NOLC1 locus in HepG2 cells, compared to SpCas9^{H840A}. Editing is quantified by ddPCR.

FIG. 72 is a graphical representation of the installation of small edits and insertions using STITCHR at the NOLC1 locus, using a U6 promoter for payload expression.

FIG. 73 are sequencing reads of the EGFP insertion site at NOLC1 for STITCHR replace, showing the desired 50-150 bp deletions. Figure discloses SEQ ID NOS 33577-33578, 33577, 33577, 33577, 33579, 33579, 33579, 33579-33580, 33580, 33580, 33580-33581, 33581, 33581, and 33581, respectively, in order of appearance.

FIG. 74A is a graphical representation of the EGFP payload insertion (50 nt homology arms) by STITCHR with SpCas9^{H840A}-R2Toc into the endogenous AAVS1 locus in cells treated with cell cycling inhibitor Mirin or double thymidine. Integration is quantified by next-generation sequencing and compared to SpCas9^{H840A}. FIG. 74B is a graphical representation of the SpCas9-mediated HDR editing of the EMX1 gene in cells treated with cell cycling inhibitor Mirin or double thymidine. Genome editing is quantified by next generation sequencing.

FIG. 75 is a graphical representation of 10 orthologs sampled from various nLTR families (1, 4, 5, 6, 7, 9) compared to R2Toc for programmed insertion at the AAVS1 locus. Orthologs were synthesized with mammalian codon optimization, and putative 5′ and 3′ UTR regions were cloned surrounding a luciferase payload. Protein and payload constructs were transfected along with a SpCas9 plasmid and guide plasmid into HEK293FT cells, and 3 days later cells were harvested and efficiency of insertion were quantified by next generation sequencing.

FIG. 76A-C are tables showing plasmid vectors for genome editing.

### DETAILED DESCRIPTION

The present disclosure is directed to site specific non-Long Terminal Repeat (LTR) retrotransposons and systems incorporating these non-LTR retrotransposons for inserting large nucleic acids at targeted locations within a genome. The present disclosure is also directed to site-specific non-LTR retrotransposons and related systems for performing small nucleotide changes in a genome. In some embodiments, a small nucleotide change comprises a point mutation. In some embodiments, a small nucleotide change comprises a small nucleotide insertion.

The present disclosure is also directed to modified R2 fusion proteins for inserting large nucleic acids at targeted locations within a genome. The present disclosure is also directed to Cas9 fusion proteins for inserting large nucleic acids at targeted locations within a genome, which includes Cas9-R2 fusion proteins. In some embodiments, the genome is a human genome.

The present disclosure is also directed to the insertion of exogenous R2 landing sites within a genome, such that a R2 protein, modified R2 protein, or R2 fusion protein that may target a non-28S locus for insertion of a large genetic element. In some embodiments, the R2 fusion protein is an R2-Cas9 fusion protein. In some embodiments, the R2 fusion protein is a Cas12-R2 fusion protein. In some embodiments, the R2 fusion protein is a TALEN-R2 fusion protein.

### Definitions

Unless stated otherwise, terms and techniques used within this application have the meaning generally known to one of skill in the art.

The term “about” as used herein is understood to modify the specified value. Unless explicitly stated otherwise, the term about is understood to modify the specified values +/−10%. As used herein, the term about applied to a range modifies both endpoints of the range. By way of example, a range of “about 5 to 10” is understood to mean “about 5 to about 10.”

Unless explicitly stated otherwise, the term “payload” as used herein means at least a nucleic acid that may be integrated into a host genome. Thus, “payload RNA” will be understood to comprise an RNA molecule comprising at least an insertion region, wherein the insertion region can be integrated into a host genome.

As used herein, “cell-specific,” or “cell-type specific,” would be understood by one of skill in the art to mean occurring or being expressed at a higher frequency or existing at an increased level in one cell type in contrast to other cell types.

As used herein, the terms “target site” and “landing site” are used interchangeably unless specified otherwise.

Unless explicitly stated otherwise, the term “nucleic acid” is understood to refer to both ribonucleic acid (RNA) and deoxyribonucleic acid (DNA) molecules. This may include chemically synthesized nucleic acid molecules, single stranded or double stranded nucleic acid molecules, linearized nucleic acid molecules, circularized nucleic acid molecules, chemically modified nucleic acid molecules, and nucleic acids with biochemical modifications.

### RLE Domain Containing Non-LTR Retrotransposon Families

In addition to canonical single-ORF RLE domain containing non-LTR retrotransposons, such as R2, R4, R5, R8, R9, Dong, and Cre families, retrotransposons for use in or as part of the genome editing system described herein may also be characterized as part of a larger phylogenetic family. The retrotransposons in these larger phylogenetic families contemplated for use in or as a part of the genome editing systems described herein include the 8,248 RLE-domain containing retrotransposon uncovered as part of the computational analysis described in Example 7. These 8,248 retrotransposon-like orthologs are divided into 9 families, termed RLED1-RLED9. In some embodiments, the non-LTR retrotransposon is a member of the RLED1 family. In some embodiments, the non-LTR retrotransposon is a member of the RLED2 family. In some embodiments, the non-LTR retrotransposon is a member of the RLED3 family. In some embodiments, the non-LTR retrotransposon is a member of the RLED4 family. In some embodiments, the non-LTR retrotransposon is a member of the RLED5 family. In some embodiments, the non-LTR retrotransposon is a member of the RLED6 family. In some embodiments, the non-LTR retrotransposon is a member of the RLED7 family. In some embodiments, the non-LTR retrotransposon is a member of the RLED8 family. In some embodiments, the non-LTR retrotransposon is a member of the RLED9 family. In some embodiments, the non-LTR retrotransposon is a member of the R1 family. In some embodiments, the non-LTR retrotransposon is a member of the R2 family. In some embodiments, the non-LTR retrotransposon is a member of the R4 family. In some embodiments, the non-LTR retrotransposon is a member of the R5 family. In some embodiments, the non-LTR retrotransposon is a member of the R6 family. In some embodiments, the non-LTR retrotransposon is a member of the R7 family. In some embodiments, the non-LTR retrotransposon is a member of the R8 family. In some embodiments, the non-LTR retrotransposon is a member of the R9 family. In some embodiments, the non-LTR retrotransposon is a member of the Cre family. In some embodiments, the non-LTR retrotransposon is a member of the NeSL family. In some embodiments, the non-LTR retrotransposon is a member of the HERO family. In some embodiments, the non-LTR retrotransposon is a member of the Utopia family.

### R2 Element Enzymes

Without limiting the instant disclosure to any one particular theory, R2 retrotransposons are thought to work via a mechanism known as target-primed reverse transcription, or “TPRT.” TPRT is a mechanism by which an endonuclease creates a nick in a first DNA strand at a specific location, creating a “primed” 3′ hydroxyl end for reverse transcription. After the initial DNA nick, an mRNA molecule is reverse transcribed by the reverse transcriptase.

In some embodiments, the R2 element enzyme is modified. In some embodiments, the R2 element enzyme is modified by an N-terminal truncation of the R2 element enzyme sequence, a C-terminal truncation of the R2 element enzyme sequence, or both an N-terminal and a C-terminal truncation of the R2 element enzyme sequence.

In some embodiments, the R2 element enzyme is a fusion protein. In some embodiments, the R2 element enzyme comprises a fusion of an R2 protein with a Cas9 protein. In some embodiments, the R2 element enzyme comprises a fusion of an R2 protein with a Cas12 protein. In some embodiments, the R2 element enzyme comprises a fusion of an R2 protein with a Cas9 protein, wherein the Cas9 portion and the R2 protein portion are connected by a linker. In some embodiments, the R2 element enzyme comprises a fusion of an R2 protein with a Cas12 protein, wherein the Cas12 portion and the R2 protein portion are connected by a linker.

### Protein Binding Elements

Protein binding elements of the disclosure can come in a multitude of forms. In one embodiment, a protein binding element may be an endogenous nucleic acid sequence. In one embodiment, a protein binding element may be an exogenous or introduced nucleic acid sequence. In one embodiment, the protein binding element may be a synthesized nucleic acid sequence.

### Guide Elements

In some embodiments the genome editing system comprises a guide RNA. In some embodiments, the genome editing system comprises multiple guide RNAs. In some embodiments, the genome editing system comprises paired guide RNAs.

### Genomic Insertion Sites and Targets

The R2 element naturally targets the 28S rRNA locus. The instant disclosure contemplates the insertion of payloads into either the 28S rRNA locus or into other genomic loci. In some embodiments, the insertion site is a targeted genomic insertion site. In some embodiments, the insertion site is targeted by a targeting domain in a fusion protein. In some embodiments, the insertion site has been exogenously introduced to the genome. In some embodiments, the insertion site has been exogenously introduced by a site-directed genome editing system that is not capable of delivering large genetic insertions. In some embodiments, the targeted genomic site is targeted for a point mutation. In some embodiments, the targeted genomic site is targeted for a small nucleotide insertion.

The instant disclosure also contemplates additional non-LTR site-specific retrotransposons for use in or as part of the genome editing system described herein that do not target the 28S rRNA locus. In some embodiments, the genome is targeted for a large genetic insertion. In some embodiments, the insertion site is a targeted genomic insertion site. In some embodiments, the insertion site is targeted by a targeting domain in a fusion protein. In some embodiments, the insertion site has been exogenously introduced to the genome. In some embodiments, the insertion site has been exogenously introduced by a site-directed genome editing system that is not capable of delivering large genetic insertions. In some embodiments, the targeted genomic site is targeted for a point mutation. In some embodiments, the targeted genomic site is targeted for a small nucleotide insertion.

### Payloads

Payloads of the instant disclosure may encode proteins, such as enzymes. In some embodiments, the payload may act as a regulatory element. Thus, if an embodiment of the disclosure states, by way of example, that “the payload comprises a therapeutic protein,” it is generally understood that the payload comprises a template that, upon insertion, will lead to expression of a therapeutic protein encoded by the template. Exemplary vectors for expression are shown in FIG. 76.

In some embodiments, the insertion region comprises a template for a reporter gene. In some embodiments, the reporter gene encodes a fluorescent protein. In some embodiments, the reporter gene encodes a green fluorescent protein. In some embodiments, the reporter gene encodes eGFP.

In some embodiments, the insertion region comprises a template for a transcription factor gene.

In some embodiments, the insertion region comprises a template for a transgene.

In some embodiments, the insertion region comprises a template for an enzyme gene, or a therapeutic gene. In some embodiments, the therapeutic protein can be used in conjunction with another therapeutic.

In some embodiments, the payload comprises a protein that is capable of converting one cell type to another.

In some embodiments, the payload comprises a protein that is capable of killing a specific cell type. In some embodiments, the payload comprises a protein that is capable of killing a tumor cell. In some embodiments, the payload comprises an immune modulating protein.

In some embodiments, the payload comprises a 5′UTR. In some embodiments, the payload comprises a 3′UTR. In some embodiments, the payload comprises a 5′UTR and a 3′ UTR. In some embodiments, the payload consists of a 5′UTR. In some embodiments, the payload consists of a 3′UTR. In some embodiments, the payload comprises a 5′UTR and a 5′ homology region. In some embodiments, the payload comprises a 3′UTR and a 3′ homology region. In some embodiments, the payload comprises a 5′UTR, a 5′ homology region, a 3′UTR and a 3′ homology region. In some embodiments, the payload comprises a 5′ homology region, a 3′UTR and a 3′ homology region. In some embodiments, the payload comprises a 5′UTR, a 5′ homology region, and a 3′ homology region. In some embodiments, the payload comprises a 5′ homology region and a 3′ homology region. In some embodiments, the 3′ homology region comprises less than 30 base pairs. In some embodiments the 3′ homology region comprises less than 20 base pairs. In some embodiments, the 3′ homology region comprises less than 10 base pairs. In some embodiments, the 3′ homology region comprises less than 5 base pairs.

### Programmable Nucleases, Nickases, and DNA Binding Proteins

The instant disclosure contemplates programmable nucleases or nickases for use in or as a part of the genome editing systems described herein. In some embodiments, the programmable nuclease or nickase is a Cas9 protein. In some embodiments, the programmable nuclease or nickase is a Cas12 protein. In some embodiments the programmable nuclease or nickase is IscB. In some embodiments, the programmable nuclease or nickase is IsrB. In some embodiments, the programmable nuclease or nickase is TnpB. In some embodiments, the programmable nuclease or nickase is a TALEN nuclease. In some embodiments, the programmable nuclease or nickase is fused to the non-LTR site-specific retrotransposon element. In some embodiments, the programmable nuclease or nickase is non-covalently linked to the non-LTR site-specific retrotransposon element. In some embodiment, the programmable nuclease or nickase acts in cis with the non-LTR site-specific retrotransposon element. In some embodiments, the programmable nuclease or nickase acts in trans with the non-LTR site-specific retrotransposon element.

### Therapeutic Gene Insertions

In some embodiments, the payload results in the insertion of a therapeutic gene into a host genome. In some embodiments, the therapeutic gene is intended to treat a neurological disorder or a neurodegenerative disorder. In some embodiments, the therapeutic gene is intended to treat cancer. In some embodiments, the therapeutic gene is intended to treat an autoimmune disorder.

In some embodiments, the payload results in the insertion of a therapeutic gene for treating a genetically inherited disease. In some embodiments, the genetically inherited disease is Meier-Gorlin syndrome. In some embodiments, the genetically inherited disease is Seckel syndrome 4. In some embodiments, the genetically inherited disease is Joubert syndrome 5. In some embodiments, the genetically inherited disease is Leber congenital amaurosis 10. In some embodiments, the genetically inherited disease is Charcot-Marie-Tooth disease, type 2. In some embodiments, the genetically inherited disease is leukoencephalopathy. In some embodiments, the genetically inherited disease is Usher syndrome, type 2C. In some embodiments, the genetically inherited disease is spinocerebellar ataxia 28. In some embodiments, the genetically inherited disease is glycogen storage disease type III. In some embodiments, the genetically inherited disease is primary hyperoxaluria, type I. In some embodiments, the genetically inherited disease is long QT syndrome 2. In some embodiments, the genetically inherited disease is Sjögren-Larsson syndrome. In some embodiments, the genetically inherited disease is hereditary fructosuria. In some embodiments, the genetically inherited disease is neuroblastoma. In some embodiments, the genetically inherited disease is amyotrophic lateral sclerosis type 9. In some embodiments, the genetically inherited disease is Kallmann syndrome 1. In some embodiments, the genetically inherited disease is limb-girdle muscular dystrophy, type 2L. In some embodiments, the genetically inherited disease is familial adenomatous polyposis 1. In some embodiments, the genetically inherited disease is familial type 3 hyperlipoproteinemia. In some embodiments, the genetically inherited disease is Alzheimer's disease, type 1. In some embodiments, the genetically inherited disease is metachromatic leukodystrophy. In some embodiments, the genetically inherited disease is cancer. In some embodiments, the genetically inherited disease is Uveitis. In some embodiments, the genetically inherited disease is SCA1. In some embodiments, the genetically inherited disease is SCA2. In some embodiments, the genetically inherited disease is FUS-Amyotrophic Lateral Sclerosis (ALS). In some embodiments, the genetically inherited disease is MAPT-Frontotemporal Dementia (FTD). In some embodiments, the genetically inherited disease is Myotonic Dystrophy Type 1 (DM1). In some embodiments, the genetically inherited disease is Diabetic Retinopathy (DR/DME). In some embodiments, the genetically inherited disease is Oculopharyngeal Muscular Dystrophy (OPMD). In some embodiments, the genetically inherited disease is SCAB. In some embodiments, the genetically inherited disease is C9ORF72-Amyotrophic Lateral Sclerosis (ALS). In some embodiments, the genetically inherited disease is SOD1-Amyotrophic Lateral Sclerosis (ALS). In some embodiments, the genetically inherited disease is SCA6. In some embodiments, the genetically inherited disease is SCA3 (Machado-Joseph Disease). In some embodiments, the genetically inherited disease is Multiple system Atrophy (MSA). In some embodiments, the genetically inherited disease is Treatment-resistant Hypertension. In some embodiments, the genetically inherited disease is Myotonic Dystrophy Type 2 (DM2). In some embodiments, the genetically inherited disease is Fragile X-associated Tremor Ataxia Syndrome (FXTAS). In some embodiments, the genetically inherited disease is West Syndrome with ARX Mutation. In some embodiments, the genetically inherited disease is Age-related Macular Degeneration (AMD)/Geographic Atrophy (GA). In some embodiments, the genetically inherited disease is C9ORF72-Frontotemporal Dementia (FTD). In some embodiments, the genetically inherited disease is Facioscapulohumeral Muscular Dystrophy (FSHD). In some embodiments, the genetically inherited disease is Fragile X Syndrome (FXS). In some embodiments, the genetically inherited disease is Huntington's Disease. In some embodiments, the genetically inherited disease is Glaucoma. In some embodiments, the genetically inherited disease is Acromegaly. In some embodiments, the genetically inherited disease is Achromatopsia (total color blindness). In some embodiments, the genetically inherited disease is Ullrich congenital muscular dystrophy. In some embodiments, the genetically inherited disease is Hereditary myopathy with lactic acidosis. In some embodiments, the genetically inherited disease is X-linked spondyloepiphyseal dysplasia tarda. In some embodiments, the genetically inherited disease is Neuropathic pain (Target: CPEB). In some embodiments, the genetically inherited disease is Persistent Inflammation and injury pain (Target: PABP). In some embodiments, the genetically inherited disease is Neuropathic pain (Target: miR-30c-5p). In some embodiments, the genetically inherited disease is Neuropathic pain (Target: miR-195). In some embodiments, the genetically inherited disease is Friedreich's Ataxia. In some embodiments, the genetically inherited disease is Uncontrolled gout. In some embodiments, the genetically inherited disease is Inflammatory pain (Target: Nav1.7 and Nav1.8). In some embodiments, the genetically inherited disease is Choroideremia. In some embodiments, the genetically inherited disease is Focal epilepsy. In some embodiments, the genetically inherited disease is Alpha-1 Antitrypsin deficiency (AATD). In some embodiments, the genetically inherited disease is Androgen Insensitivity Syndrome. In some embodiments, the genetically inherited disease is Opioid-induced hyperalgesia (Target: Raf-1). In some embodiments, the genetically inherited disease is Neurofibromatosis type 1. In some embodiments, the genetically inherited disease is Stargardt's Disease. In some embodiments, the genetically inherited disease is Dravet Syndrome. In some embodiments, the genetically inherited disease is Retinitis Pigmentosa. In some embodiments, the genetically inherited disease is Hemophilia A (factor VIII). In some embodiments, the genetically inherited disease is Hemophilia B (factor IX). In some embodiments, the genetically inherited disease is Parkinson's Disease.

### Linkers

In some embodiments, the linker is a polypeptide linker. In some embodiments, the linker is a non-peptide linker. In some embodiments, the linker comprises a polypeptide portion and a non-peptide portion. In some embodiments, the linker comprises an extended recombinant polypeptide (XTEN). In some embodiments, the linker comprises the amino acid sequence (Gly₄Ser)ₙ (SEQ ID NO: 33380), where n is an integer. In some embodiments, the linker comprises the amino acid sequence (Gly₄Ser)ₙ, wherein n is 1, 2, 3, 4, 5, 6, 7, 8, 9, or 10 (SEQ ID NO: 33381). In some embodiments, the linker comprises the amino acid sequence (Gly₄Ser)ₙ, wherein n is greater than 10 (SEQ ID NO: 33382). In some embodiments, the linker comprises a synthetic portion. In some embodiments, the linker comprises polyethylene glycol (PEG). In some embodiments, the linker is a synthetic linker. In some embodiments (Gly₂Ser)ₙ, wherein n is an integer. In some embodiments, the linker comprises the amino acid sequence (Gly₂Ser)ₙ, wherein n is 1, 2, 3, 4, 5, 6, 7, 8, 9, or 10 (SEQ ID NO: 33383). In some embodiments, the linker comprises the amino acid sequence (Gly₂Ser)ₙ, wherein n is greater than 10 (SEQ ID NO: 33384). In some embodiments, the linker comprises the amino acid sequence (Ser-Gly-Gly-Ser)ₙ (SEQ ID NO: 33385), where n is an integer. In some embodiments, the linker comprises the amino acid sequence (Ser-Gly-Gly-Ser)ₙ, wherein n is 1, 2, 3, 4, 5, 6, 7, 8, 9, or 10 (SEQ ID NO: 33386). In some embodiments, the linker comprises the amino acid sequence (Ser-Gly-Gly-Ser)ₙ, wherein n is greater than 10 (SEQ ID NO: 33387). In some embodiments the linker comprises the amino acid sequence (Glu-Ala-Ala-Ala-Lys)ₙ (SEQ ID NO: 33388), wherein n is an integer. In some embodiments, the linker comprises the amino acid sequence (Glu-Ala-Ala-Ala-Lys)ₙ, wherein n is 1, 2, 3, 4, 5, 6, 7, 8, 9, or 10 (SEQ ID NO: 33389). In some embodiments, the linker comprises the amino acid sequence (Glu-Ala-Ala-Ala-Lys)ₙ, wherein n is greater than 10 (SEQ ID NO: 33390). In some embodiments, the linker comprises a proline linker.

### Methods of the Disclosure

The present disclosure relates to a method of editing a genome using a genome editing system. The present disclosure also relates to the method of editing a genome using a genome editing system, wherein the genome editing system comprises i) an R2 element enzyme, and ii) a payload RNA; wherein the payload RNA comprises one or more of a 5′ homology region, a 3′ homology region, a protein binding element, and an insertion region; wherein the insertion region comprises a template for a small or large nucleic acid insertion into the genome; and wherein the R2 element enzyme comprises a targeting domain, a reverse transcriptase domain, and a nickase domain.

In some embodiments, the target genome is in a eukaryotic cell. In some embodiments, the targeted genome is in a mammalian cell. In some embodiments, the targeted genome is in a dividing mammalian cell. In some embodiments, the targeted genome is in a non-dividing cell. In some embodiments, the targeted genome is in a quiescent cell.

In some embodiments, the genome editing system targets a genomic position for deletion rather than editing. In some embodiments, the genome editing system targets a genomic site for deletion that is between 1 and 150 nucleotides. In some embodiments, the genome editing system comprises a payload RNA with a 5′ homology region and a 3′ homology region, wherein the 5′ homology region and the 3′ homology region, wherein the 5′ homology region and the 3′ homology region are positioned to delete the genomic target. In some embodiments, the genome editing system is capable of deleting a genomic target and inserting a novel nucleic acid region into the genome concurrently.

### Compositions

The present disclosure relates to compositions, wherein the composition comprises a cell, and wherein the cell comprises a genome that has been edited using a genome editing system.

### Sequences

Exemplary sequences of payload UTRs and target homologies are provided in Table 1.

TABLE 1
Exemplary payload UTRs and target homologies.

| | |
|---|---|
| Name | Sequence |

28S 5’ sequence

| | |
|---|---|
| 50 bp | ATTCAATGAAGCGCGGGTAAACGGCGGGAGTAACTATGAC |
| | TCTCTTAAGG (SEQ ID NO: 33391) |
| 30 bp | ACGGCGGGAGTAACTATGACTCTCTTAAGG (SEQ ID NO: |
| | 33392) |

5’ UTRs

| | |
|---|---|
| Full length 5’ UTR | GTCTAGTTACAACTGGGCATCGCTGCAGAGATCGCACCTC |
| R2Tg | CTCGTGGTCCCGCTGGTAGCCCTTCGAAGGGTGACTAAGT |
| | CGATCTCTGCCCCAGGTACGGAGCCGTTGGGACTCACCAG |
| | TCCAACGTAACTCCTGCCTAAATTCGGTGAAACAAATTCCT |
| | CGGTAAAAAGCCCC (SEQ ID NO: 33393) |
| truncated 5’ UTR | GTCTAGTTACAACTG (SEQ ID NO: 33394) |
| R2Tg | |
| Full length 5’ UTR | TCTAGTTACAACTGGGCATAGCTGCAGAGATCTCACCTCCT |
| R2Toc | CGTGGTCCCGCTGGTAAGCCCTTAACAGGGTGACTAAGTA |
| | GATCTCTGCCCCAGTCAAGGAGCCGCTGGGAATCACCAGC |
| | CCAGCGATTCCTTTCAAATTTAGGTGAAACAAATTTCTCGG |
| | TGTGGGTCGCAAGACTTACTACCTAAAACCTGGCCCCACG |
| | GTCTGACAGGGGCAACGGGTTCGGAGAT (SEQ ID NO: |
| | 33395) |
| truncated 5’ UTR | TCTAGTTACAACTGG (SEQ ID NO: 33396) |
| R2Toc | |
| Full length 5’ UTR | TCGGCGATGCTGAACCACCTCCTCGTGGTGCCGACTGGGC |
| nLTR1Mbr | AGCTTTGGAGAAATCCTAAGCTGGCTAAGAGTTCAGCAAC |
| | TCCTG (SEQ ID NO: 33397) |
| 28S/5' UTR truncation | gttgacgcgatgtgatttctgcccagtgctctgaatgtcaaagtgaagaaattcaatgaagcgcg |
| 1 | (SEQ ID NO: 33398) |
| 28S/5' UTR truncation | attcaatgaagcgcgggtaaacggcgggagtaactatgactctcttaaggtctagttacaactgg |
| 2 | (SEQ ID NO: 33399) |
| 28S/5' UTR truncation | atgactctcttaaggtctagttacaactgggcatcgctgcagagatcgcacctcctcgtggtccc |
| 3 | (SEQ ID NO: 33400) |
| 28S/5’ UTR truncation | cgctgcagagatcgcacctcctcgtggtcccgctggtagcccttcgaagggtgactaagtcgatc |
| 4 | (SEQ ID NO: 33401) |
| 28S/5' UTR truncation | gtagcccttcgaagggtgactaagtcgatctctgccccaggtacggagccgttgggactcacca |
| 5 | g (SEQ ID NO: 33402) |
| 28S/5' UTR truncation | cccaggtacggagccgttgggactcaccagtccaacgtaactcctgcctaaattcggtgaaacaa |
| 6 | (SEQ ID NO: 33403) |
| 28S/5' UTR truncation | gactcaccagtccaacgtaactcctgcctaaattcggtgaaacaaattcctcggtaaaaagcccc |
| 7 | (SEQ ID NO: 33404) |

Target homologies

| | |
|---|---|
| AAVS1 cargo 100 bp | AGGGCCGGTTAATGTGGCTCTGGTTCTGGGTACTTTTATCT |
| 5’ homology | GTCCCCTCCACCCCACAGTGGGGCCACTAGGGACAGGATT |
| | GGTGACAGAAAAGCCCCAT (SEQ ID NO: 33405) |
| AAVS1 cargo 100 bp | CCTTAGGCCTCCTCCTTCCTAGTCTCCTGATATTGGGTCTA |
| 3’ homology | ACCCCCACCTCCTGTTAGGCAGATTCCTTATCTGGTGACAC |
| | ACCCCCATTTCCTGGAGC (SEQ ID NO: 33406) |
| NOLC1 cargo 50 bp 5’ | TCCTGAGTCGTGCTGCGTCGACAACGGTAGTGACGCGTAT |
| homology | TGCCTGGAGG (SEQ ID NO: 33407) |
| NOLC1 cargo 50 bp 3’ | GCGGACGCCGGCATTCGCCGCGTGGTTCCCAGCGACCTGT |
| homology | ATCCCCTCGT (SEQ ID NO: 33408) |
| LMNB1 cargo 50 bp 5’ | GCCATGGCGACTGCGACCCCCGTGCCGCCGCGGATGGGCA |
| homology | GCCGCGCTGG (SEQ ID NO: 33409) |
| LMNB1 cargo 50 bp 3’ | CGGCCCCACCACGCCGCTGAGCCCCACGCGCCTGTCGCGG |
| homology | CTCCAGGAGA (SEQ ID NO: 33410) |
| EMX1 cargo 50 bp 5’ | GAGGACATCGATGTCACCTCCAATGACTAGGGTGGGCAAC |
| homology | CACAAACCCA (SEQ ID NO: 33411) |
| EMX1 cargo 50 bp 3’ | CGAGGGCAGAGTGCTGCTTGCTGCTGGCCAGGCCCCTGCG |
| homology | TGGGCCCAAG (SEQ ID NO: 33412) |

Exemplary sequences of Cas9 guides are provided in Table 2.

TABLE 2
Exemplary Cas9 guides

| Name | Sequence | SEQ ID NO: |
|---|---|---|
| AAVS1_guide_1 A1 | gAAGGAGGAGGCCTAAGGATG | 33413 |
| AAVS1_guide_2 A2 | gCTGTCCCCTCCACCCCACAG | 33414 |
| AAVS1_guide_3 A3 | gATATCAGGAGACTAGGAAGG | 33415 |
| AAVS1_guide_4 A4 | gAGGGCCGGTTAATGTGGCTC | 33416 |
| AAVS1_guide_5 A5 | gCTAGTGGCCCCACTGTGGGG | 33417 |
| AAVS1_guide_6 A6 | GAAGGAGGAGGCCTAAGGAT | 33418 |
| AAVS1_guide_7 A7 | GGAAGGAGGAGGCCTAAGGA | 33419 |
| AAVS1_guide_8 A8 | GTCCCCTCCACCCCACAGTG | 33420 |
| AAVS1_guide_9 A9 | gACTAGGAAGGAGGAGGCCTA | 33421 |
| NOLC1_guide_1 N1 | GAGTCGTGCTGCGTCGACAA | 33422 |
| NOLC1_guide_2 N2 | gCGGTAGTGACGCGTATTGCC | 33423 |
| NOLC1_guide_3 N3 | gTAGTGACGCGTATTGCCTGG | 33424 |
| NOLC1_guide_4 N4 | GACGCGTATTGCCTGGAGGA | 33425 |
| NOLC1_guide_5 N5 | GCGTATTGCCTGGAGGATGG | 33426 |
| NOLC1_guide_6 N6 | GCCTGGAGGATGGCGGACGC | 33427 |
| NOLC1_guide_7 N7 | GCCGGCGTCCGCCATCCTCC | 33428 |
| NOLC1_guide_8 N8 | GGGAACCACGCGGCGAATGC | 33429 |
| NOLC1_guide_9 N9 | gACAGGTCGCTGGGAACCACG | 33430 |
| NOLC1_guide_10 N10 | gACGAGGGGATACAGGTCGCT | 33431 |
| NOLC1_guide_11 N11 | gCACGAGGGGATACAGGTCGC | 33432 |
| NOLC1_guide_12 N12 | gAGCCGAGCACGAGGGGATAC | 33433 |
| LMNB1 g1 | GCTGTCTCCGCCGCCCGCCA | 33434 |
| LMNB1 g2 | gCTGCGACCCCCGTGCCGCCG | 33435 |
| LMNB1 g3 | GACCCCCGTGCCGCCGCGGA | 33436 |
| LMNB1 g4 | gACCCCCGTGCCGCCGCGGAT | 33437 |
| LMNB1 g5 | gCCGCGGATGGGCAGCCGCGC | 33438 |
| LMNB1 g6 | gCGGATGGGCAGCCGCGCTGG | 33439 |
| LMNB1 g7 | gTGGGGCTCAGCGGCGTGGTG | 33440 |
| LMNB1 g8 | gCGTGGGGCTCAGCGGCGTGG | 33441 |
| LMNB1 g9 | GACAGGCGCGTGGGGCTCAG | 33442 |
| LMNB1 g10 | GGAGCCGCGACAGGCGCGTG | 33443 |
| LMNB1 g11 | gTGGAGCCGCGACAGGCGCGT | 33444 |
| LMNB1 g12 | gCCTTCTCCTGGAGCCGCGAC | 33445 |
| EMX1 g1 | GGGCAACCACAAACCCACGA | 33446 |
| EMX1 g2 | gAAGCAGCACTCTGCCCTCGT | 33447 |
| EMX1 g3 | gCAAGCAGCACTCTGCCCTCG | 33448 |
| EMX1 g4 | gCTTGGGCCCACGCAGGGGCC | 33449 |
| EMX1 g5 | gTCCAGCTTGGGCCCACGCAG | 33450 |
| EMX1 g6 | GTCCAGCTTGGGCCCACGCA | 33451 |
| EMX1 g7 | GAGTGGCCAGAGTCCAGCTT | 33452 |

Exemplary sequences of NGS, gel primers, and Sanger primers are provided in Table 3.

TABLE 3
Exemplary NGS, gel primers, and Sanger primers.

| Name | Sequence | SEQ ID NO: |
|---|---|---|
| GLuc cargo reporter 28S 5′ | ACACTCTTTCCCTACACGACGCTCT | 33453 |
| junction stagger 1 Forward | TCCGATCTCCAGGTAAGTATCAAGG | |
| | TTACAAGACAGG | |
| GLuc cargo reporter 28S 5′ | ACACTCTTTCCCTACACGACGCTCT | 33454 |
| junction stagger 2 Forward | TCCGATCTACCAGGTAAGTATCAAG | |
| | GTTACAAGACAGG | |
| GLuc cargo reporter 28S 5′ | ACACTCTTTCCCTACACGACGCTCT | 33455 |
| junction stagger 3 Forward | TCCGATCTGACCAGGTAAGTATCAA | |
| | GGTTACAAGACAGG | |
| GLuc cargo reporter 28S 5′ | ACACTCTTTCCCTACACGACGCTCT | 33456 |
| junction stagger 4 Forward | TCCGATCTTGACCAGGTAAGTATCA | |
| | AGGTTACAAGACAGG | |
| GLuc cargo reporter 28S 5′ | GTGACTGGAGTTCAGACGTGTGCTC | 33457 |
| junction in 5′ Tg UTR | TTCCGATCTCTGGTGAGTCCCAACG | |
| Reverse | GCTC | |
| GLuc cargo reporter 28S 5′ | GTGACTGGAGTTCAGACGTGTGCTC | 33458 |
| junction scarless Reverse | TTCCGATCTCACAGATCGACCTGTG | |
| | GAGAGAAAG | |
| GLuc cargo reporter 28S 5′ | GTGACTGGAGTTCAGACGTGTGCTC | 33459 |
| junction non-inserted | TTCCGATCTGAGGGATCTGCGGCCG | |
| Reverse | CTT | |
| Genomic AAVS1 5′ junction | ACACTCTTTCCCTACACGACGCTCT | 33460 |
| Forward | TCCGATCTCCGAGCTGGGACCACCT | |
| | TATATTC | |
| Genomic AAVS1 5′ junction | GTGACTGGAGTTCAGACGTGTGCTC | 33461 |
| scarless Reverse | TTCCGATCTCGTTGGCAAGCCCTTT | |
| | GAGGCA | |
| Genomic AAVS1 5′ junction | GTGACTGGAGTTCAGACGTGTGCTC | 33462 |
| non -inserted Reverse | TTCCGATCTCCCTCCCAGGATCCTC | |
| | TCTGGC | |
| Genomic NOLC1 5′ junction | ACACTCTTTCCCTACACGACGCTCT | 33463 |
| Forward | TCCGATCTCAATGACGTAACACAGG | |
| | CCCGC | |
| Genomic NOLC1 5′ junction | GTGACTGGAGTTCAGACGTGTGCTC | 33464 |
| scarless Reverse | TTCCGATCTTCCTGTCGCTTTGGCG | |
| | AACTTATTG | |
| Genomic NOLC1 5′ junction | GTGACTGGAGTTCAGACGTGTGCTC | 33465 |
| non-inserted Reverse | TTCCGATCTCGAGCACGAGGGGATA | |
| | CAGGTC | |
| pMax cargo genomic 28S 5′ | CCCACCCCACGTCTCGTCGCG | 33466 |
| junction gel Forward | | |
| pMax cargo genomic 28S 5′ | CCGAAGTGGTAGAAGCCGTAGC | 33467 |
| junction gel Reverse | | |
| pMax cargo genomic 28S 3′ | GCCCGCACCTTCAGCCTOCGC | 33468 |
| junction gel Forward | | |
| pMax cargo genomic 28S 3′ | TCCGATCTGCCGGGGGCCTCCCACT | 33469 |
| junction gel Reverse | TATT | |
| GLuc cargo reporter 28S 5′ | CCAGGTAAGTATCAAGGTTACAAGA | 33470 |
| junction Forward | CAGG | |
| GLuc cargo reporter 28S 5′ | CCACCTGGCCCTGGATOTTGCTGGC | 33471 |
| junction Reverse | AAAG | |
| GLuc cargo reporter 28S 3′ | TAAGGAGACCAATAGAAACTGGGCT | 33472 |
| junction Forward | TGTCGAGACAGAGAAG | |
| GLuc cargo reporter 28S 3′ | CACCGGCCTTATTCCAAGCGGCTTC | 33473 |
| junction Reverse | GGC | |

Exemplary sequences of ddPCR primers and probes are provided in Table 4.

TABLE 4
Exemplary ddPCR primers and probes.

| | | SEQ ID |
|---|---|---|
| Name | Sequence | NO: |
| NOLC1 Endogenous locus | TGGAGCCCACCCTTTCCGT | 33474 |
| ddPCR Forward | | |
| LMNB1 Endogenous locus | TCCTTATCACGGTCCCGCT | 33475 |
| ddPCR Forward | CG | |
| EMX1 Endogenous locus | GCATTGCCACGAAGCAGG | 33476 |
| ddPCR Forward | | |
| EGFP R2 cargo ddPCR | GAACTCCACGCCGTTCA | 33477 |
| Reverse | | |
| EGFP R2 cargo ddPCR | /56-FAM/CC ATG AAG | 33478 |
| FAM probe | A/ZEN/T CGA GTG CCG | |
| | CAT CA/3IABkFQ/ | |

Having now described the present disclosure in detail, the same will be more clearly understood by reference to the following examples. The following examples are included solely for purposes of illustration and are not considered limiting embodiments. All patents and publications referred to herein are expressly incorporated by reference.

### EXAMPLES

### Example 1. Insertion of Non-Human R2 into the 28S Locus of the Human Genome

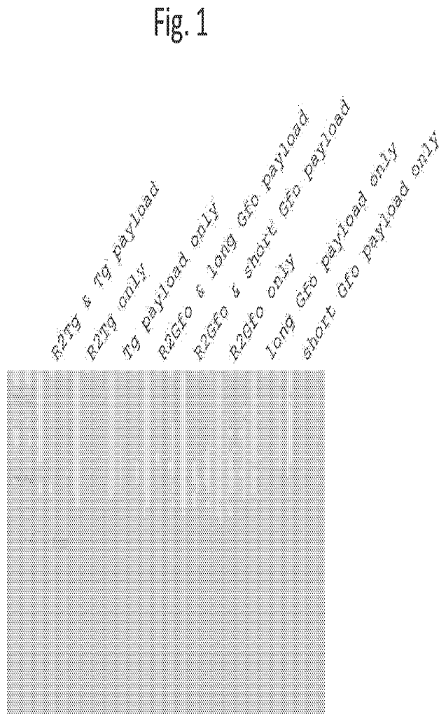

To determine the ability of animal R2 elements to integrate into the human genome, HEK293FT cells were transfected with specific plasmids containing the zebra finch (Taeniopygia guttata) R2 element (R2Tg), a payload, or both the R2tg plasmid and a payload plasmid. Following isolation of DNA from transfected cells, those cells transfected with an R2Tg plasmid and an eGFP payload (eGFP flanked by UTR regions and 100 bp homology to the human R2 locus), showed a distinct PCR product (FIG. 1, lane 1), indicating integration of the eGFP payload into the human genome through R2Tg. When cells were transfected with R2Tg without a payload (FIG. 1, lane 2), payload alone with no R2 (FIG. 1, lanes 3, 8, 9), or other R2 orthologs from Geospiza fortis (Gfo; FIG. 1, lane 3-5) with or without payloads, no PCR product is identified These results demonstrate that R2Tg can successfully integrate payloads into the human genome.

### Example 2. Modification of the Target Landing Site

Following successful insertion of an eGFP payload, the features of the R2 system that could increase integration efficiency were examined. In the following experiments, unless otherwise stated, three plasmids are used. The first plasmid contains at least an R2 protein. The second plasmid contains at least a portion of a payload reporter. The third plasmid contains at least R2 landing sites.

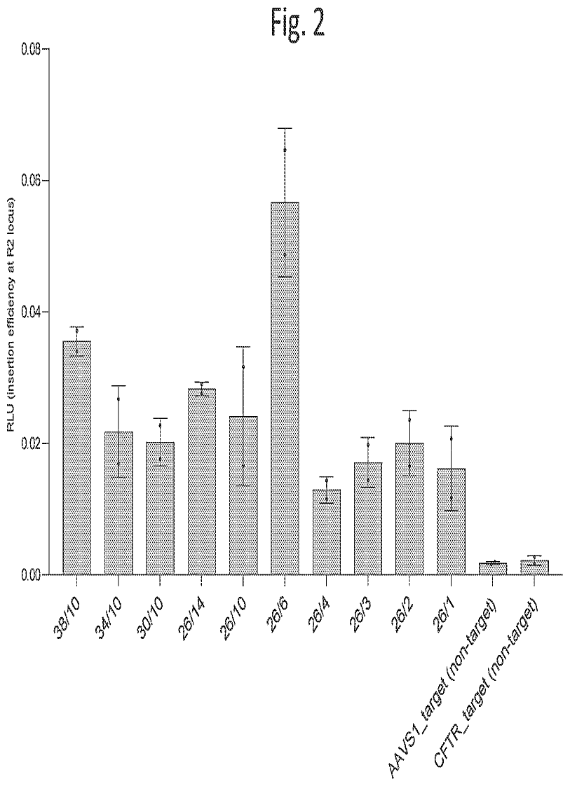

The R2 landing site plasmids contain R2 landing sites of variable size. This size is indicated in the format 26/3 (FIG. 2), where the first number indicates the number of base pairs upstream of the insertion site, and the second number indicates the number of base pairs downstream of the insertion site.

Following transfection of these three plasmids with varying length of R2 landing sites, integration was measured by luminescence, indicating integration of the luminescent payload (FIG. 2). of an artificial luciferase exon (introduced only with the payload) that allows the inserted reporter to splice and reconstitute a functional luciferase gene (FIG. 2). The payload, which is RNA, is transcribed from a DNA (payload plasmid template) where an artificial luciferase exon is flanked by 5′ and 3′ UTRs as and 5′ and 3′ homologies. Two negative controls (FIG. 2, lanes 11-12) exhibited little luminescence. The landing site which proved to be the most efficient for integration was 26/6 (FIG. 2, lane 6; 26 bp upstream, 6 bp downstream of the insertion site). Given that the normal target site at the 28S locus in the human genome is hundreds of base pairs, it is unexpected that the shorter landing sites tested here provided such efficient integration.

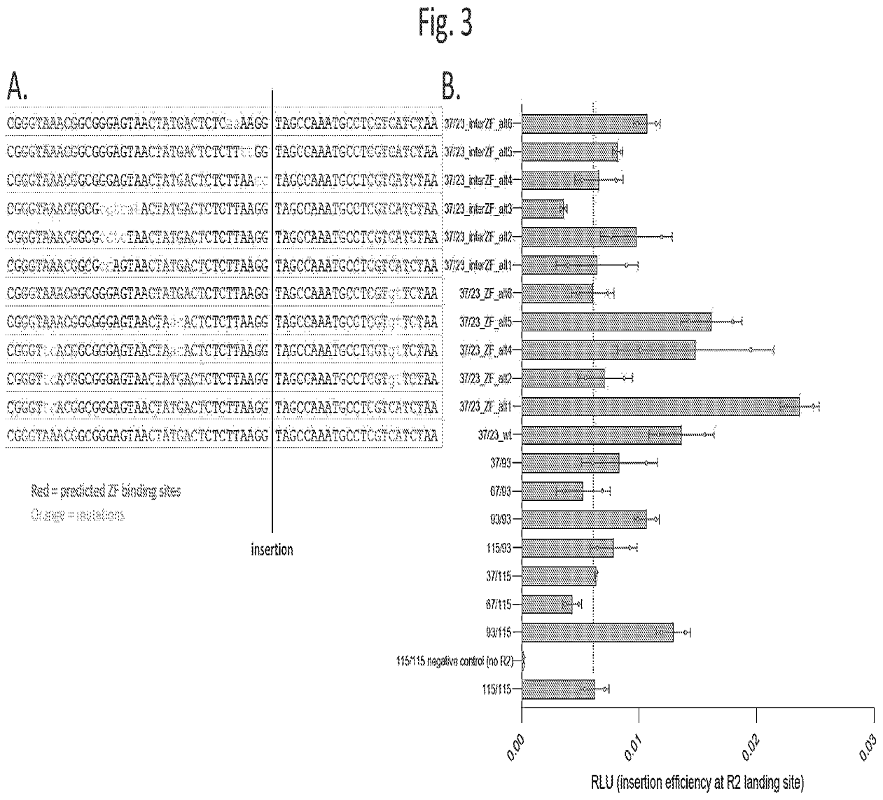

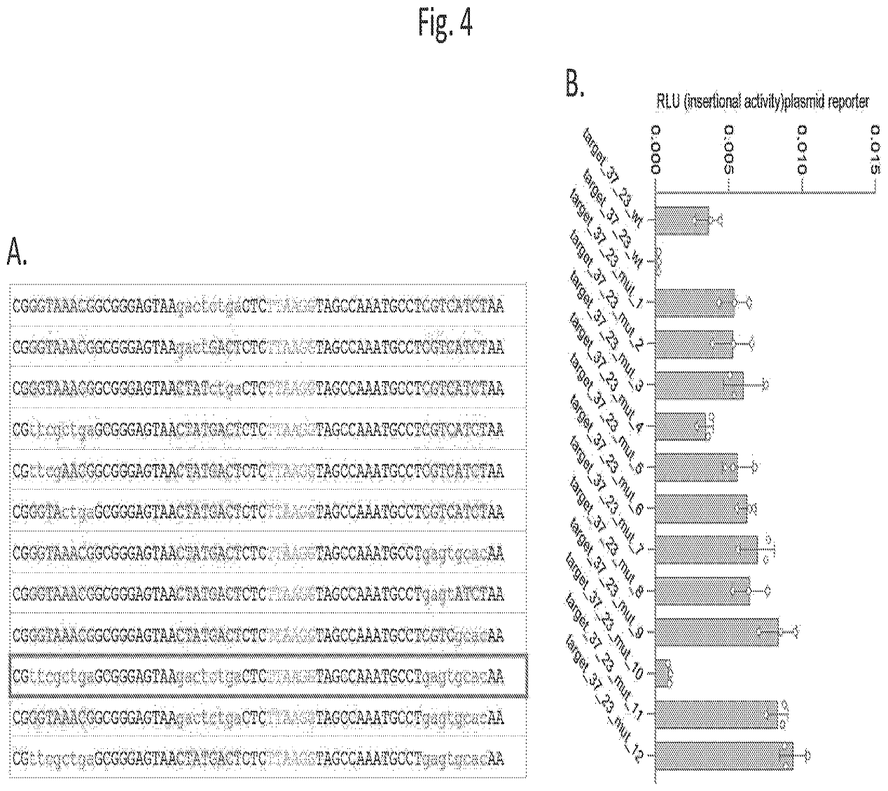

Next, the tolerability of mutations within the R2 landing sites was tested. FIG. 3A displays the predicted zinc finger binding sites (red) within the R2 landing sites and the mutations tested (orange, lowercase bases). FIG. 3 B shows that there is a great deal of tolerability within the R2 landing sites that still allows for integration. FIG. 4 shows additional mutations that may be tolerated. However, mutation of all three, predicted zinc finger binding sites results in abrogated insertion efficiency (FIG. 4B, target_37_23_mut_10). Based on this evidence, a great degree of tolerability for mutations away from the traditional R2 landing sites is found and can help in the development of exogenous landing sites.

### Example 3. Modification of the Payload Homology Regions

After determining that short landing sites could provide for efficient integration, the effect of insertion homology length (to the landing sites) on integration efficiency was evaluated. To test the effect of homology length on integration efficiency, HEK293FT cells were transfected with three separate plasmids. The first plasmid contained an R2 protein encoding region, the second plasmid encoded a partial (inactive) luciferase reporter region and R2 landing sites, and the third plasmid encoded a luciferase insertion as well as regions of homology of varying number of base pairs homologous to the R2 landing site in the second plasmid. Cells were then treated with aphidicolin, which blocks cell division and thus also stops Homology Directed Repair (HDR). Without being bound to any one theory, by blocking HDR, integration is more likely to occur due to an R2 related mechanism.

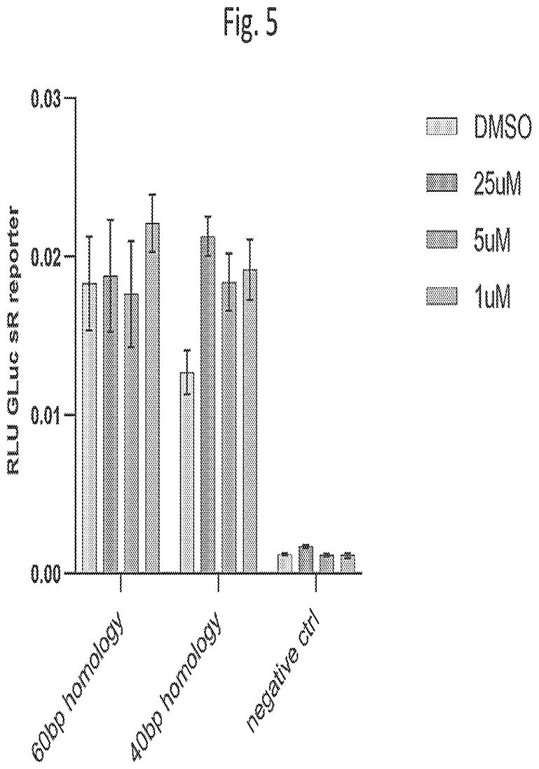

When treated with 1 μm, 5 μm, or 25 μm aphidicolin (or DMSO control) (FIG. 5), plasmids with either 60 base pair homology (FIG. 5, columns 1-4) or 40 base pair homology (FIG. 5, columns 5-8) still exhibited successful integration, indicating that the integration of these payloads occurs by an HDR-independent mechanism.

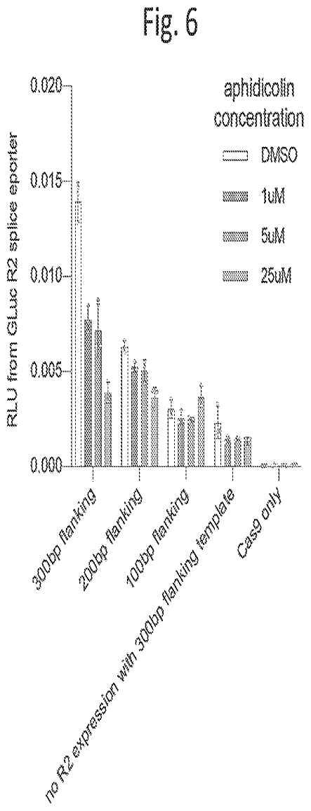

When flanking regions (UTR and additional homology region) were increased in size to 100 bp (FIG. 6, columns 1-4), 200 bp (FIG. 6, columns 5-8), or 300 bp (FIG. 6, columns 9-12) and treated with aphidicolin at 1 μm, 5 μm, or 25 μm (or DMSO control), a significant improvement in integration efficiency is exhibited with longer flanking regions (FIG. 6). When transfected with Cas9 only, no integration was seen. Cells were also transfected with a 300 bp flanking template and no R2 protein (FIG. 6, lanes 13-16) to measure the level of HDR in the system.

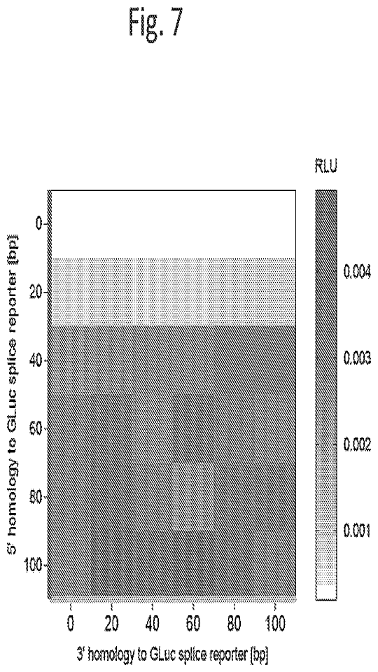

An overview of the role homology of the payload plays in integration efficiency (as measured by luminescent readout) is seen in FIG. 7. Greater 5′ homology (y-axis) to the R2 landing site is associated with more efficient integration. This is not the case for 3′ homology (x-axis), which is less clear, but indicates that shorter homology results in more efficient integration in some cases.

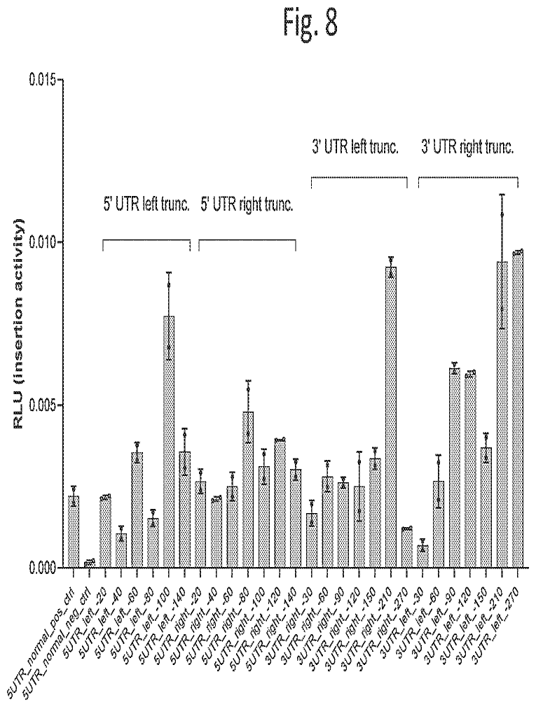

Also, the effect of truncations of the 5′ and 3′UTRs from the payload portion (FIG. 8) on integration efficiency was examined. Three plasmids were transfected into HEK293FT cells. The first plasmid contained a partial luciferase reporter with wild-type R2 landing sites (wtR2) of 26/22 bp. The second plasmid encoded an R2 protein. The third plasmid contained a luciferase payload with the UTR modifications listed along the x-axis. Generally, 3′ UTR (FIG. 8, columns 16-29) truncations resulted in greater integration efficiency (as measured by luminescence readout) than 5′ UTR truncations (FIG. 8, columns 3-15). The greatest increases in integration efficiency were seen in truncations greater than 90 base pairs. Nearly all truncations, however, retained some form of integration activity.

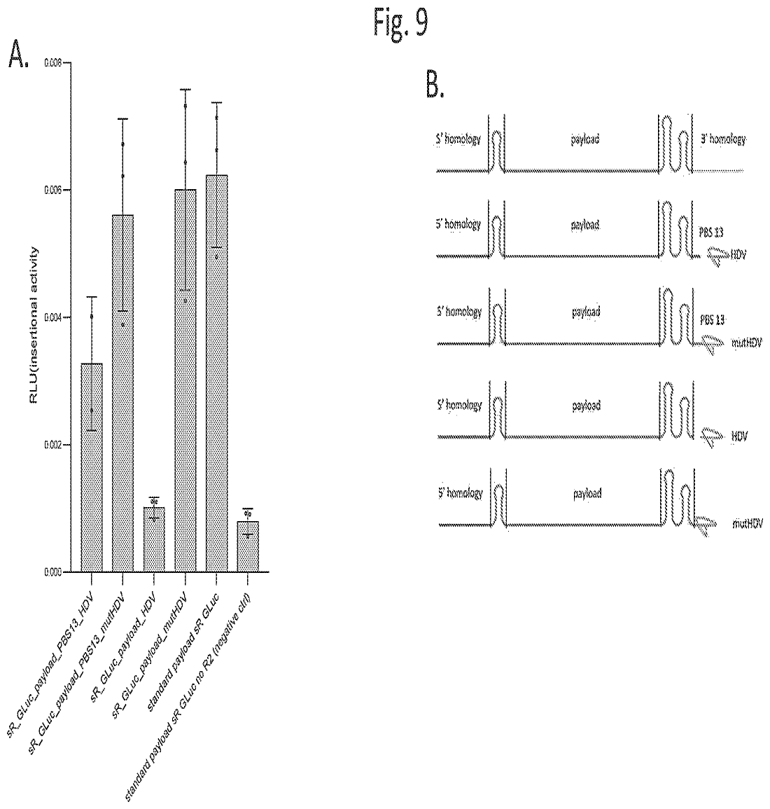

Next, we evaluated how solely altering the 3′ homology regions would affect integration efficiency. In this experiment, HEK293FT cells were transfected with 3 plasmids. The first plasmid contained an R2 protein encoding region. The second plasmid contained a partial luciferase reporter with wtR2 landing sites. The third plasmid contained a luciferase insertion with alterations to the 3′ UTR, as named on the x-axis (FIG. 9A) and described visually in FIG. 9B. HDV is an HDV ribozyme, which cleaves the insertion region directly after the 3′ UTR. mutHDV is an inactive HDV, incapable of cleaving the homology region just beyond the 3′UTR. All modifications retained significant activity, except for the HDV only modification This indicates that cleavage directly beyond the 3′UTR in the homology region (i.e., no further homology region remains), dramatically decreased integration efficiency (FIG. 9A, column 3). This is in concert with the discoveries above, where a minimal (but not absent) 3′ homology region is required for significant integration efficiency.

### Example 4. Modification of the R2 Enzyme

Next, we evaluated whether modifications to the R2 protein could increase integration efficiency. First, permissible domains within the R2 protein into or onto which various additional moieties could be fused were identified. As before, three plasmids were introduced into HEK293FT cells. The first plasmid contained an R2 protein which contained different GFP variants at different points along the R2 protein. The second plasmid encoded a partial (inactive) luciferase reporter region and R2 landing sites, and the third plasmid encoded a luciferase insertion as well as regions of homology to the R2 landing site in the second plasmid.

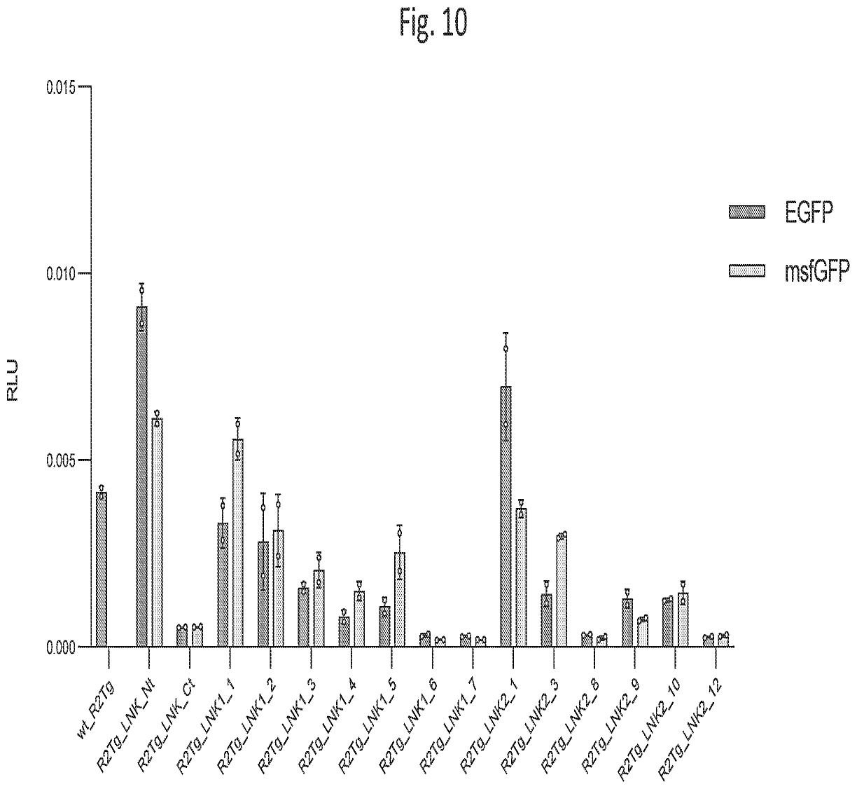

These variant R2 proteins were modified by inserting GFP variants throughout the length of the protein, beginning from the N-terminus. By example, LNK1_1 is located closer to the N-terminus than is LNK1_7. LNK_nt indicates a fusion to the N-terminus, while LNK_ct indicates a fusion to the C-terminus. As seen in FIG. 10, an N-terminal fusion of eGFP resulted in the greatest integration efficiency, suggesting that this fusion may be ideal for additional fusion molecules. However, multiple “permissive insertion sites” were identified in FIG. 10, including R2Tg_LNK1_1, R2Tg_LNK1_2, R2Tg_LNK1_3, R2Tg_LNK1_4, R2Tg_LNK1_5, R2Tg_LNK2_1, R2Tg_LNK2_3, R2Tg_LNK2_9 and R2Tg_LNK2_10 (FIG. 10).

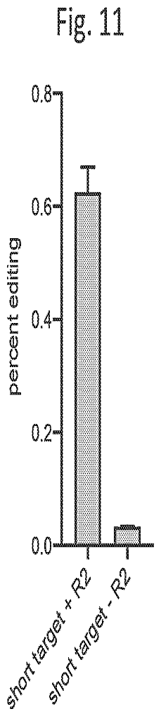

The matter of whether R2 could integrate a payload using a “short target”-truncated R2 landing sites (26/3 bp) was investigated. FIG. 11 exhibits the ability of R2 to deliver a payload even given this short landing site (FIG. 11, column 1).

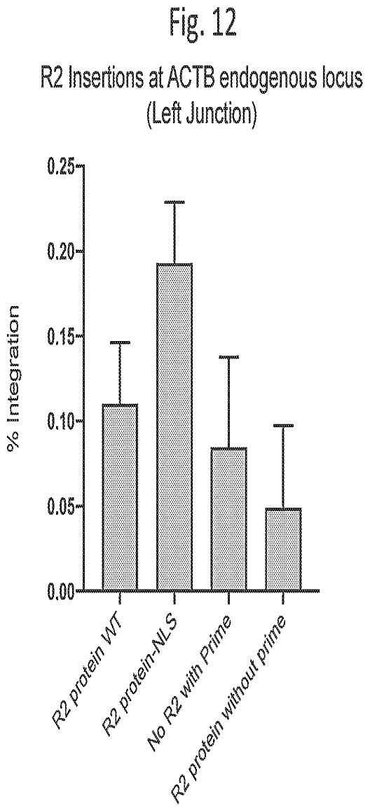

Also, the matter of whether the addition of a nuclear localization signal would increase integration efficiency at the Beta-actin locus of HEK293FT cells (FIG. 12) was examined. HEK293FT cells were transfected with four separate plasmids. The first plasmid encoded an R2 protein. The second plasmid contained pMAX as a payload (including 5′ and 3′ UTRs, as well as 5′ and 3′ homologies) for R2-dependent insertion. The third plasmid encoded a prime editor protein, and the fourth plasmid expressed a prime editing guideRNA. The prime editor first inserts a 48 bp (28S) target site in ACTB to then, in a second step, R2-dependent insertion of the pMAX payload.

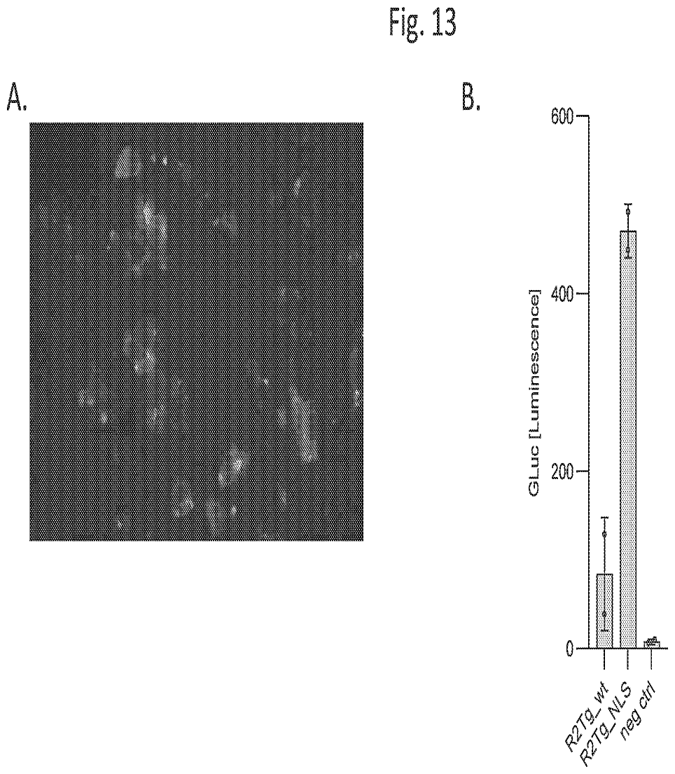

After determining the ability of a nuclear localization signal to boost integration, the primary localization of transfected R2 proteins into HEK293FT cells was evaluated. FIG. 13A shows that R2 does not primarily localize to the nucleus of the cell. However, when HEK293Ft cells were transfected with two plasmids (the first an R2 protein, the second a payload protein) into cells that had been stably transfected to integrate a portion of the splice reporter, addition of a nuclear localization signal to the N- and C-terminus of the R2 protein dramatically increased payload insertion efficiency (FIG. 13B).

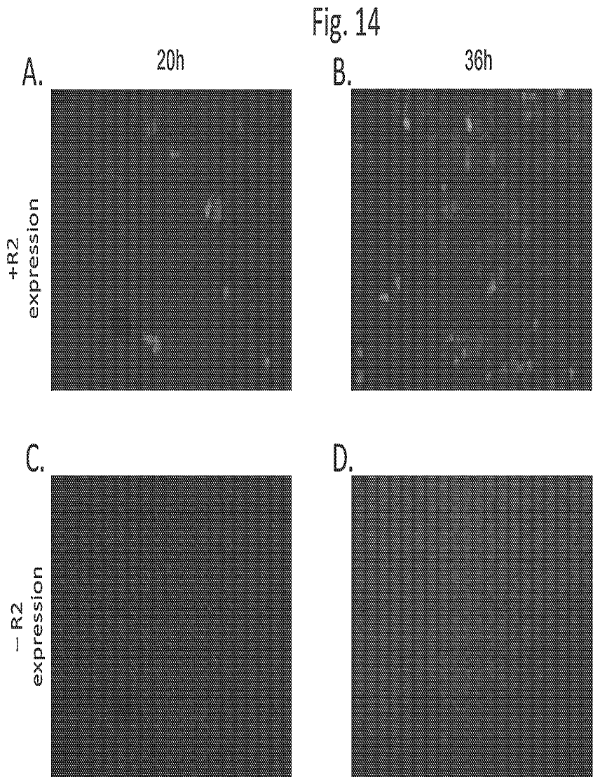

Thus, modifying the R2 protein portion can allow for greater integration efficiency. To further study integration efficiency, a fluorescent GFP reporter responsive to R2 activity (FIG. 14) was developed. The R2 reporter that was developed has a single GFP exon and promoter that is not activated until the R2 payload, with a second GFP exon, is integrated (FIG. 14A, B). Thus, R2 integration can be read by a fluorescent readout.

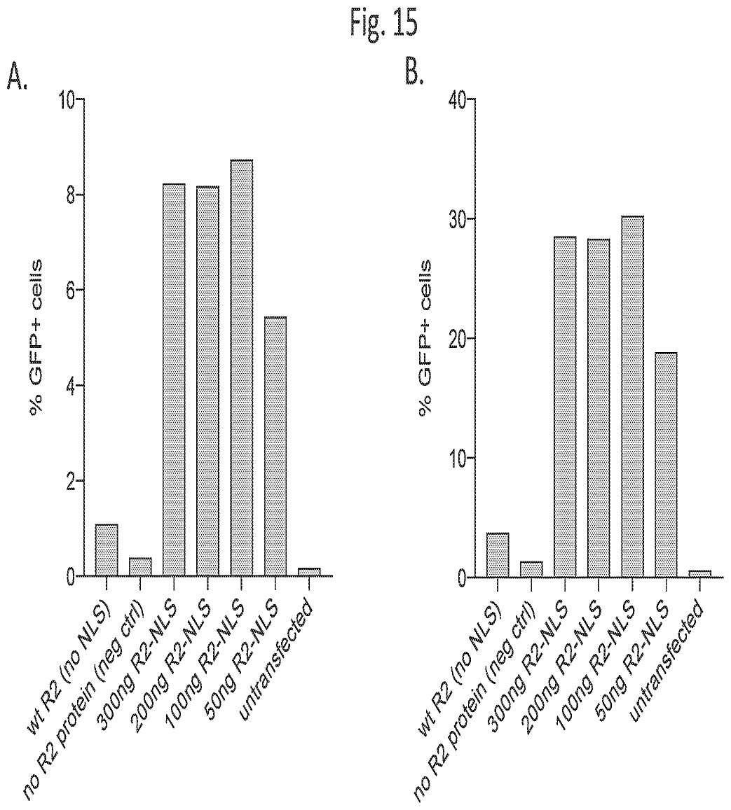

Using this fluorescent readout approach, the efficiency of integration was evaluated using flow cytometry. HEK293FT cells were transfected with specific plasmids. These samples were wild-type R2 (FIG. 15A, column 1), a negative control (FIG. 15A, no R2 protein; column 2), 300 ng of R2 with a nuclear localization signal (FIG. 15A, column 3), 200 ng of R2 with a nuclear localization signal (FIG. 15A, column 4), 100 ng of R2 with a nuclear localization signal (FIG. 15A, column 5), 50 ng of R2 with a nuclear localization signal (FIG. 15A, column 5), and untransfected cells as a percentage of all cells in each sample. The results shown in FIG. 15A clearly demonstrate the increased integration efficiency of R2 proteins with a nuclear localization signal compared to wild type R2 without a nuclear localization signal. This increase persists when the GFP+ cells are normalized to only those cells that were successfully transfected (FIG. 15B).

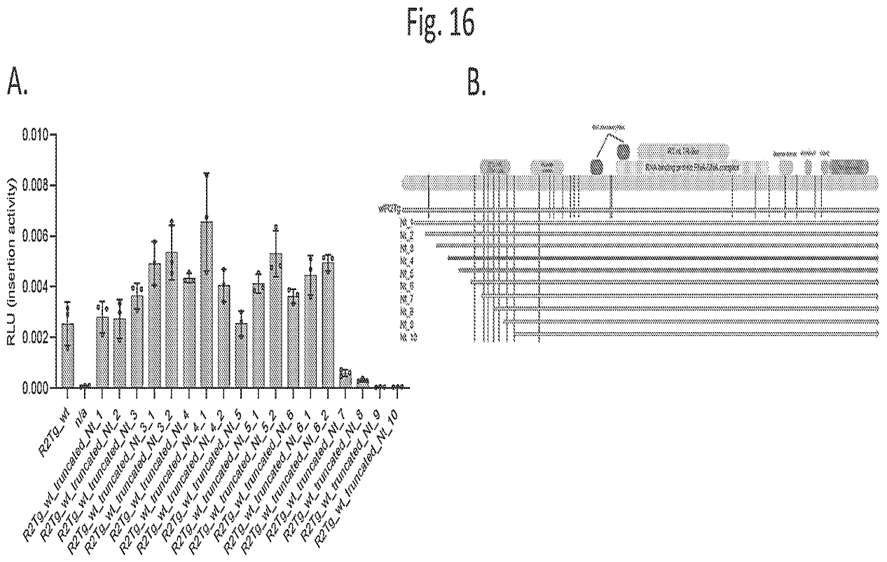

Next, the matter of whether the truncation of the R2 protein resulted in alteration of integration efficiency was studied. The N-terminal portion of the R2 protein was serially truncated, as indicated by the vertical lines in FIG. 16B. These truncations did not result in any significant drop in integration efficiency until reaching NT_7, which demarcates the current limit of truncation for a single R2 protein to maintain integration efficiency.

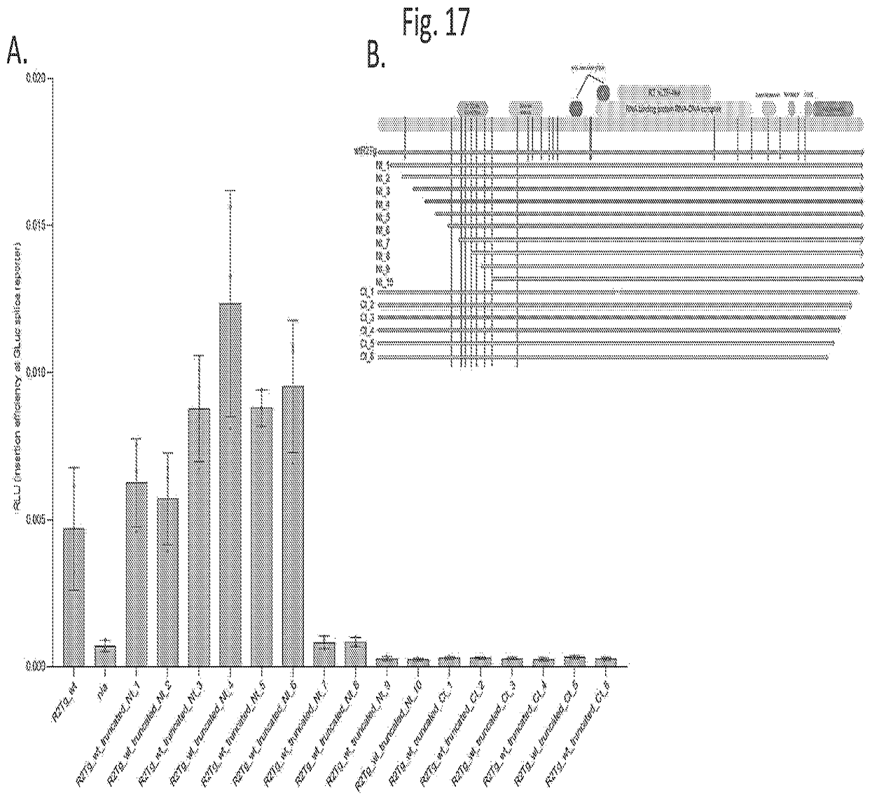

Also, the matter of whether C-terminal truncations of the R2 protein may result in viable R2 proteins that sustain integration efficiency (FIG. 17) was evaluate. However, even the shortest C-terminal truncation resulted in a drastic decrease in integration efficiency, highlighting the importance of the C-terminal domains in contrast to the somewhat expendable N-terminal domains (FIG. 17A, 17B).

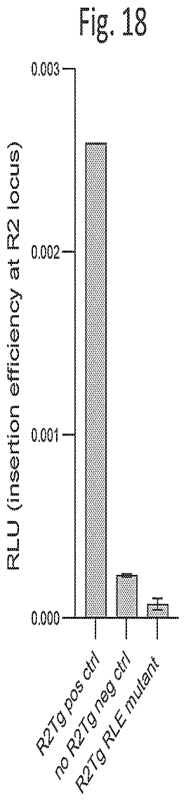

The issue that ablation of the restriction-like endonuclease (RLE) domain would affect integration activity was then studied. HEK293FT cells were transfected by three plasmids. The first plasmid contains a partial luciferase reporter with wtR2 landing sites (26/22 bp). The second plasmid encodes either a wild type R2 protein or an RLE deficient R2 protein. The third plasmid encodes a luciferase payload. Absence of the RLE domain in the R2 protein almost completely abolishes the integration efficiency of a wild-type R2 protein (FIG. 18, column 3).

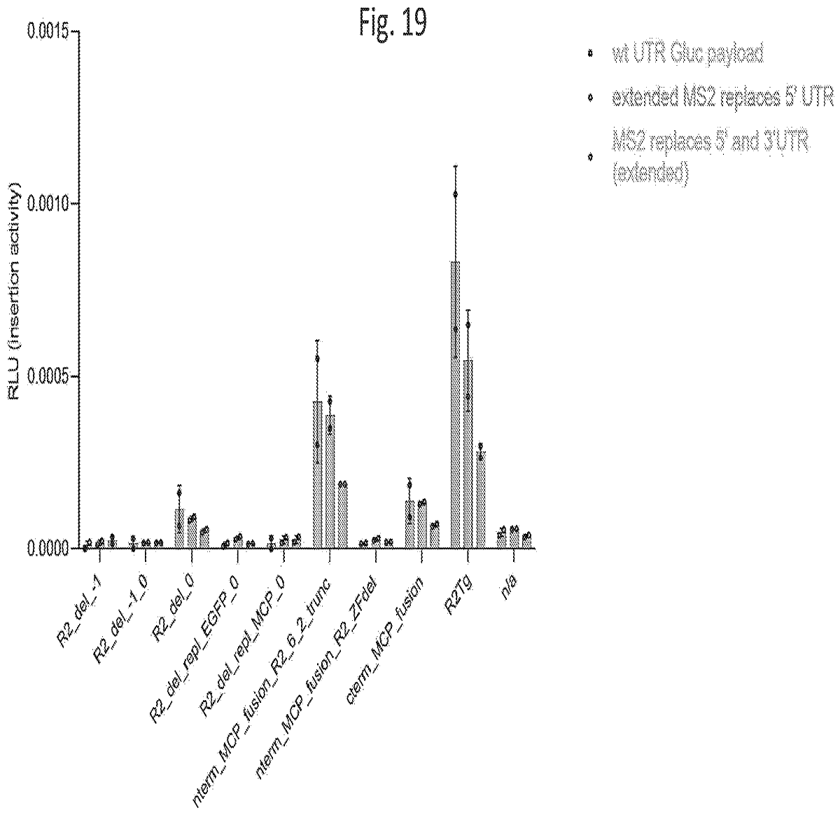

Finally, the matter of whether certain other domains of the R2 protein could be removed or modified without adverse effect. was evaluated. FIG. 19. Displays the results of an experiment in which HEK293FT cells were transfected with 3 plasmids. The first plasmid encoded a partial luciferase reporter with wtR2 landing sites. The second plasmid encoded a luciferase payload. The third plasmid encoded an R2 protein with various modifications, including to the −1 domain, 0 domain, zinc finger domains, or to add C- or N-terminal fusions. Three payloads were examined for each modified group of plasmids. A wild type luciferase payload (orange), a luciferase payload in which the MS2 binding site replaces the 5′UTR, and a luciferase payload in which the 5′ and 3′UTRs are replaced with MS2 binding sites. Deletion of the −1 domain (FIG. 19, columns 1-3), of the −1 and 0 domains (FIG. 19, columns 4-6) and of the 0 domain alone (FIG. 19, columns 7-9) significantly impaired integration efficiency. Further, replacing the 0 domain with an eGFP (FIG. 19, columns 10-12) or with an MCP domain (FIG. 19, columns 13-15) also significantly decrease integration efficiency, as did deleting a zinc finger domain (FIG. 19, columns 19-21). However, fusing the truncated N-terminus of the R2 protein to an MCP domain (i.e., a targeting domain) did show some integration efficiency, though not to the level of wild type. This experiment helps to define the indispensable domains for the R2 protein, and where modifications may be made.

### Example 5. Modification of Payloads

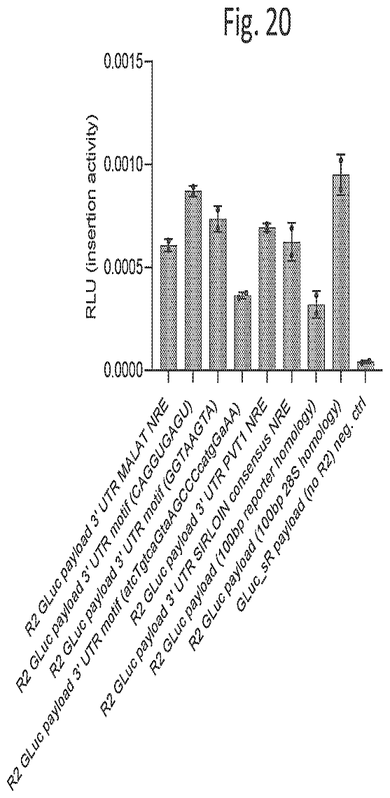

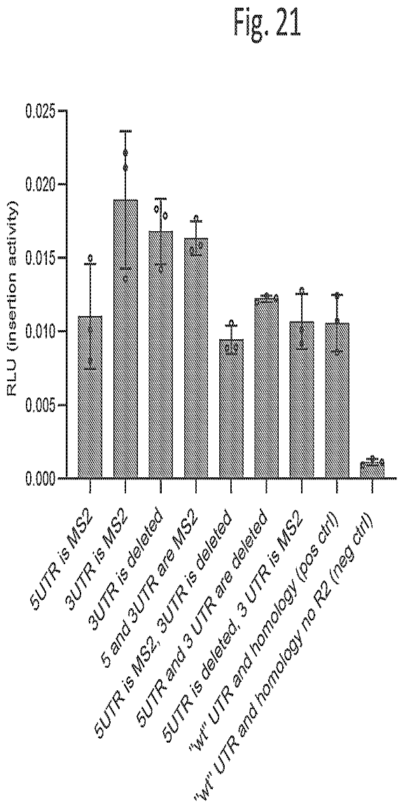

This Example tested whether the payload itself could be modified to sustain nuclear localization. FIG. 20 sets out the relative insertion efficiency of payloads with various nuclear retention elements appended to the payload. Nuclear retention signals have varying levels of effect on the integration efficiency of the R2 payloads, indicating that nuclear localization may be important for optimal integration activity.

Further, whether the UTR elements of the payload were necessary for their integration, or if they may be modified, was studied. In this experiment, HEK293FT cells were transfected with three plasmids. The first plasmid encoded an R2 protein. The second plasmid encoded a partial luciferase reporter and wtR2 landing sites. The third plasmid contained the luciferase payload and any of many UTR modifications (FIG. 21). UTRs were replaced by MS2 binding sites (FIG. 21, columns 1, 2, and 4), the 3′UTR was deleted (FIG. 21, column 3), the 5′ UTR replaced by an MS2 binding site while the 3′UTR is deleted (FIG. 21, column 5), the 5′ and 3′ UTR were both deleted (FIG. 21, column 6), the 5′UTR is deleted and the 3′UTR is replaced with an MS2 binding site (FIG. 21, column 7), as well as positive and negative controls (FIG. 21, columns 8 and 9, respectively). In each situation some integration activity was confirmed. Importantly, this also occurred in which both the 5′ and 3′ UTRs were deleted without any replacement (FIG. 21, column 6).

### Example 6. Linkers and Fusion Proteins

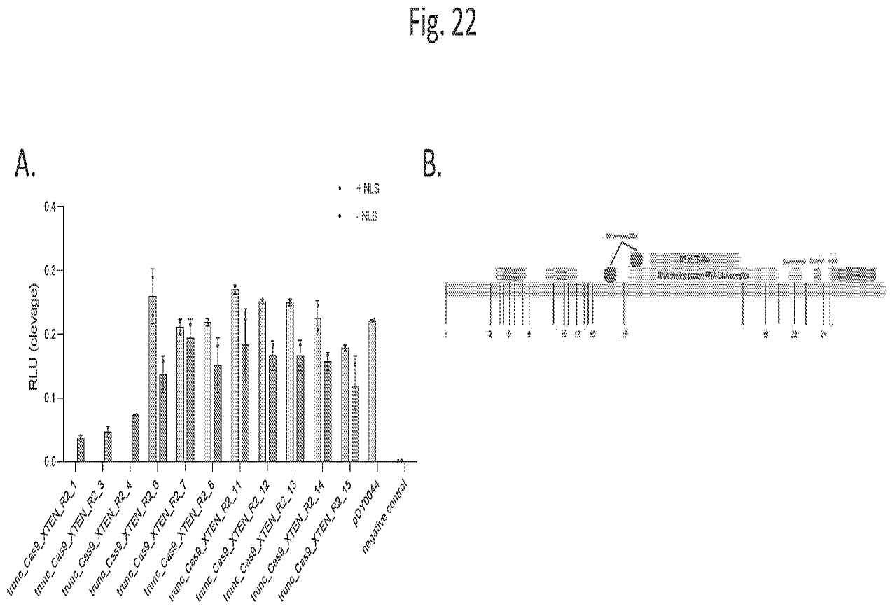

Also, the evaluation of R2 fusion proteins and fusion proteins with linkers were viable for use in genome editing was carried out. In this experiment, HEK293FT cells were transfected with 3 plasmids. The first plasmid contained an R2 protein (with or without an NLS) fused to a Cas9 protein connected by an XTEN linker (16 amino acids in length) at various points through the N-terminal portion of the R2 protein (see FIG. 22B). The second plasmid contains a luciferase reporter that is designed to indicate cleavage by Cas9. The third plasmid expresses a single guide RNA. Multiple Cas9-R2 fusion proteins exhibited the ability to cleave the Cas9 target protein, either with or without the nuclear localization signal (FIG. 22A).

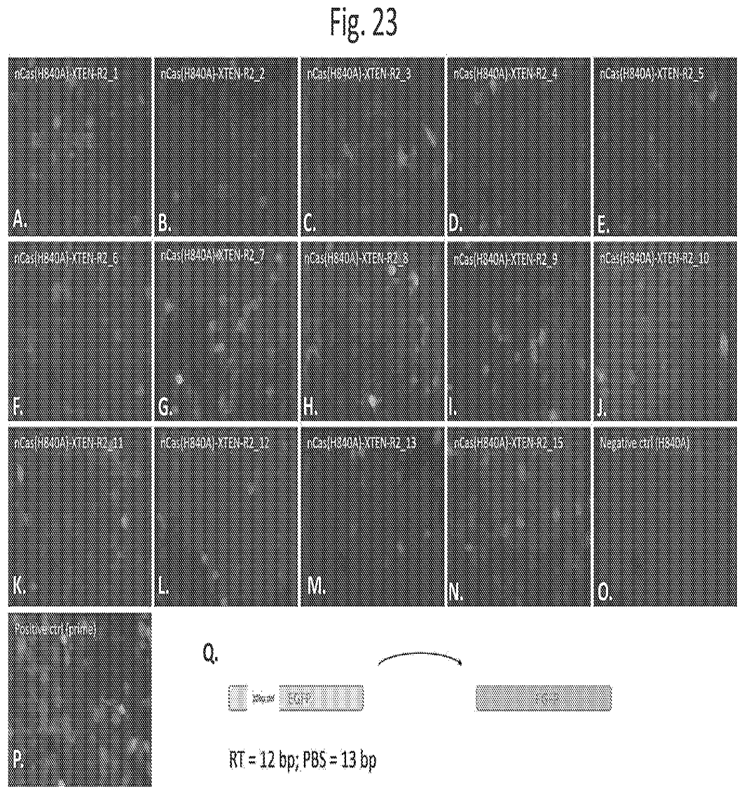

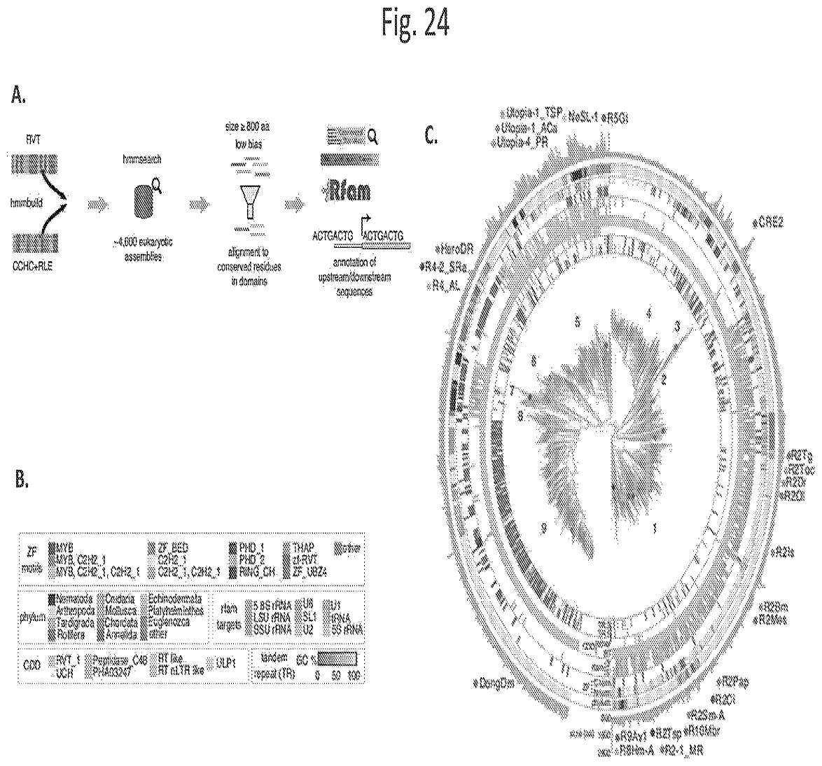

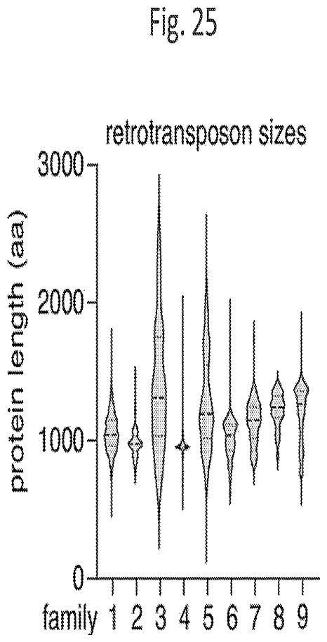

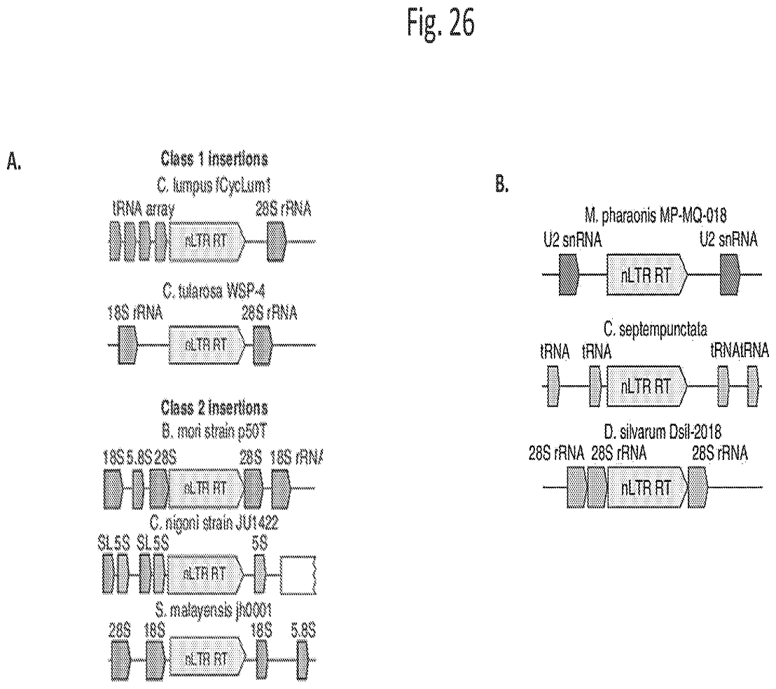

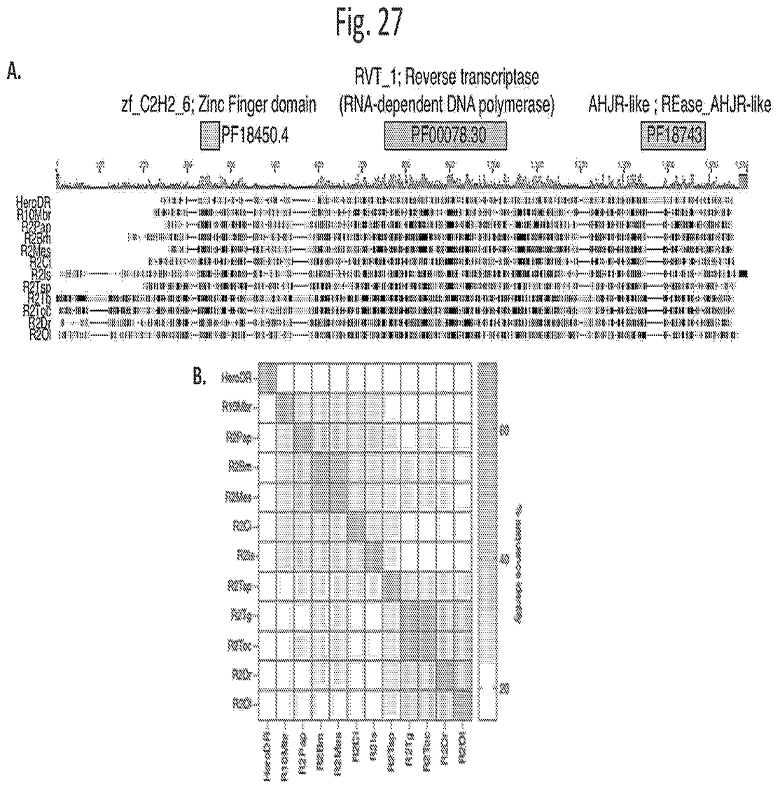

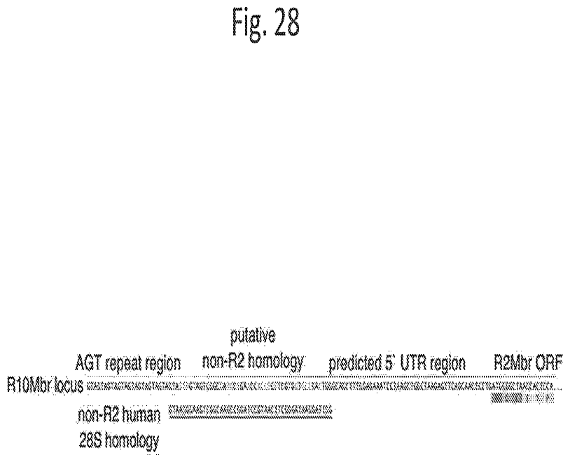

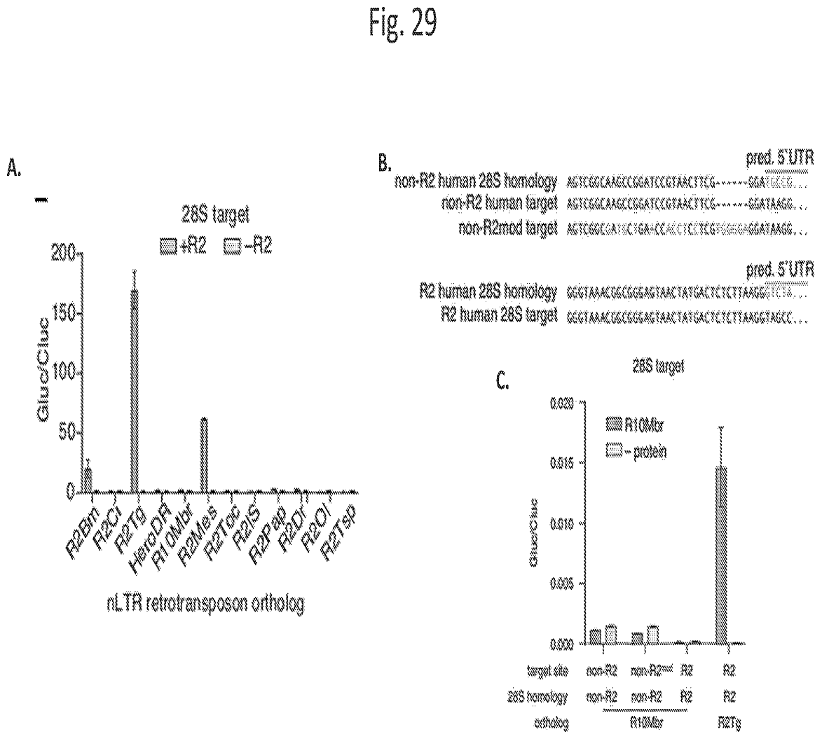

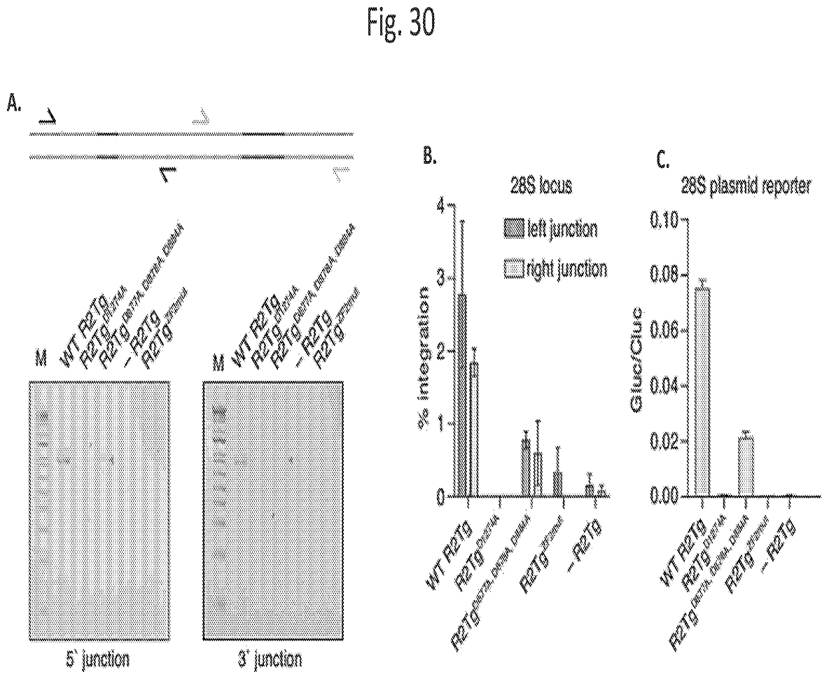

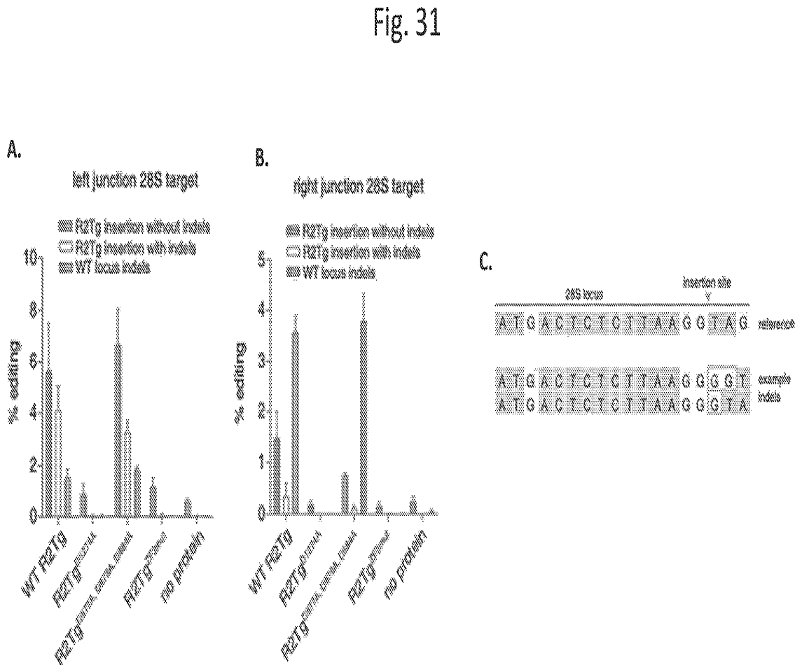

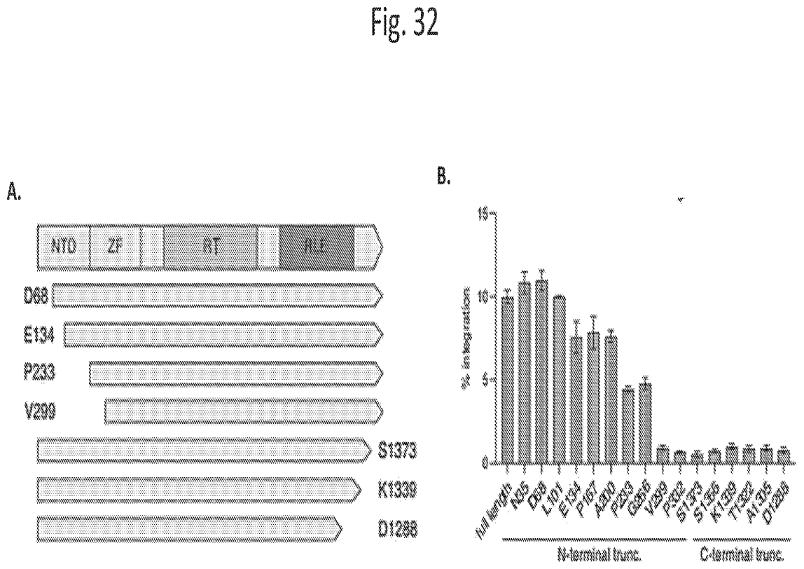

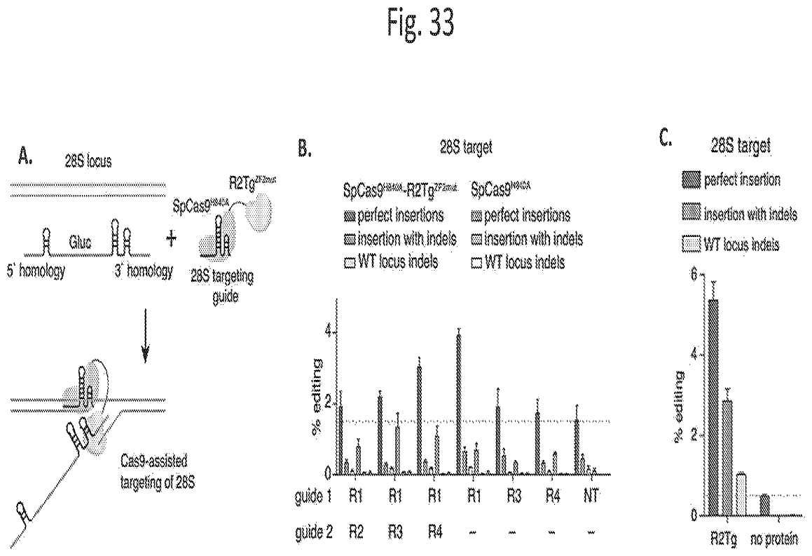

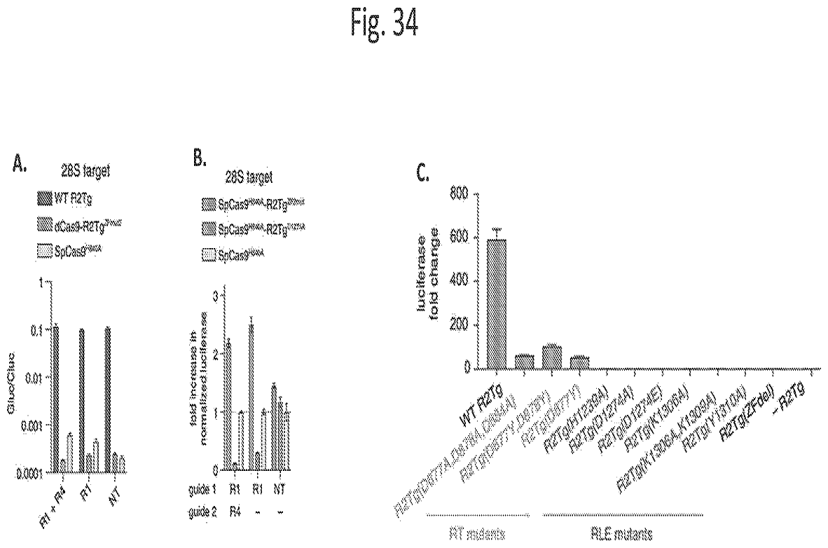

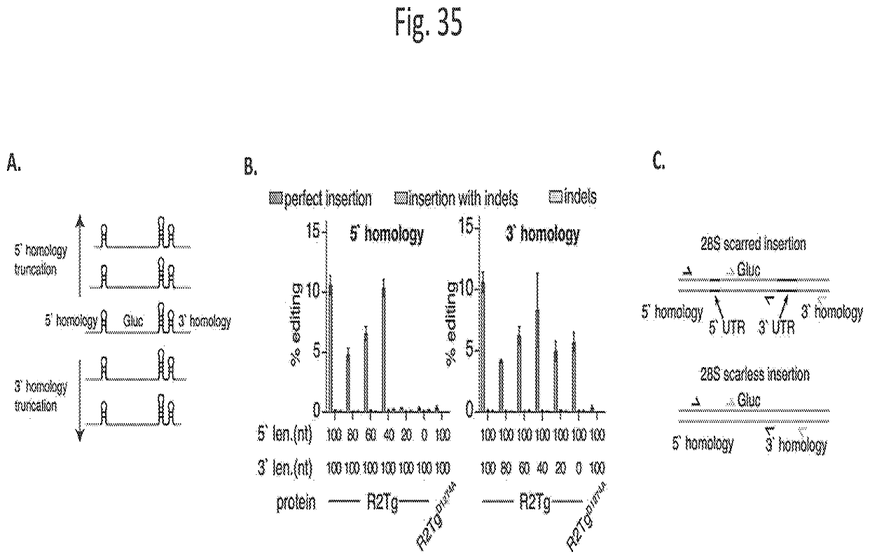

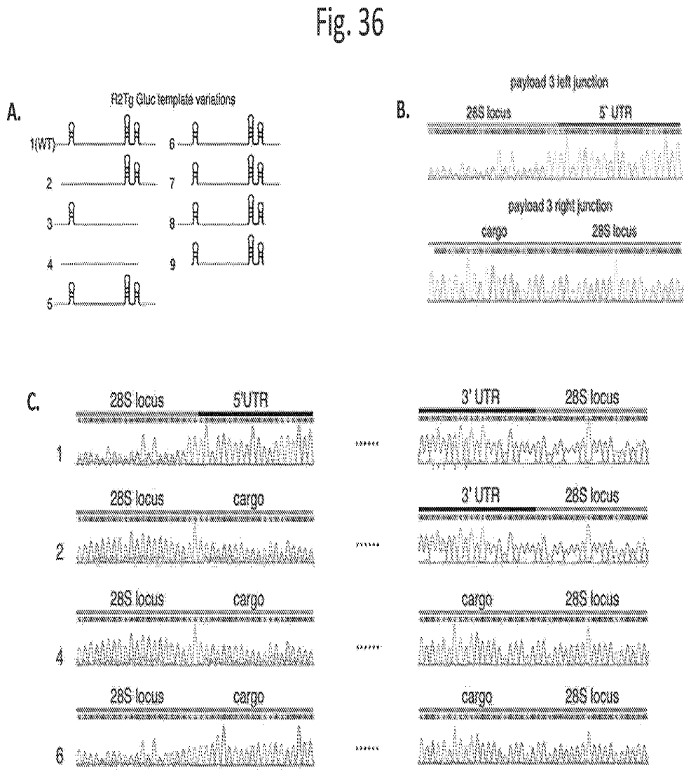

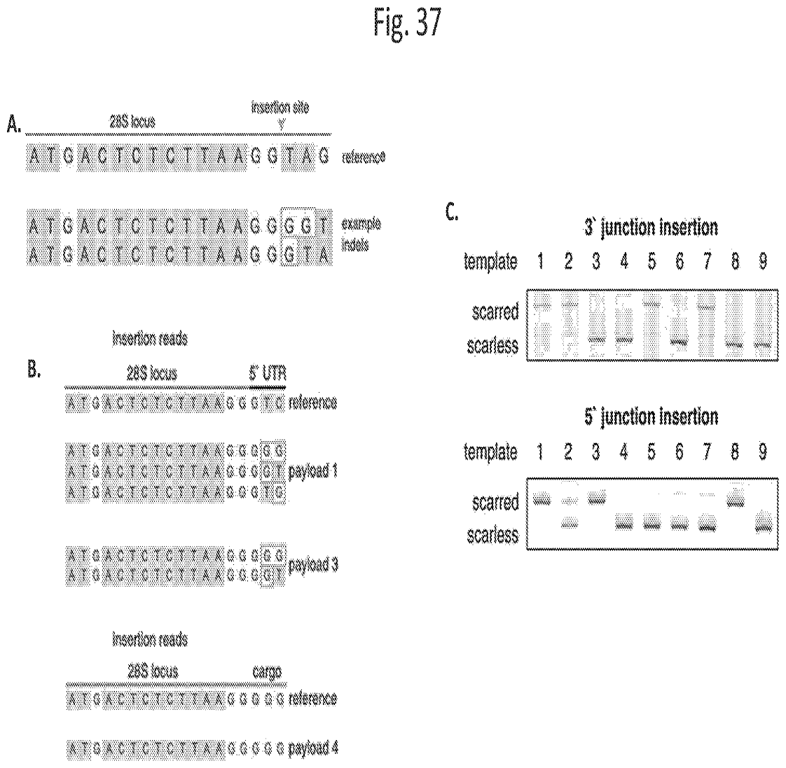

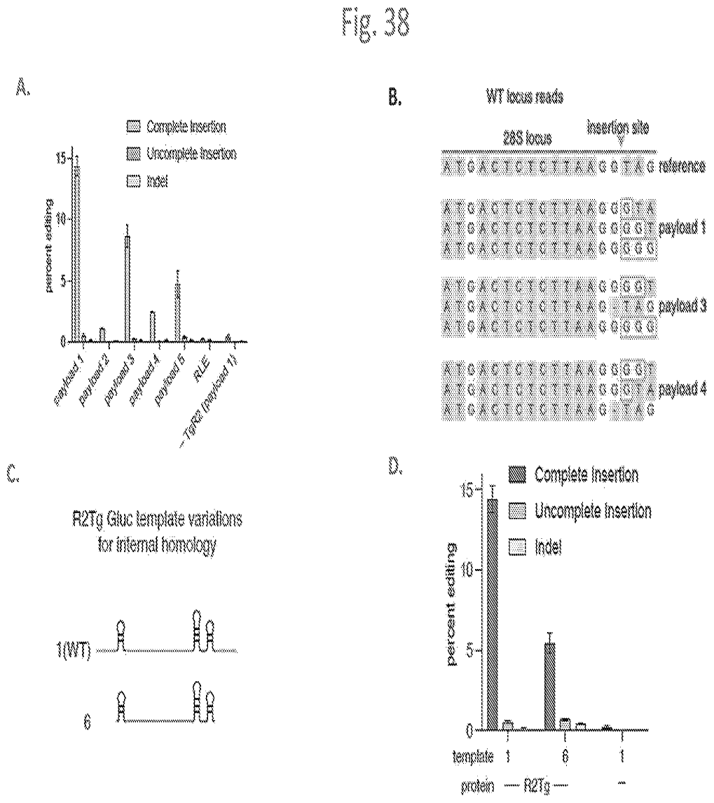

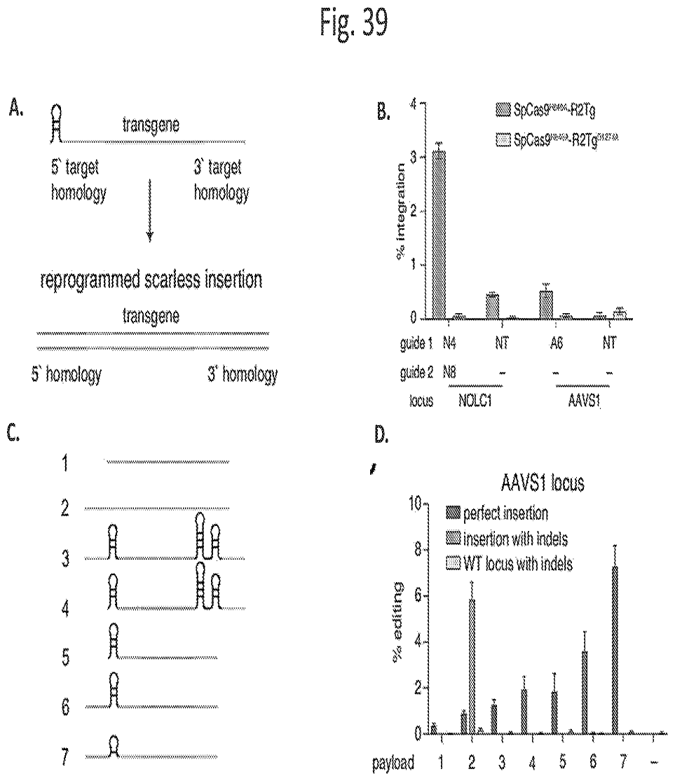

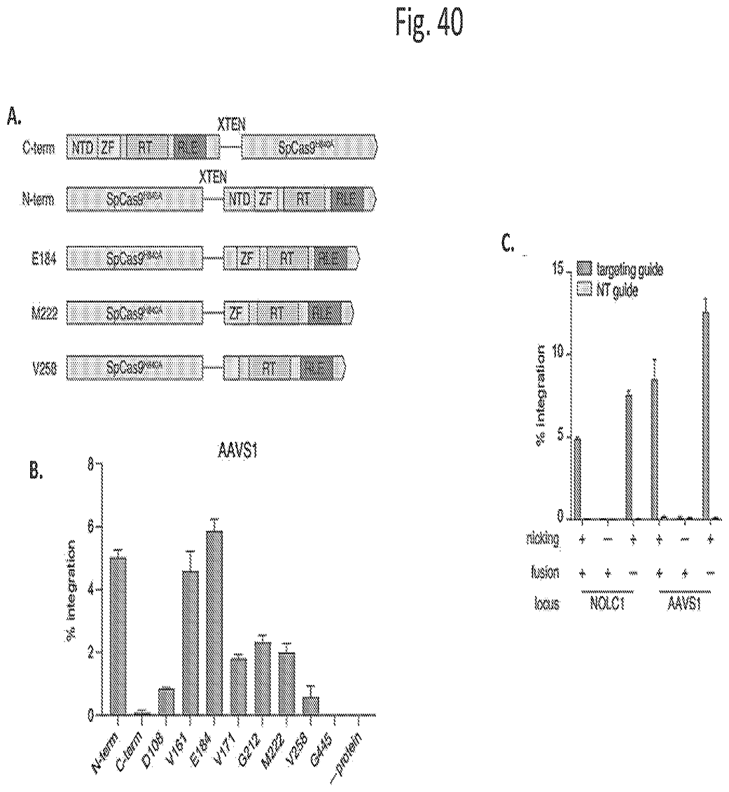

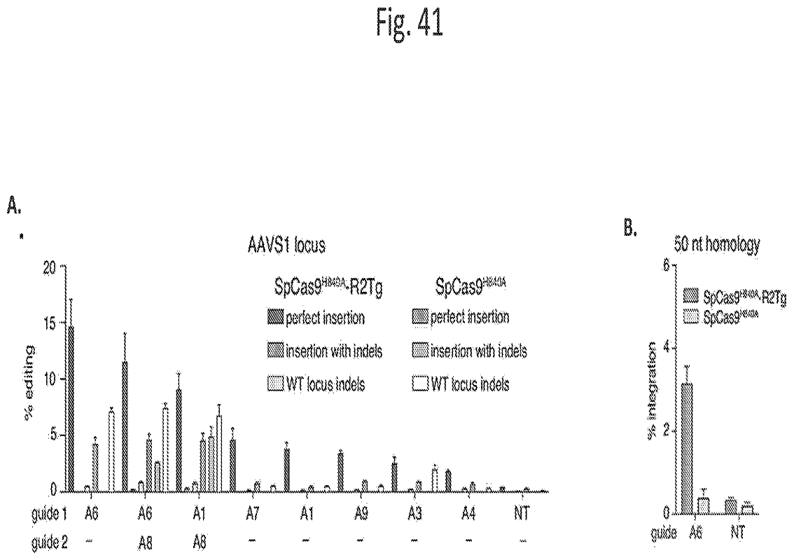

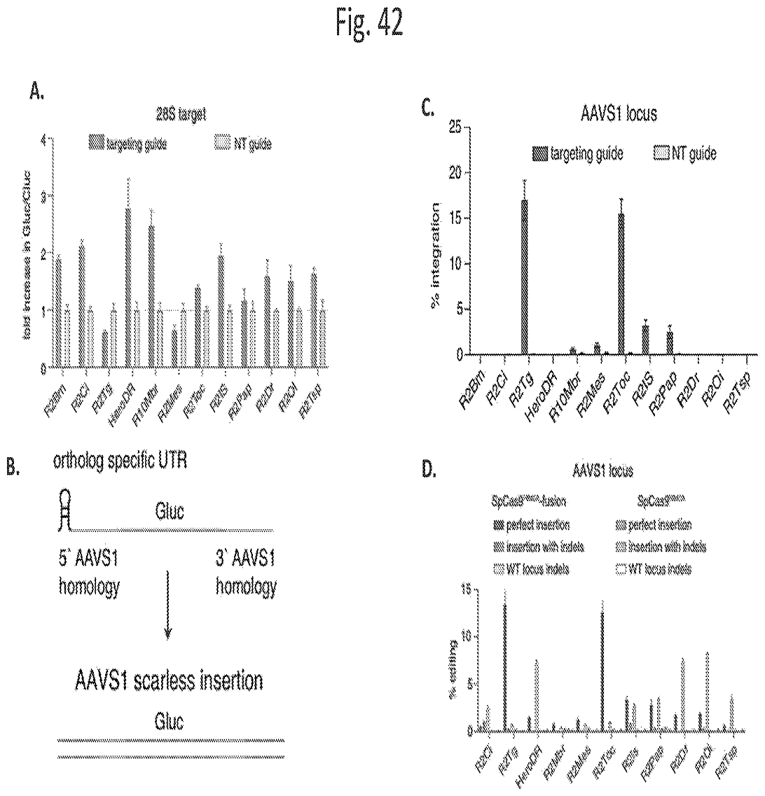

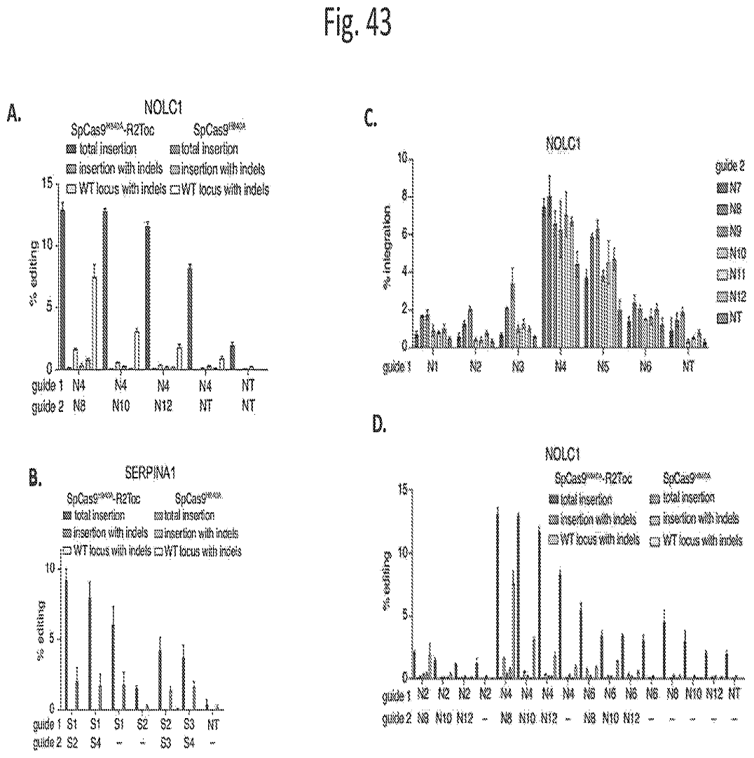

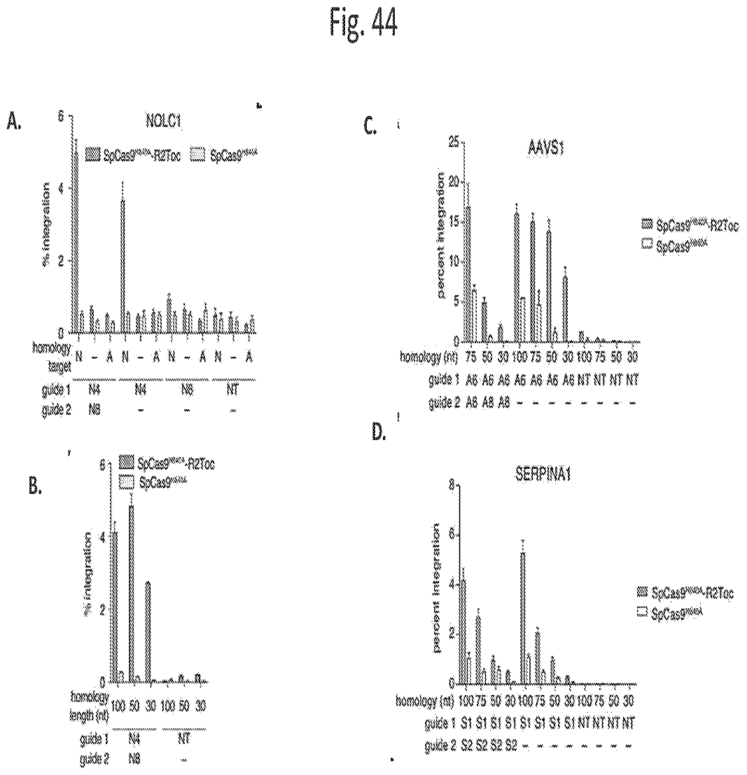

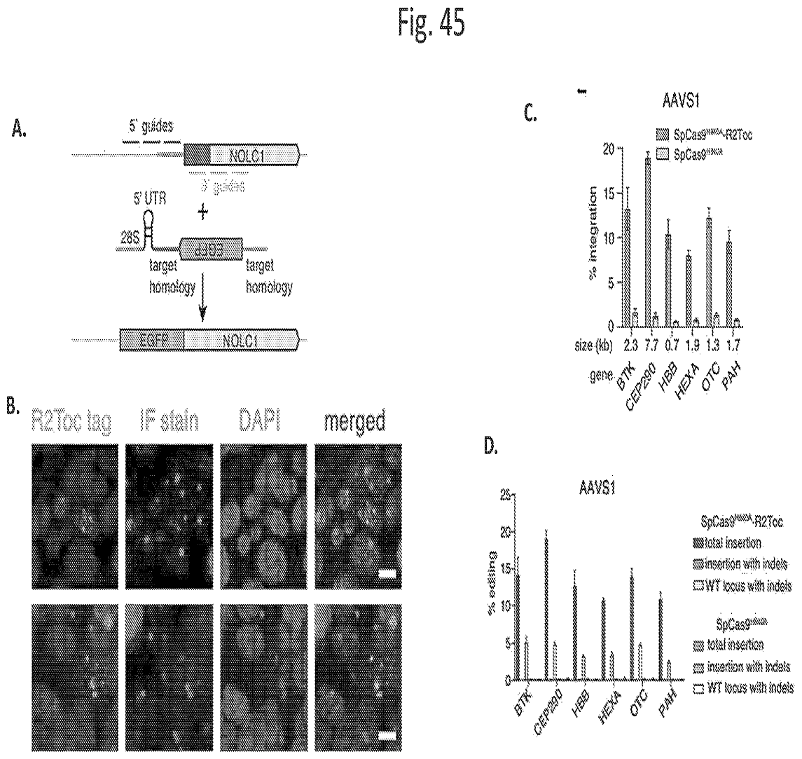

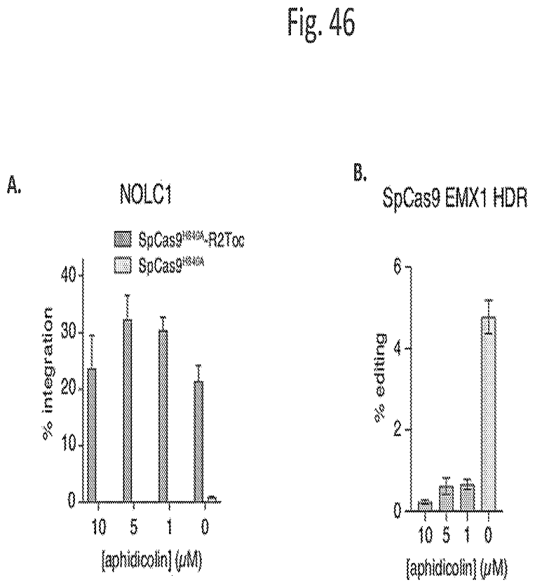

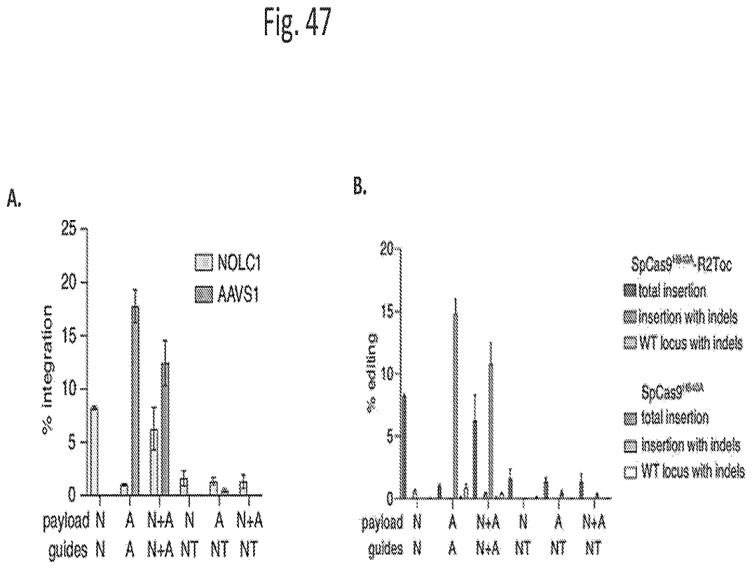

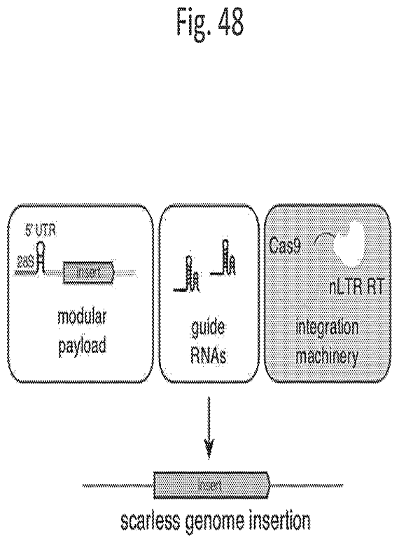

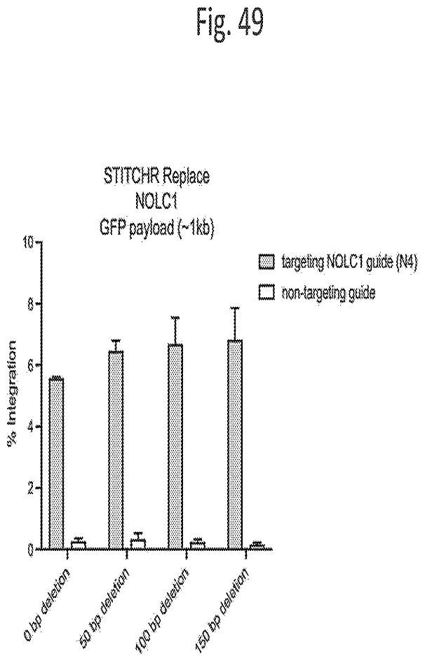

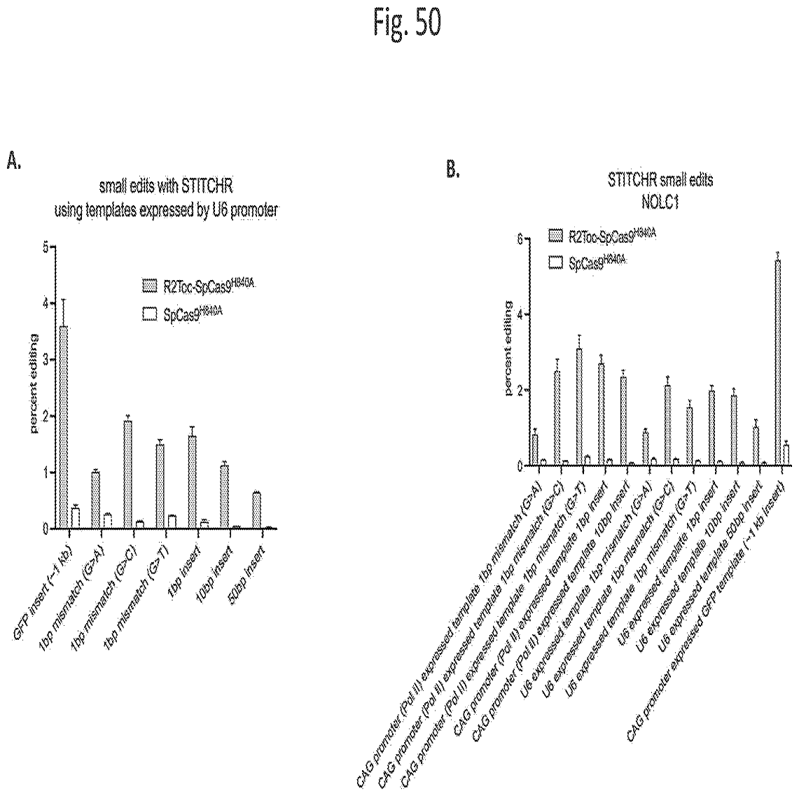

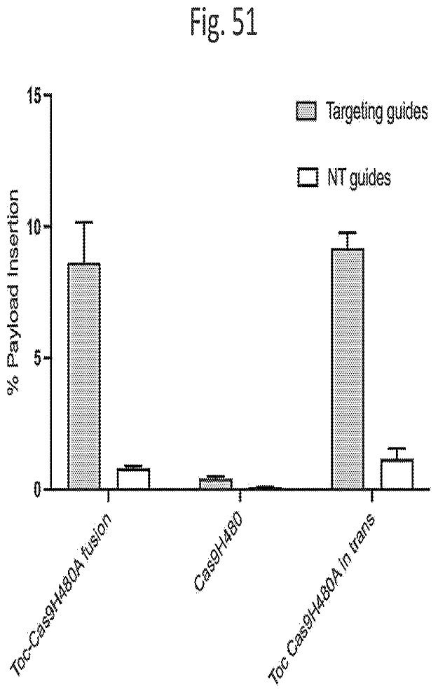

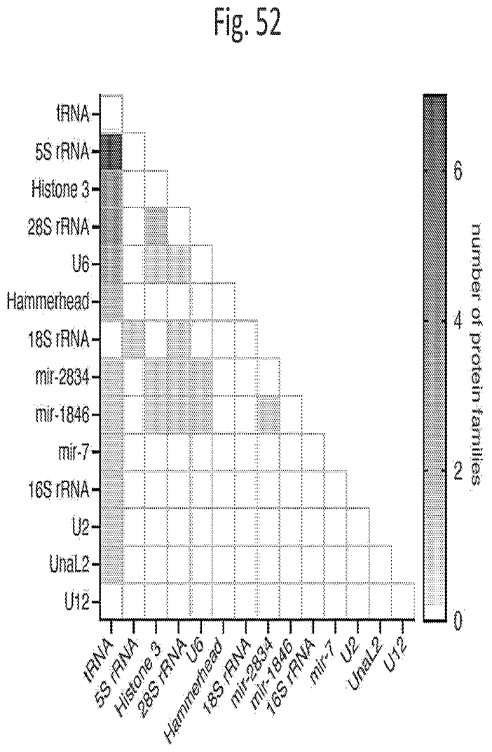

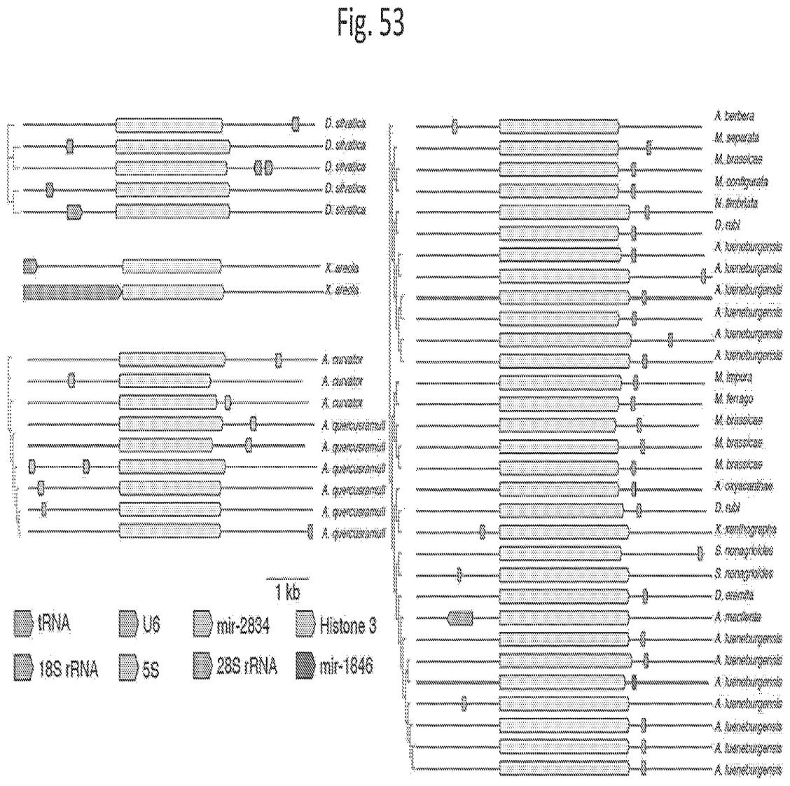

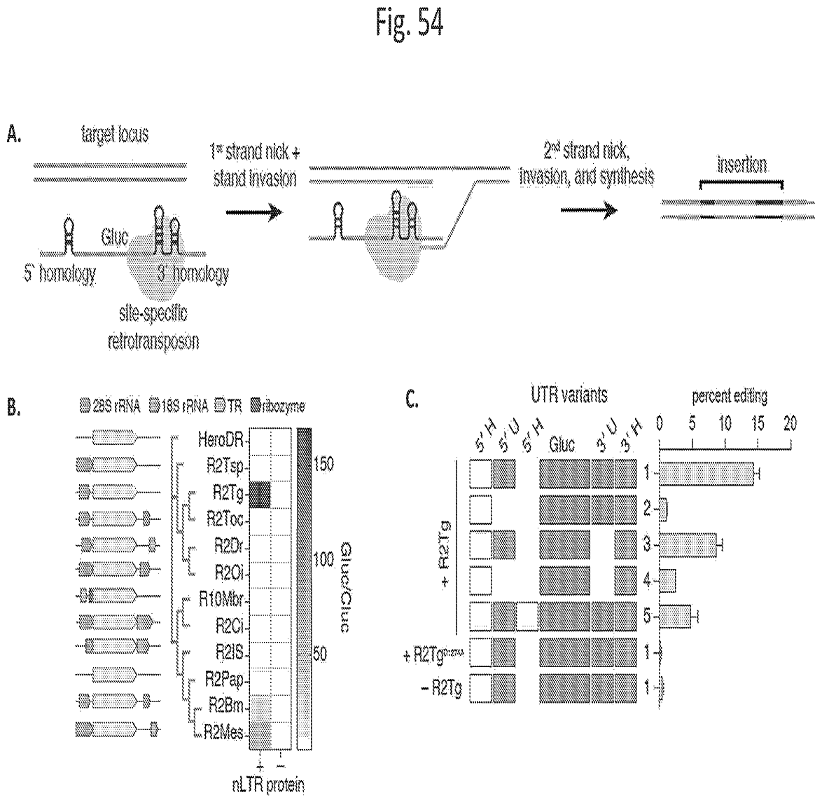

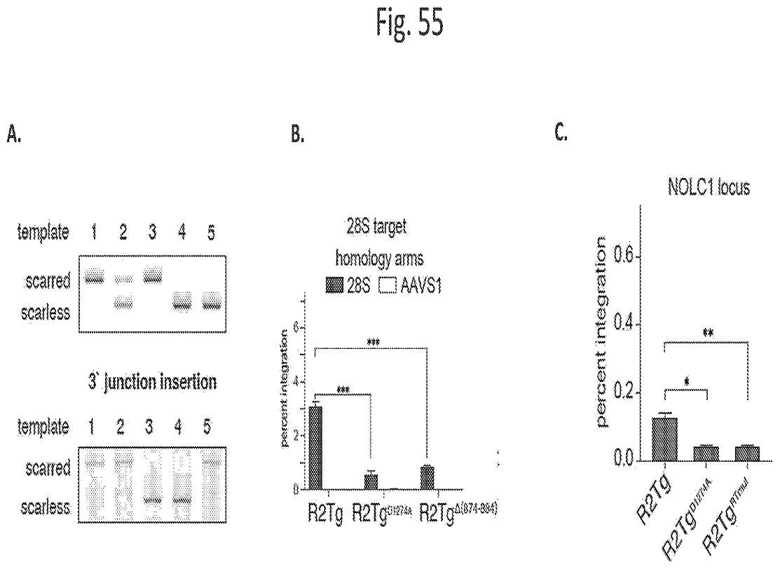

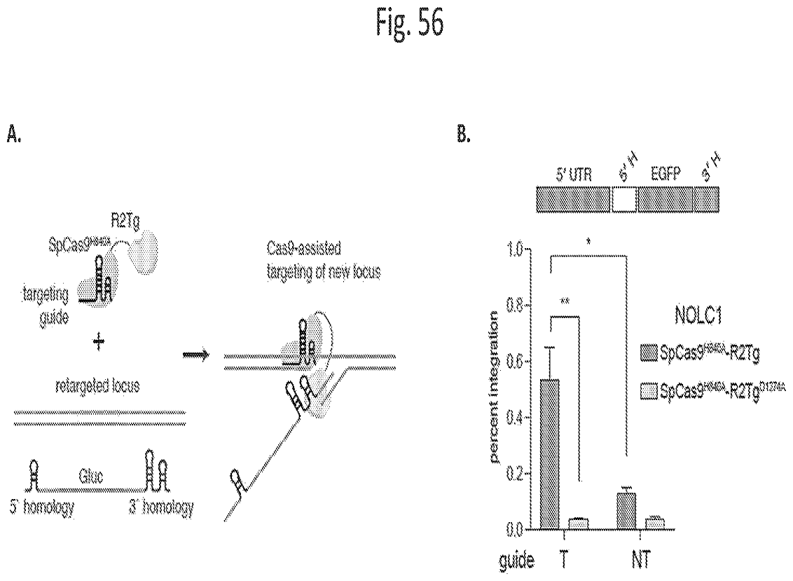

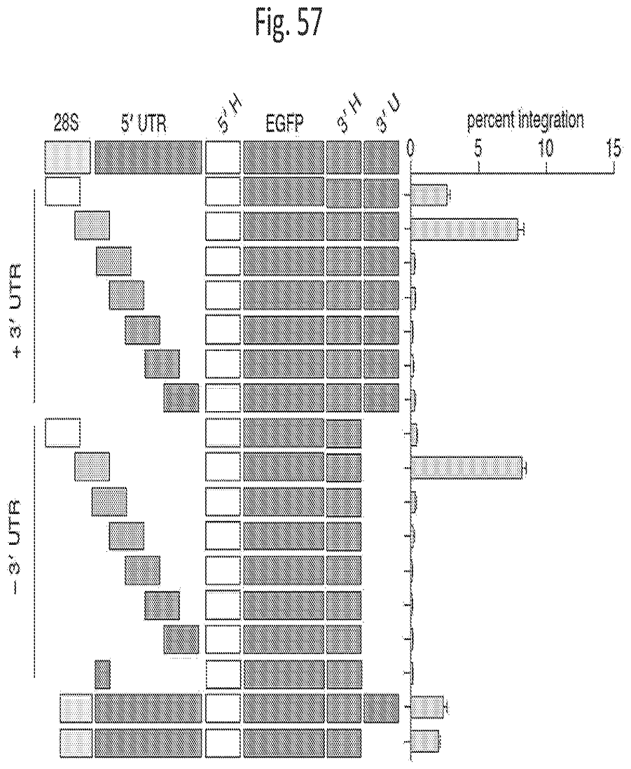

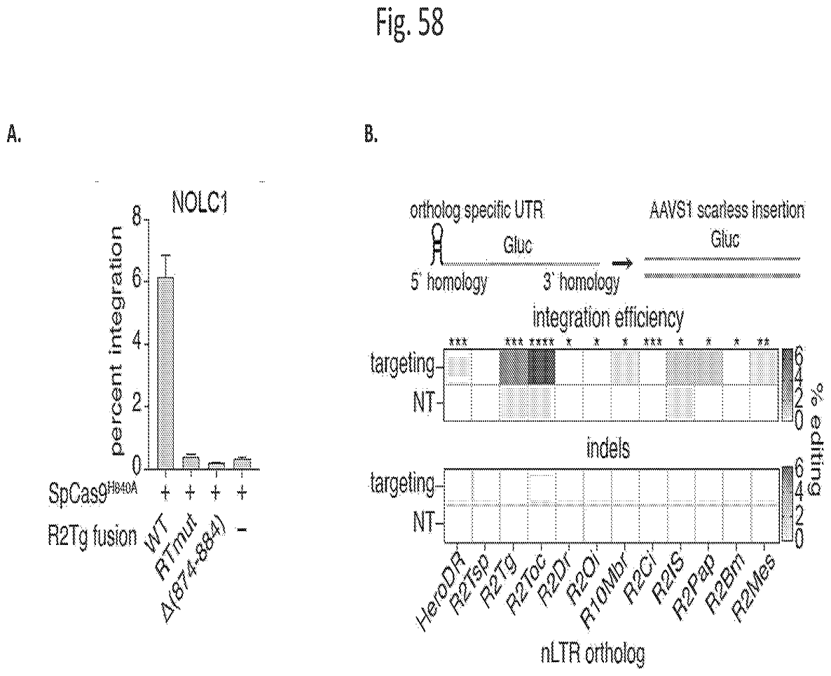

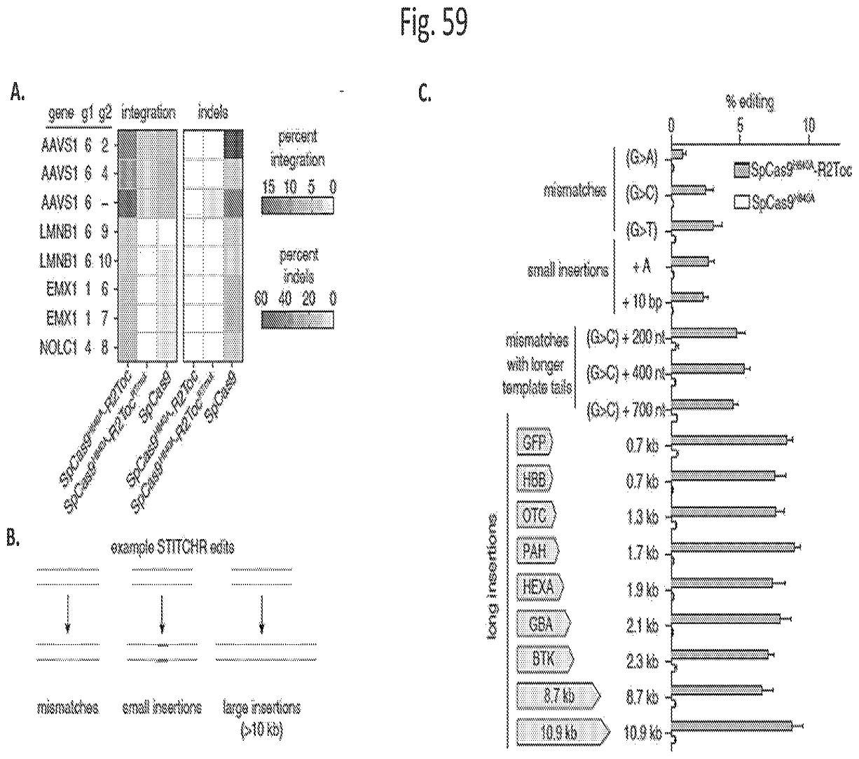

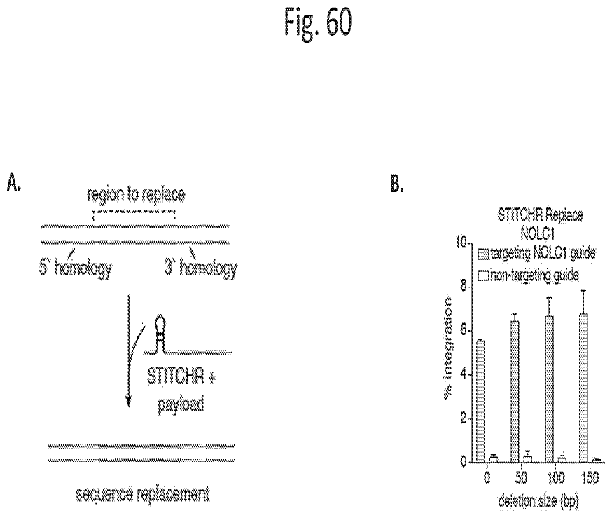

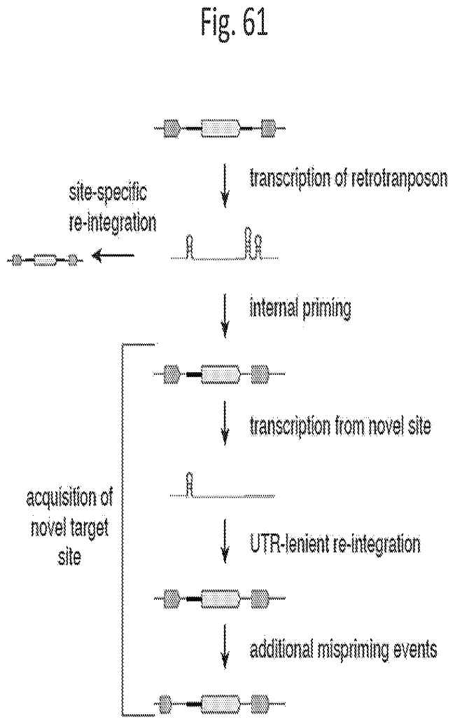

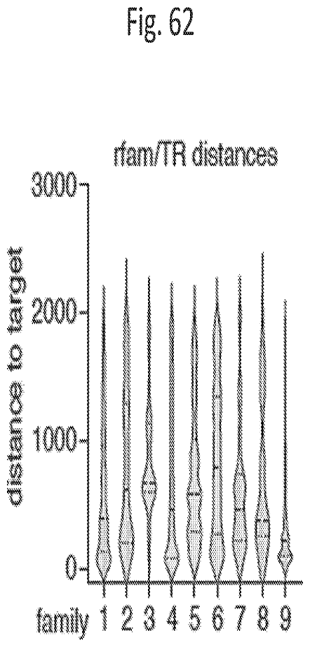

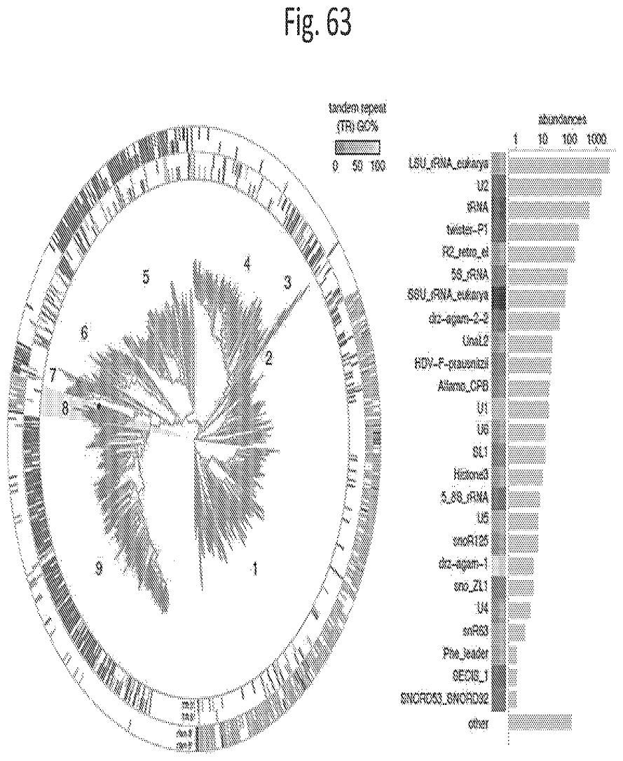

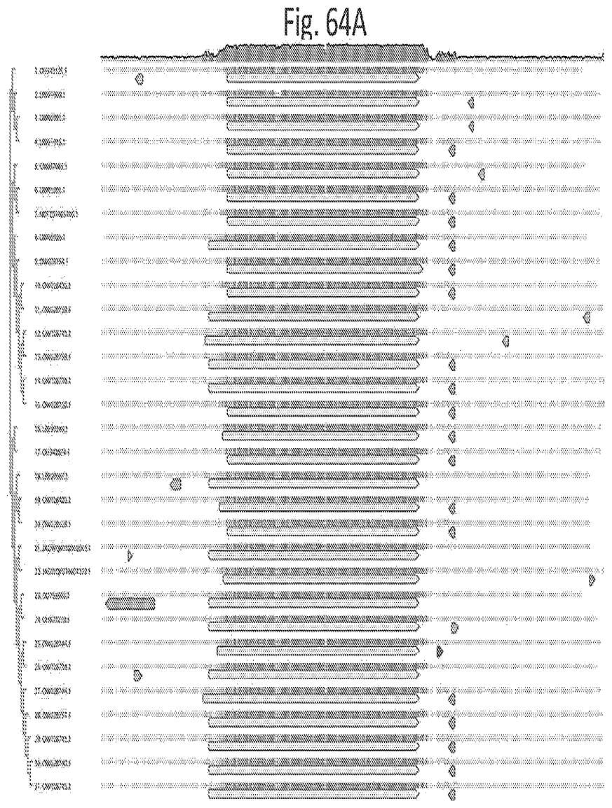

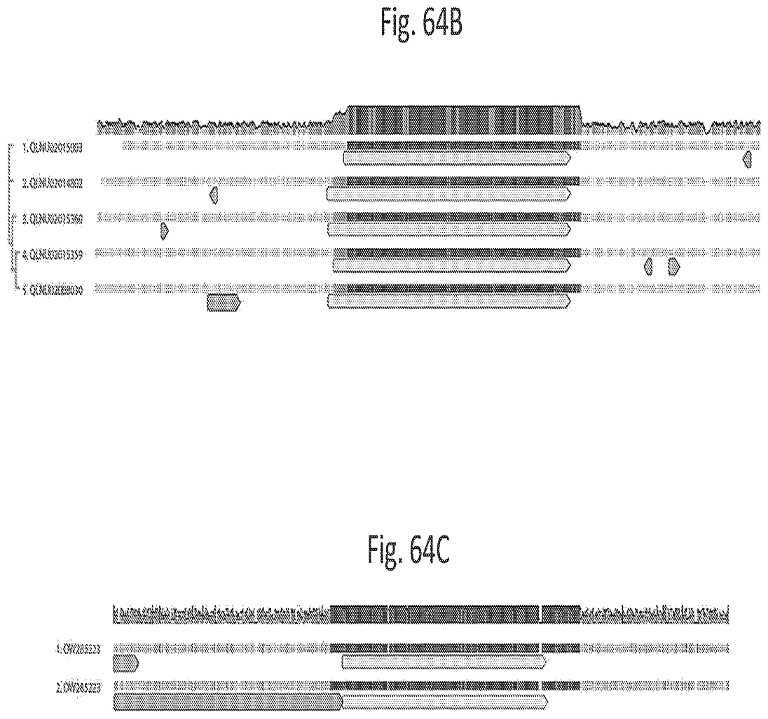

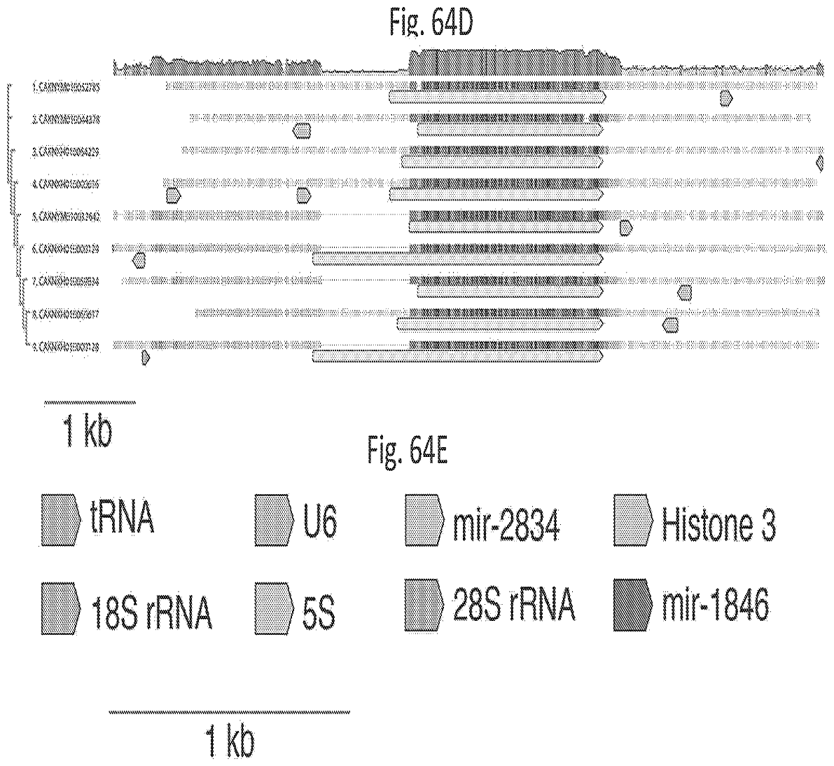

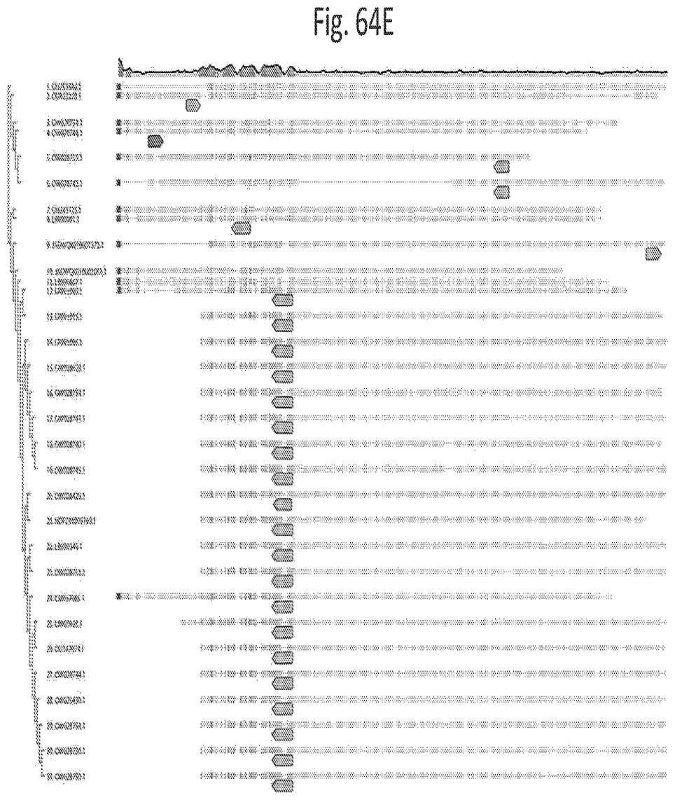

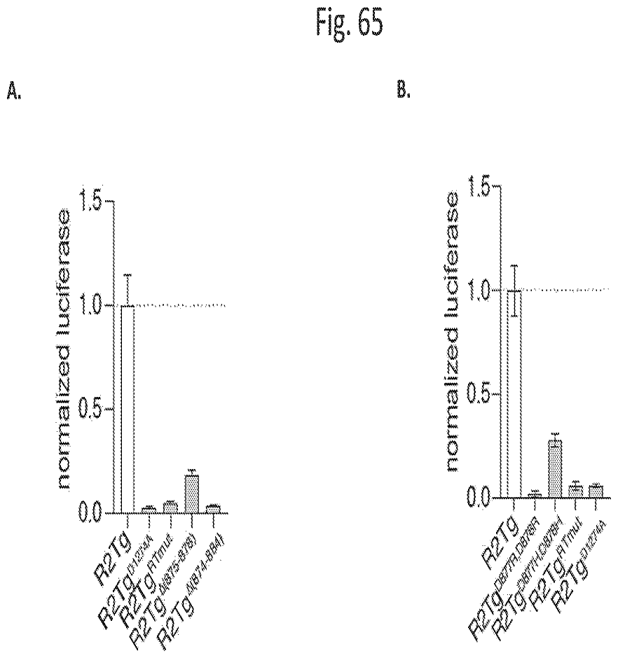

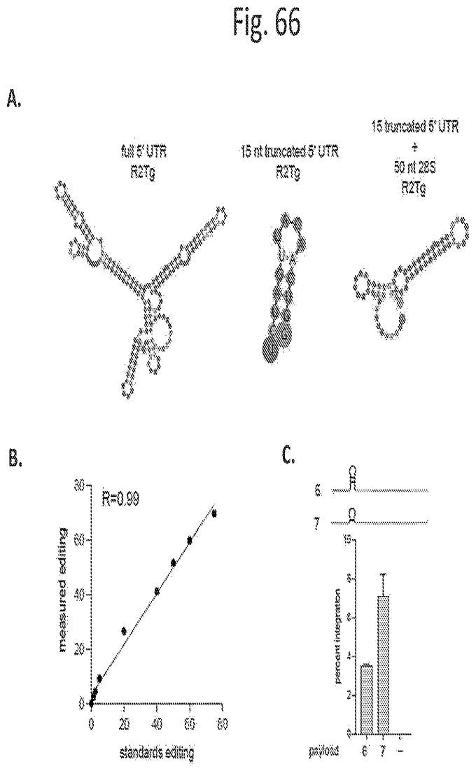

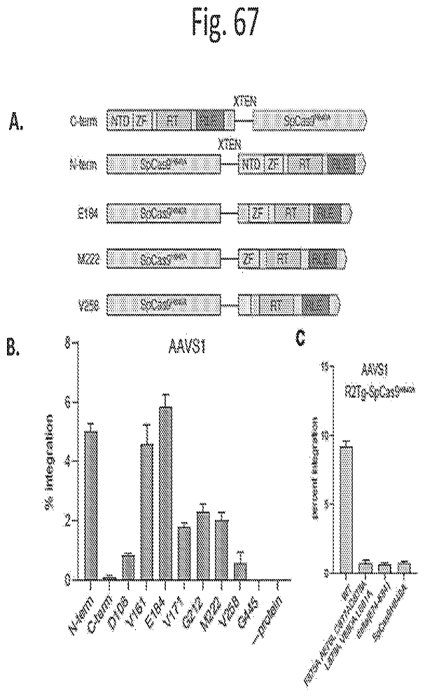

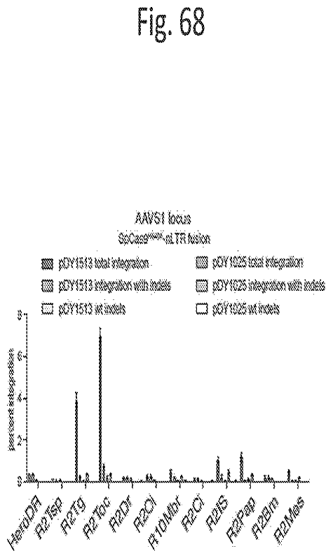

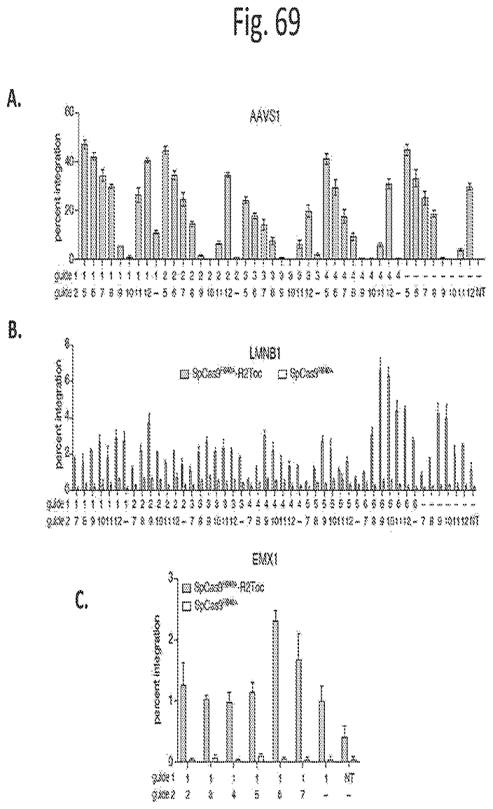

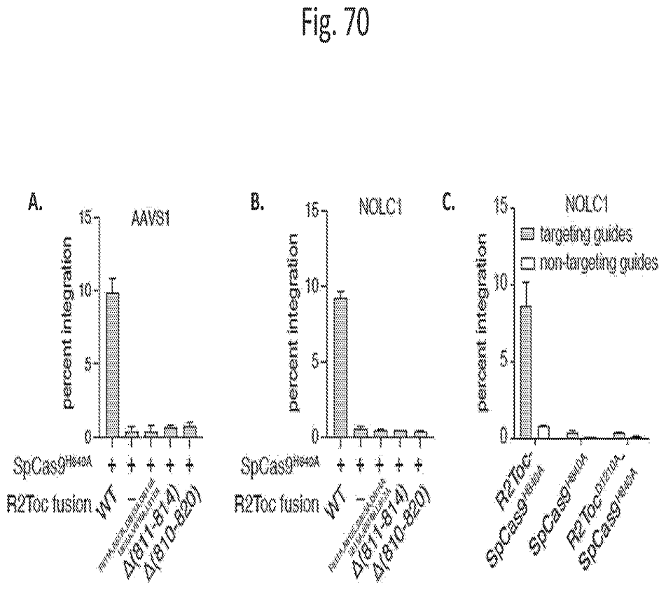

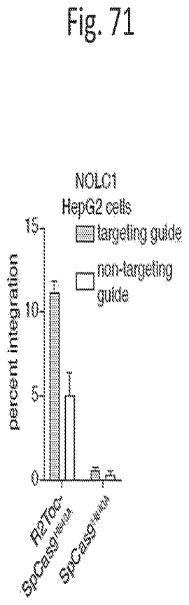

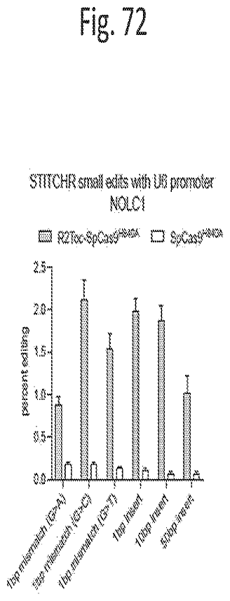

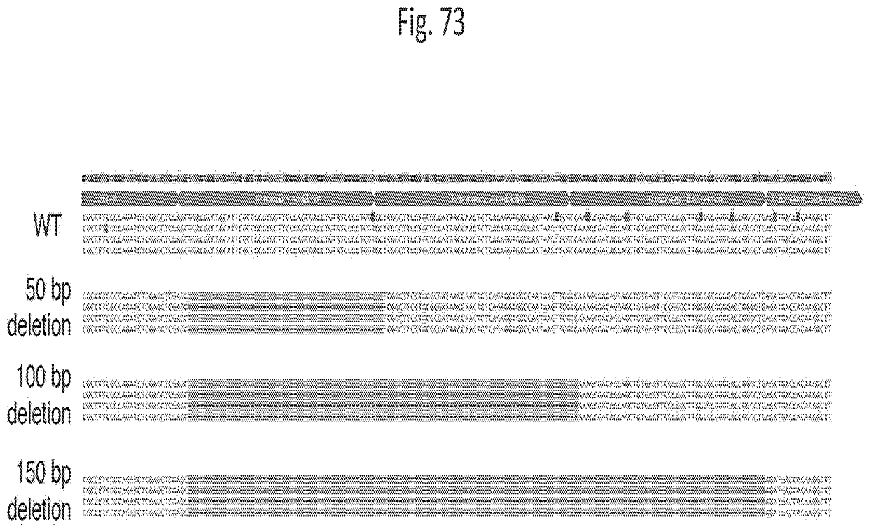

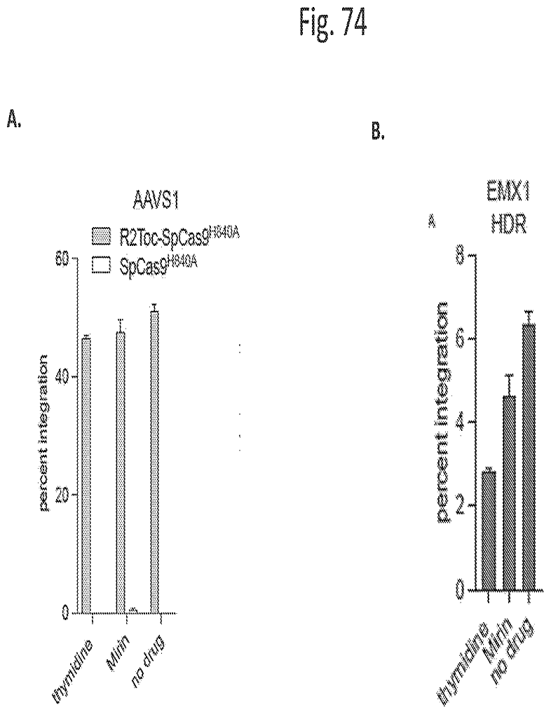

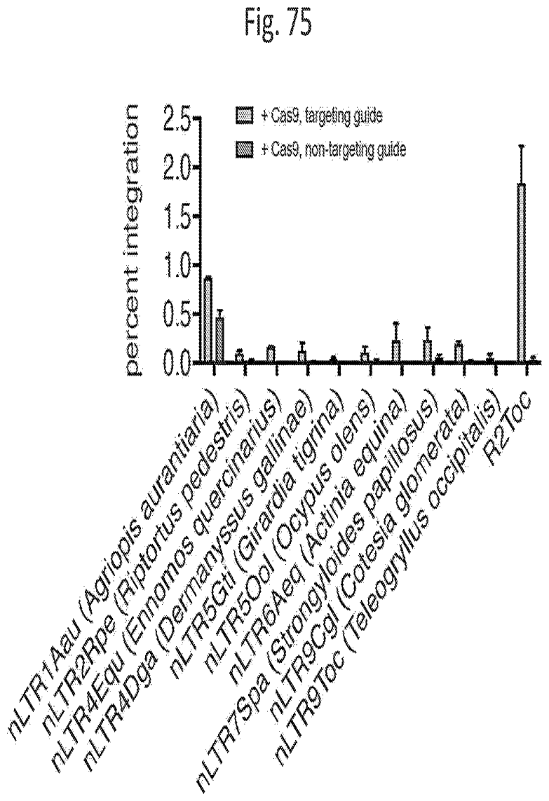

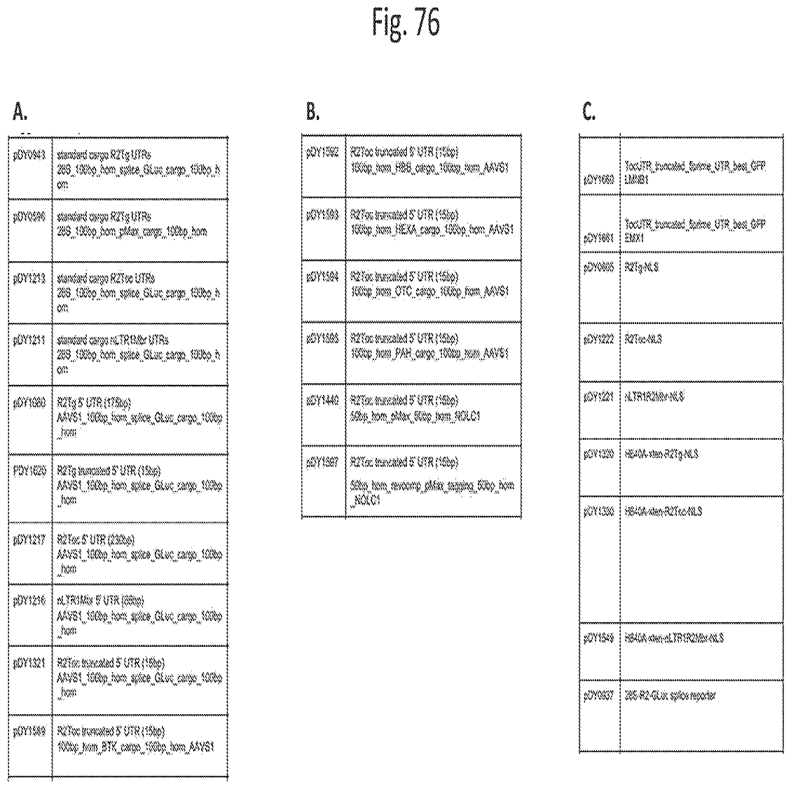

Lastly, the determination of whether these Cas9-R2 fusion proteins were capable of editing human genomes was carried out. HEK293FT cells were stably transfected with a eGFP precursor gene with a 20 bp deletion. As such, the reporter is inactive until the 20 base pairs are inserted into the precursor. FIG. 23A-N exhibit integration and editing efficiency based on the expression of eGFP in these cells. This indicates that the large-scale insertion mechanism of R2 can function in concert with the targeted editing enzyme Cas9 for editing a human genome.

### Example 7. Computational Analysis of Single-ORF Retroelements

The ORFs of 4,464 eukaryotic assemblies (animals and protists) from GenBank for RT, CCHC zinc finger, and RLE domains of known retroelements were also examined. Using a computational pipeline (FIG. 24A) we searched for single protein site-specific non-LTR retrotransposons based on their stereotyped architecture of C-terminal RLE and RT domains as a signature. We identified 8,248 RLE-domain containing representative orthologs (FIG. 24) with diverse RT domains, ZF motifs, and predicted insertion preferences, which clustered into 9 families based on phylogenetic analysis and the presence or absence of flanking rRNA sequences, microsatellite repeats, tRNA genes, or splicing RNAs (Table 1).

We found that families varied in length, with the longer family 3 and 5 ORFs having mean lengths of 1,390 and 1,280 residues, respectively, and family 4 containing shorter ORFs, with a mean length of 966 residues (FIG. 24B, 24C; FIG. 25; Table 1).

The distance to the predicted insertion site, which is indicative of UTR length, also varied substantially, with family 1, 8, and 9 having the least distance between up and downstream annotations and ORF, suggesting shorter UTR lengths (Fig. S1C). While families 1, 3, 5, 6, 7, 8, and 9 associate with previously identified orthologs and subfamilies, families 2 and 4 had no association to known subfamilies (Table 5).

TABLE 5
Summary of characteristics of non-LTR RLE containing retrotransposon families

| | Mean | Mean | | | | | |
|---|---|---|---|---|---|---|---|
| | Length | Length for | Mean | | | | |
| | for full | ORF | distance to | Association | | | 5′ and 3′ |
| | ORF | starting at | target | with known | Integration | RT | homology |
| Family | (bp) | Methioine | (UTR) (bp) | orthologs? | target | architecture | agree? |
| 1 | 1108 | 1055 | 91 | ✓ | 28S, 18S | Non-LTR | x |
| 2 | 1027 | 983 | 778 | x | unknown | Non-LTR | ✓ |
| 3 | 1558 | 1390 | 818 | ✓ | 5S, leader | RT-like | ✓ |
| 4 | 988 | 966 | 666 | x | Tandem | RT-like | ✓ |
| | | | | | repeat | | |
| 5 | 1403 | 1280 | 704 | ✓ | snRNA, | Non-LTR | x |
| | | | | | tRNA | | |
| 6 | 1103 | 1016 | 845 | ✓ | Tandem | Non-LTR | ✓ |
| | | | | | repeat | | |
| 7 | 1185 | 1138 | 584 | ✓ | tRNA | Non-LTR | ✓ |
| 8 | 1262 | 1220 | 351 | ✓ | 28S | Non-ETR | ✓ |
| 9 | 1199 | 1200 | 411 | ✓ | Tandem | Non-LTR | ✓ |
| | | | | | repeat | | |

We next examined the preferred integration sites for these families. Family 1 exhibited a preference for integrating into 28S and 18S rRNA gene sites; family 3 exhibited a preference for integrating into 5S and likely spliced leader sequences; families 4, 6, and 9 exhibited a preference for integrating into tandem repeats and microsatellites, including novel repeat sequences; family 5 exhibited a preference for integrating into snRNA gene loci and some tRNA preferences; family 7 exhibited a preference for integrating into tRNA; and family 8 exhibited a preference for integrating into 28S loci (Table 1). Family 2 has an unknown integration site preference. Accordingly, the zinc finger motifs across these different families are divergent (FIG. 24B, 24C).

Clusters showed two reverse transcriptase (RT) architectures, with families 3 and 4 containing broad RT-like domains, and all other families containing more specific non-LTR retrotransposon RT domains (FIG. 24A, 24B). In contrast to previous efforts profiling RLE-containing non-LTR retrotransposon diversity (Kojima, et al., PLoS One. 11, e0163496 (2016); Eickbush et al., PLoS One. 8, e66441 (2013); Luchetti, et al., PLoS One. 8, e57076 (2013)), our computational expansion covers an order of magnitude more orthologs than the 418 surveyed before (Kojima et al., Genes Genet. Syst. 94, 233-252 (2020); Bao et al., Mob. DNA. 6, 11 (2015).), allowing discovery of families with multiple integration preferences, such as in family 1, where 5S rDNA preferences are interspersed between 28S preferences, or families with discordance between 5′ and 3′ site predictions, such as in family 5.

We next investigated retroelements which had discordant 5′ and 3′ homologies. We found multiple instances of discordant homologies, including in family 1, which has members with 5′ small subunit rRNA preferences and 3′ large subunit rRNA preferences, and family 5 which contains systems with 5′ SL1 splicing leader preferences and 3′ U2 small nuclear RNA (snRNA) target preferences (FIG. 26). Our analysis revealed two broad classes of insertions, which we termed Class 1 and Class 2 based on the nature of surrounding elements (FIG. 26A, 26B). Class 1 insertions include ORFs with a canonical target site on one side and a different target gene on the other side. In contrast, Class 2 insertions involve canonical integration into a target site flanking the ORF retrotransposon, with additional putative insertion targets nearby.

To elucidate divergent target preferences, Rfam annotations were made around all members (FIG. 24A-B, 62, 63) and the results show 188 systems clustering into 39 groups with divergent target preferences (FIG. 52, 53). Many of these divergent protein families show one dominant target site preference with a subset of member systems displaying new preferences such as a change from a 5S to tRNA site preference (FIG. 53).

We also heterologously reconstituted site-specific retrotransposition in human cells to model integration preferences and retargeting in eukaryotic genomes. We synthesized a panel of 12 retrotransposon ORFs from our computational exploration, selecting a sample that included R2 elements with demonstrated activity in mammalian cells (R2Ol) (A. Kuroki-Kami, et al. 2019 Mob. DNA. 10, 23; Su, et al., 2019. RNA. 25, 1432-1438), experimentally characterized retrotransposon groups without proven activity in mammalian cells (R2Bm), previously computationally described retrotransposons (R2Ci, R2Tg, R2Is, R2Pap, R2Dr, R2Tsp, HeroDr) (Kojima et al., 2016 PLoS One. 11, e0163496), and novel retrotransposons (R10Mbr, R2Toc, R2Mes) (Table 6), which all ranged in sequence similarity between 13%-67% (FIG. 27, 64A-E). R10Mbr, which occurs in the genome of Myotis brandtii, appeared to integrate in GTA microsatellites (FIG. 28) and lacked similarity to integration preferences to other families. As such it was designated as a putative R10 family member.

To evaluate the native targeting capacity of these candidates for the 28S loci, we developed a plasmid reporter containing 200 bp of the 28S target with upstream expression of the N-terminus of Gaussia luciferase (Gluc) and delivered a payload containing an exon with 28S homology, predicted UTRs for corresponding orthologs, and a C-terminal Gluc fragment. This system enabled readout of insertion efficiency by luciferase production, and we found that only a limited subset (R2Bm, R2Tg, and R2Mes) had native activity from insertion of this heterologous Gluc cargo in HEK293FT cells (FIG. 29A) as measured by luciferase reporter reconstitution. Interestingly, R2Ol, which has previously been demonstrated to be active for 28S genome insertion (Su, et al., 2019. RNA. 25, 1432-1438), did not have activity in the luciferase reconstitution assay (FIG. 29A). As suggested by the predicted R10Mbr preference for microsatellites, we did not see any production of luciferase in these conditions. To confirm that R10Mbr did not integrate into any putative 28S sites, we evaluated a panel of 28S sites known to have insertion by various site-specific retroelements and found no insertion at the tested 28S targeting sites, including in a 28S region with similarity to sequence flanking the R10Mbr locus (FIG. 29B-C, 65A-B), validating our assignment to a novel R10 family that prefers GTA microsatellites, which do not occur in the human genome (Subramanian et al., 2003. Genome Biol. 4, R13). R10Mbr and R2Toc, which occurs in the Talpa occidentalis genome, are both found in mammalian genomes.

TABLE 6
Exemplary non-LTR retrotransposon orthologs

| | | | SEQ | | SEQ | | SEQ | |
|---|---|---|---|---|---|---|---|---|
| Host | | | ID | Predicted | ID | Predicted | ID | |
| Name | Abvn | Protein | NO: | 5′ UTR | NO: | 3′ UTR | NO: | Accession |
| Daniorerio | Hero | MTTHRAEVTTSGKTQEE | 33479 | TTCAAGCCTG | 33491 | TGATCAACCC | 33501 | LR812084.1 |
| | DR | PGPEATHSAQSLLVSPT | | GCGCAGCCAG | | CGGCTGGGTC | | |
| | | PAAGRSP | | TGACTCCTAG | | ACCTGGGTGA | | |
| | | ATQSCPQVTAAHNSPQS | | GAATAGACTA | | GAGTGTATGA | | |
| | | PQSQQVAVTRSDCVPLA | | GGTGGCAACC | | TGTTGAGAGA | | |
| | | QPRIQWP | | AAGAATAGTT | | CCCGAAACAC | | |
| | | QSSKKAEWLQFDKDVNQ | | TGGTCGACTA | | TCAATGATCC | | |
| | | ILEVTGKGGVDQRLSTM | | CTGGAGAGAC | | CAGGATACAT | | |
| | | TTLIVNI | | AGTTGACGGC | | CACTGATGAT | | |
| | | AAERFGTVTPKPTPSTY | | ACGGAAAGAC | | GTGTCCCAAA | | |
| | | TPSHRVKEIKRLRKELK | | GGCACTTGGG | | TGCATCCATG | | |
| | | LLKRQYK | | ACAGTATGGG | | AGATGTTTCT | | |
| | | AAGEVERAGLEDLRGIL | | TTAGCACCCC | | TGCATAA | | |
| | | RKQLVNLCRAEYHRKRR | | GACCTGTGTC | | | | |
| | | RERARKR | | TTTCGTGAGA | | | | |
| | | AAFLANPFKLTKQLLGQ | | GAGAACCCAA | | | | |
| | | KRTGKLTCSKEAINNHL | | ACAAGCTACG | | | | |
| | | KATYSDP | | GAAAGCCCCA | | | | |
| | | NREQPLGPCGALLTPPE | | CAGAGATATA | | | | |
| | | PTSEFNMKEPCRSEVEE | | CCCCCAGGAG | | | | |
| | | VVRRARS | | ATCCCGAGAG | | | | |
| | | SSAPGPSGVPYKVYKNC | | GGGGGGAGGA | | | | |
| | | PKLLHRLWKALKVIWRR | | TGAGATCTCC | | | | |
| | | GKIAQPW | | AATCGGACGG | | | | |
| | | RYAEGVYIPKEEKSENI | | ATCAAAGGTT | | | | |
| | | DQFRVISLLSVESKIFF | | A | | | | |
| | | SIVAKRL | | | | | | |
| | | SNFLLSNKYIDTSMQKG | | | | | | |
| | | GIPGVPGCLEHTGVVTQ | | | | | | |
| | | LIREARE | | | | | | |
| | | GRGDLAVLWLDLTNAYG | | | | | | |
| | | SIPHKLVEVALEKHHVP | | | | | | |
| | | QKVKDLI | | | | | | |
| | | IDYYSKFSLRVSSGQLT | | | | | | |
| | | SDWHQLEVGIITGCTIS | | | | | | |
| | | VTLFALA | | | | | | |
| | | MNMMVKAAETECRGPLS | | | | | | |
| | | KSGVRQPPIRAFMDDLT | | | | | | |
| | | VTTTSVP | | | | | | |
| | | GARWILQGLERLVAWAR | | | | | | |
| | | MSFKPAKSRSLVLRKGK | | | | | | |
| | | VRDEFRF | | | | | | |
| | | RLGQHQIPSVTERPVKS | | | | | | |
| | | LGKAFNCSLNDRDSIRE | | | | | | |
| | | TSTAMEA | | | | | | |
| | | WLKAVDKSGLPGRFKAW | | | | | | |
| | | VYQHGILPRLLWPLLIY | | | | | | |
| | | EVPMTW | | | | | | |
| | | EGFEQKVSSYLRRWLGL | | | | | | |
| | | PRSLSNIALYGNTNKLK | | | | | | |
| | | LPFGSVR | | | | | | |
| | | EEFIVARTREHLQYSGS | | | | | | |
| | | RDAKVSGAGIVIRTGRK | | | | | | |
| | | WRAAEAV | | | | | | |
| | | EQAETRLKHKAILGAVA | | | | | | |
| | | QGRAGLGSLAATRYDSA | | | | | | |
| | | SGRERQR | | | | | | |
| | | LVQEEVRASVEEERTSR | | | | | | |
| | | AVAMRQQGAWMKWEQAM | | | | | | |
| | | ERNVTWK | | | | | | |
| | | DIWTWNPLRIRFLIQGV | | | | | | |
| | | YDVLPSPSNLYIWGRVE | | | | | | |
| | | TPACPLC | | | | | | |
| | | SKPGTLEHILSSCSKAL | | | | | | |
| | | GEGRYRWRHDQVLKSIA | | | | | | |
| | | EAISKGI | | | | | | |
| | | KDSRYRQATAKVIQFIK | | | | | | |
| | | EGQRPERTAKNCSAGLL | | | | | | |
| | | STARDWV | | | | | | |
| | | MTVDLERQLKIPPHITQ | | | | | | |
| | | STLRPDIILVSEATKQL | | | | | | |
| | | ILLELTV | | | | | | |
| | | PWEERMEEAQERKRGKY | | | | | | |
| | | QELVEQCRANGWRTRCM | | | | | | |
| | | PVEVGSR | | | | | | |
| | | GFASYTLSKAYGTLGIT | | | | | | |
| | | GTNRRRALSNNVEAAEK | | | | | | |
| | | ASRWLWL | | | | | | |
| | | KRGEQWGQ | | | | | | |
| Myotis | R10Mbr | MGLTTPPGFIVLVTIET | 33480 | TGCCGACTGG | 33492 | GGATCTGCAA | 33502 | NW_005337413.1 |
| brand | | ENDISPGVPTPAYTSTQ | | GCAGCTTTGG | | TTTACCATTG | | |
| | | EGRAEL | | AGAAATCCTA | | GTTCAACTCC | | |
| | | ACGSCGKICKSKAGLVS | | AGCTGGCTAA | | GTGTGACGGG | | |
| | | HRKVHVQGNANSQSGCP | | GAGTTCAGCA | | CACATCGTGC | | |
| | | FTDVDR | | ACTCCTG | | CCCGATGTGT | | |
| | | TCRICDRQFSSKSGLTQ | | | | GAGCCTGGAC | | |
| | | HKRHRHPEARNQEKLSC | | | | TGTGGTAGCA | | |
| | | MKTAGS | | | | CTTCGGTGCA | | |
| | | HWTEQESTALLRIATKL | | | | CTTGAAGAGC | | |
| | | APTCSNLRSLYCRLEHD | | | | AATGTCAGTT | | |
| | | FPGRSA | | | | GTCCGTGTAT | | |
| | | CSIKTRLRTLNWKPTRV | | | | TCTTCATTCT | | |
| | | TLPDDVCVASQESTNND | | | | TCGAAC | | |
| | | TQRIEW | | | | | | |
| | | ASKTVDVAIRQLKDSPQ | | | | | | |
| | | ESLRSADLLAMAESFQR | | | | | | |
| | | GAIDSQ | | | | | | |
| | | QLLSLLEMHAVSTFPHR | | | | | | |
| | | WRMNTKGHARRANATYK | | | | | | |
| | | NRKQIR | | | | | | |
| | | RANYASLQALYHQRRKD | | | | | | |
| | | AATAVFSGTWKDAHLST | | | | | | |
| | | RGLPDN | | | | | | |
| | | SDKYWQDILSAPSHCDN | | | | | | |
| | | RPCRSVTPIDWSLIEPI | | | | | | |
| | | HHEEVT | | | | | | |
| | | SAVKQMGNTAPGLDKIR | | | | | | |
| | | PAELKHYSSKALAGYFN | | | | | | |
| | | LLLLSE | | | | | | |
| | | GCPEHLCLSRITLVPKV | | | | | | |
| | | PNPSCPSELRPIAVSSS | | | | | | |
| | | IIRCFH | | | | | | |
| | | KIIADRWNSRLSLPSLQ | | | | | | |
| | | FAFLKRDGCLEATSTLH | | | | | | |
| | | AILRHS | | | | | | |
| | | CSTGSGLSVAFIDVAKA | | | | | | |
| | | FDSVSHETIIRSAKAFG | | | | | | |
| | | APPPLT | | | | | | |
| | | QYLTTSYERAAAAISTS | | | | | | |
| | | TVKCHRGVRQGDPLSPL | | | | | | |
| | | LFIMAM | | | | | | |
| | | DEVLSSSMPQLGYQFHD | | | | | | |
| | | TLVDGFAYADDLIIMAE | | | | | | |
| | | NLPRLQ | | | | | | |
| | | EKLDAASVALGFAGMKI | | | | | | |
| | | NAKKTKLLDIRGARKPY | | | | | | |
| | | VTATCE | | | | | | |
| | | TPVSFQNEEIKPLSSTE | | | | | | |
| | | TLTYLGIPFTSKGKASI | | | | | | |
| | | NHRRQL | | | | | | |
| | | QEVLSQIRKAPLKPQQR | | | | | | |
| | | LELTREHLIPKYTHTLV | | | | | | |
| | | LGNAHR | | | | | | |
| | | NTLKRMDNAIRQSLRDW | | | | | | |
| | | LRLPPDTPTAYFHTACS | | | | | | |
| | | LGGLGV | | | | | | |
| | | PCLSTTIPLYKKTRMEK | | | | | | |
| | | LLTATCPVLRNVVNSGS | | | | | | |
| | | FKPIIK | | | | | | |
| | | ELSIPIRVHGTIVTDKE | | | | | | |
| | | GAREAWHEHLLSSVDGR | | | | | | |
| | | GLRDVA | | | | | | |
| | | KSPLSNAWLIRPERIFP | | | | | | |
| | | RIFLRAVHLRCNLLRTK | | | | | | |
| | | VRSARG | | | | | | |
| | | GRGDQSVLCRGNCGQPE | | | | | | |
| | | SLAHILQSCWVTHDARC | | | | | | |
| | | ARHNRV | | | | | | |
| | | AKELARRLRKLGYSVFE | | | | | | |
| | | ELRVPTSHSFIKPDLIV | | | | | | |
| | | VQDTSA | | | | | | |
| | | FVLDVSIVGDGRMQSAW | | | | | | |
| | | SEKVEKYSTEAHTAAIS | | | | | | |
| | | SMLSSI | | | | | | |
| | | GKPVEHVFHEPVIFSFR | | | | | | |
| | | GVCYSRSVKSIIRLGLP | | | | | | |
| | | RYSISD | | | | | | |
| | | LCLLTIIGSLRTYDTFM | | | | | | |
| | | RGTWK | | | | | | |
| Phlebotomus | R2PaP | METNRENIYSRDEAGVN | 33481 | AACTATGACG | 33493 | CCTACGGCGA | 33503 | AJVK01060896.1 |
| papatasi | | SLGSRPQMRPRSQTMER | | TATGTTATAG | | AGTTTGCGGC | | |
| | | SIVEAG | | GGAGTTTTAT | | GTTACCATTC | | |
| | | CDQNEFGCDLCDRRFRT | | TAGTTAAGGT | | TGGGTGCTAA | | |
| | | TRGLGQHFRHSHPREHN | | TGGGTGCGTG | | GACTGAACAA | | |
| | | DRLNTD | | GAGTCGGATC | | GGCATTGATG | | |
| | | RIKARWSPEEEYLLALE | | GTTGAAGTCT | | GGTTCCATTG | | |
| | | EVRATSRGIRFLNQHLA | | TCATTGACCT | | TCTAAGGTTG | | |
| | | EAFPNR | | AAATGTATCG | | CTTTTATATT | | |
| | | TIEAIKCHRRQRTYKEL | | TTGACCATCG | | GAGGTGTGCC | | |
| | | VANLLVRASSARESQTS | | TAGCCCTTCA | | GTCGAGCACC | | |
| | | YAGRLG | | AGTGTCCATC | | TGGTAGCATT | | |
| | | ETSSSVSAELVVEVNRL | | TGACGCCCCC | | CATTCTTATG | | |
| | | IDYLAIHPVRKYFSDEL | | TCATGGCGAC | | GGCGAAAGAA | | |
| | | VAAAHA | | CGCTGGGGAT | | TGATAAATAT | | |
| | | AIIGDVECDELILSWLQ | | TGCTTTCGAG | | GATGATCGCG | | |
| | | KAFRIRHGLRPSTSTAA | | CATGAGCTAA | | AGATCATGAT | | |
| | | GNPGSY | | GAGCAGTGGA | | CCACTTTCTT | | |
| | | RSGSDRPLSNRKRRRQD | | TGCGGGGGTG | | GGCGTAATGT | | |
| | | YARVQRLWNKSVKKAAR | | GTACAGGCGT | | GGAAAGTCTA | | |
| | | GILEGS | | ATCACCCTTA | | GCATGGTAAT | | |
| | | DEANSGESVHPTPERML | | AAAAGAAAGT | | GGGGTAAAGT | | |
| | | RYWSDIFKQEGPIIPDR | | CGATC | | TGGGTCTCTG | | |
| | | TNQSPR | | | | ATCCAGGCTA | | |
| | | NEELKDMWEPITIDEVK | | | | TACCTATGAT | | |
| | | LARLDPGSAAGIDRISV | | | | GAGAAACCCT | | |
| | | QQFQRC | | | | AGTTCACTTG | | |
| | | PVHVRVLLFNVLLLVGH | | | | CTCAATTCTA | | |
| | | LPGRMSCARTVFLPKVE | | | | TTTGTCGTAA | | |
| | | GSSDPK | | | | GACTTATGGA | | |
| | | DYRPISITSVITRQFHK | | | | AATAAGTGAC | | |
| | | ILAARLTSMHAWDERQA | | | | AAAACGATCT | | |
| | | GFLPVD | | | | AGACTATTTC | | |
| | | GCGENLAILNELIRFSR | | | | TGAGGGTA | | |
| | | VNRRELHLASLDISKAF | | | | | | |
| | | DMVPRQ | | | | | | |
| | | AIINSVAQLGAPQNLVE | | | | | | |
| | | YLKGLYANNQTTLEYGG | | | | | | |
| | | SELYCR | | | | | | |
| | | VKRGVRQGDPLSPLLFN | | | | | | |
| | | LVMESALVRLDKKLSFS | | | | | | |
| | | LYGVSV | | | | | | |
| | | NGLAYADDVILVASTSG | | | | | | |
| | | GLQKNTESFLGALREIG | | | | | | |
| | | LDLNLA | | | | | | |
| | | KCKSLSLKPSGRDKRCK | | | | | | |
| | | VLSESQLSIGGTSVPQV | | | | | | |
| | | DLVGFW | | | | | | |
| | | RYLGIWFSGPRVVSPEQ | | | | | | |
| | | LSMGVYLERISKAPLKP | | | | | | |
| | | QQRIRI | | | | | | |
| | | LVDYLLPKYTHGSVLGR | | | | | | |
| | | YTRKTYKAMDAQIRSYV | | | | | | |
| | | RKWLHL | | | | | | |
| | | PLDTTLGYFYAPVMSGG | | | | | | |
| | | LGIPNFEMTVPLMKVER | | | | | | |
| | | NRKLLS | | | | | | |
| | | SARGTIRAVMHGSPLIR | | | | | | |
| | | DTERTASWLLTRLPALD | | | | | | |
| | | IECYKG | | | | | | |
| | | YWIKSLYESSDGRDNRA | | | | | | |
| | | INGVRGSIGWSRKFSNK | | | | | | |
| | | LTGRDF | | | | | | |
| | | VHFHQIRINALDSKART | | | | | | |
| | | LRGRGVDVRCRAGCLDR | | | | | | |
| | | ETPYHI | | | | | | |
| | | VQRCFRSHGGRVLRHDN | | | | | | |
| | | SVQLLCSEMTRKGYNVA | | | | | | |
| | | VERQLQ | | | | | | |
| | | TVEGMRKPDLIAVKDGR | | | | | | |
| | | AAVIDMQVVSGGSMESS | | | | | | |
| | | HREKVE | | | | | | |
| | | KYQRIPGYTELVKEAFG | | | | | | |
| | | VASVEYRAATISWRGIW | | | | | | |
| | | FKPSYD | | | | | | |
| | | SLTRLGVGERCLGSICC | | | | | | |
| | | QVMRGSYLNFVRFKQST | | | | | | |
| | | QMVWSAV | | | | | | |
| Bombymori | R2Bm | MMASTALSLMGRCNPDG | 33482 | GGGCGATACG | 33494 | GCCTTGCACA | 33504 | AB076841.1 |
| | | CTRGKHVTAAPMDGPRG | | CATAATTTTA | | GTAGTCCAGC | | |
| | | PSSLAG | | ATTTCCCGAT | | GGTAAGGGTG | | |
| | | TFGWGLAIPAGEPCGRV | | TGAAATCCAG | | TAGATCAGGC | | |
| | | CSPATVGFFPVAKKSNK | | TCGTCTTAAT | | CCGTCTGTTT | | |
| | | ENRPEASG | | CTGGTGACCA | | CTTCCCCGGA | | |
| | | LPLESERTGDNPTVRGS | | GTGGCGCGGT | | GCTCGCTCCC | | |
| | | AGADPVGQDAPGWTCQF | | CACCAGTATA | | TTGGCTTCCC | | |
| | | CERTFST | | GTGCACAGGA | | TTATATTTAA | | |
| | | NRGLGVHKRRAHPVETN | | CGTGAATGGC | | CATCAGAAAC | | |
| | | TDAAPMMVKRRWHGEEI | | TCCGAGGCTG | | AGACATTAAA | | |
| | | DLLARTE | | GCGGAGTCAc | | CATCTACTGA | | |
| | | ARLLAERGQCSGGDLFG | | tcactataag | | TCCAATTTCG | | |
| | | ALPGFGRTLEAIKGQRR | | tgtgagagac | | CCGGCGTACG | | |
| | | REPYRALV | | gatgtcctgt | | GCCACGATCG | | |
| | | QAHLARFGSQPGPSSGG | | gccaagtata | | GGAGGGTGGG | | |
| | | CSAEPDFRRASGAEEAV | | cgtccaaccc | | AATCTCGGGG | | |
| | | EERCAED | | taacgggtta | | ATCTTCCGAT | | |
| | | AAAYDPSAVGQMSPDAA | | agtgaaatta | | CCTAATCCAT | | |
| | | RVLSELLEGAGRRRACR | | gttgctcata | | GATGATTACG | | |
| | | AMRPKTA | | acagggacgg | | ACCTGAGTCA | | |
| | | GRRNDLHDDRTASAHKT | | tgtacctgtt | | CTAAAGACGA | | |
| | | SRQKRRAEYARVQELYK | | tgctcgtggc | | TGGCATGATG | | |
| | | KCRSRAA | | tggctatcga | | ATCCGGCGAT | | |
| | | AEVIDGACGGVGHSLEE | | atggacggga | | GAAAA | | |
| | | METYWRPILERVSDAPG | | ccaatacacc | | | | |
| | | PTPEALHA | | cccctgttag | | | | |
| | | LGRAEWHGGNRDYTQLW | | taatggggta | | | | |
| | | KPISVEEIKASRFDWRT | | agagagagcg | | | | |
| | | SPGPDGIR | | gtctgaaact | | | | |
| | | SGQWRAVPVHLKAEMFN | | atggccgaaa | | | | |
| | | AWMARGEIPEILRQCRT | | tcacgacgcc | | | | |
| | | VFVPKVE | | ccactcctac | | | | |
| | | RPGGPGEYRPILIASIP | | ccataacctg | | | | |
| | | LRHFHSILARRLLACCP | | cacgtggtac | | | | |
| | | PDARQRGFIC | | cgccgcacat | | | | |
| | | ADGTLENSAVLDAVLGD | | tgaccgatac | | | | |
| | | SRKKLRECHVAVLDFAK | | gggaggaggg | | | | |
| | | AFDTVSHE | | gcagcacttg | | | | |
| | | ALVELLRLRGMPEQFCG | | aatcacgtag | | | | |
| | | YIAHLYDTASTTLAVNN | | tcttggtgta | | | | |
| | | EMSSPVKVG | | gccattgcgg | | | | |
| | | RGVRQGDPLSPILFNVV | | gactacagcc | | | | |
| | | MDLILASLPERVGYRLE | | ctcgtaagtg | | | | |
| | | MELVSALAY | | ccgccttaga | | | | |
| | | ADDLVLLAGSKVGMQES | | acgcaacggg | | | | |
| | | ISAVDCVGKQMGLRLNC | | gcaataggtg | | | | |
| | | RKSAVLSM | | ggccggggcg | | | | |
| | | IPDGHRKKHHYLTERTF | | ctagcggggg | | | | |
| | | NIGGKPLRQVSCVERWR | | ggagtaatct | | | | |
| | | YLGVDFEA | | cccctgttgg | | | | |
| | | SGCVTLEHSISSALNNI | | cgtgcaccgc | | | | |
| | | SRAPLKPQQRLEILRAH | | actgctccca | | | | |
| | | LIPRFQHGFV | | ctgggggcag | | | | |
| | | LGNISDDRLRMLDVQIR | | tgtcatccgg | | | | |
| | | KAVGQWLRLPADVPKAY | | aaacaggtgg | | | | |
| | | YHAAVQDG | | gccggggcgc | | | | |
| | | GLAIPSVRATIPDLIVR | | caccaggggg | | | | |
| | | RFGGLDSSPWSVARAAA | | gagcaatccc | | | | |
| | | KSDKIRKKLR | | tcctg | | | | |
| | | WAWKQLRRFSRVDSTTQ | | | | | | |
| | | RPSVRLFWREHLHASVD | | | | | | |
| | | GRELRES | | | | | | |
| | | TRTPTSTKWIRERCAQI | | | | | | |
| | | TGRDFVQFVHTHINALP | | | | | | |
| | | SRIRGSRGR | | | | | | |
| | | RGGGESSLTCRAGCKVR | | | | | | |
| | | ETTAHILQQCHRTHGGR | | | | | | |
| | | ILRHNKIV | | | | | | |
| | | SFVAKAMEENKWTVELE | | | | | | |
| | | PRLRTSVGLRKPDIIAS | | | | | | |
| | | RDGVGVIVD | | | | | | |
| | | VQVVSGQRSLDELHREK | | | | | | |
| | | RNKYGNHGELVELVAGR | | | | | | |
| | | LGLPKAE | | | | | | |
| | | CVRATSCTISWRGVWSL | | | | | | |
| | | TSYKELRSIIGLREPTL | | | | | | |
| | | QIVPILALRGS | | | | | | |
| | | HMNWTRFNQMTSVMGGG | | | | | | |
| | | VG* | | | | | | |
| Mesoligia | R2Mes | MGVAFDDNNELYGRTSG | 33483 | ATCTTGTTGC | 33495 | TCGGATTCCT | 33505 | OU744815.1 |
| furuncula | | LPQEPEASTLKPVSPTA | | TCACGTCTGG | | GTGTCGCGGG | | |
| | | RSPPRSG | | GGATGTGTAC | | CCGCCGCCGG | | |
| | | RGEGEWTCPECSRAFRT | | CCTCTTTGCT | | GCCCAGGGGT | | |
| | | KTGLGVHKRRAHPVTAN | | GCCGCTATCA | | CGAAGCTCCC | | |
| | | AAAAPPQ | | GTTAATTGGG | | ACCTTTGGCT | | |
| | | VKRRWLEEEGELLAQTE | | AAAGAGCTGT | | TCACCTGGTT | | |
| | | ARLVRAGGSASTINQQL | | TCACAGACGT | | AATTTTTGTA | | |
| | | MRELPQLG | | CAATGTCCTA | | CCAACTTCGA | | |
| | | RSLEAIKGYRRKEAYKS | | GCGATACGAC | | CCTCGCCAGA | | |
| | | RVQACLADLAQPPSPST | | TAAATGGGGG | | GATCCCACCG | | |
| | | PGEANLPIR | | TTGATGTCGC | | GCGTAATTTC | | |
| | | STPIAGTAESSTPEQPL | | TAGGAAACTC | | GACAGTTAGG | | |
| | | VWAEPPSEVLPVSVSLQ | | CTATCCTTGG | | TGACGGTAGG | | |
| | | SPDLSEALD | | CCTGCCCGTG | | GGACGTGGGA | | |
| | | HVKIVEDLLASSEARMA | | GCGCCGCTGC | | GTCCTGTCAC | | |
| | | RGASDGKRRGRPRRKGQ | | TTCGCGAACA | | AACAATCTGT | | |
| | | SPEETQIA | | AGGGGGGGGG | | GATGATTATA | | |
| | | FARLSARKRRRMEYARV | | GGGGCAATCA | | TTACGTTCAC | | |
| | | QELYKTCRSRAAAEVID | | CTAATATATG | | TAAGACGATG | | |
| | | GQTRGVSH | | GACTGGATAG | | GCACTGTTCG | | |
| | | SLSELEAYWRPVMEAVS | | CAAGGACAGT | | AGC | | |
| | | DAPGLTPEVLGALQRSE | | CCGTTAAAAG | | | | |
| | | QYGGSRD | | CATCTCATGG | | | | |
| | | YSQLWTPFTSDEVKACR | | TGGTAGACCC | | | | |
| | | VDNRSGPGPEGILPGAW | | TAATACAAGT | | | | |
| | | NTLSSAT | | TGAGGTGTCG | | | | |
| | | QAEIFNAWLMAGEVPEK | | CGCATACCTC | | | | |
| | | LRGCRTVFVPKTETPAG | | CCCGACTTGA | | | | |
| | | PGEYRPISI | | CCAGAAGACG | | | | |
| | | ASVPLRHMHSVLAKRLE | | CGAGTCTGAT | | | | |
| | | ACCPPDARQRGFICADG | | GGTCCTAGGG | | | | |
| | | TLENSAVL | | GTACGGTGAG | | | | |
| | | DAVLGDCGKKLRECHVA | | CCTGGAGACC | | | | |
| | | VLDFAKAFDTVSHAALI | | TTGGTCACGA | | | | |
| | | DLLRKRGLP | | AATCGGATCA | | | | |
| | | EGFCNYVARLYDTSETV | | AGGTGCGCAA | | | | |
| | | LVANGARSGPARVGQGV | | ACACGCGAAA | | | | |
| | | RQGDPLS | | GGTGCATATC | | | | |
| | | PLLFNMAMDVILAALPR | | CAGGACAGGC | | | | |
| | | EVGYGLEGENVSALAYA | | TCTGGGGGAA | | | | |
| | | DDLVLLAGS | | GGTCTCAGAG | | | | |
| | | KVGMQSSIDCVWRTGRM | | AAGCCCCGAT | | | | |
| | | MGLFISHAKSAVLSMVP | | GGGAGTCCTT | | | | |
| | | DGKRKKV | | GGGCTCCAAA | | | | |
| | | HFLTDRTFKVGSRWLRQ | | TCTAGCAGCT | | | | |
| | | VSCVERWRYLGVDFKAS | | CGCATGGCTG | | | | |
| | | GCVTLEH | | GGGCGTACAC | | | | |
| | | DVKVALNNITKAPLKPQ | | GAGAGCTGTG | | | | |
| | | QRLEILRVHLIPRFLHG | | GGTGAACCTG | | | | |
| | | FVLGIITDDRL | | TGGATAGGGC | | | | |
| | | RMLDVQIRRAVRTWLRL | | TAGCCACCCT | | | | |
| | | PKDVPVGYFHAATADGG | | ACCACAATAG | | | | |
| | | LAIPSLRT | | GTATTAAGGT | | | | |
| | | CVPDLIKKRFGRLDSSR | | GTTGCTTTCG | | | | |
| | | WPVARAAARSERIRRKL | | ATGATAATAA | | | | |
| | | QWADKQLR | | TGAGTTGTAT | | | | |
| | | KFTAENPKSGERTTAMY | | GGTCGAACCT | | | | |
| | | WREALHASVDGLELREC | | CCGGCCTCCC | | | | |
| | | PRVPASTK | | ACAGGAGCCG | | | | |
| | | WMRERSMQYTGRDFVQF | | GAGGCGTCGA | | | | |
| | | VHTHINALPSRVRNTRG | | CCCTTAAACC | | | | |
| | | RRTGVAS | | TGTAAGTCCG | | | | |
| | | ELNCRAGCMVRETTAHT | | ACTGCTCGAT | | | | |
| | | IQQCHRTHGGRIKRHNC | | CTCCGCCTCG | | | | |
| | | VADVVCSA | | GTCGGGGCGG | | | | |
| | | MEDKGWTVVKEPKVRTA | | GGGGAAGGCG | | | | |
| | | LGLRKPDIIASRNGVGV | | AGTGGACCTG | | | | |
| | | IVDAQVVS | | TCCGGAATGT | | | | |
| | | GQRPLDELHREKRNKYG | | AGCAGGGCAT | | | | |
| | | NHAELVEKVADILGLPC | | TCCGGACCAA | | | | |
| | | KESVHSTS | | AACAGGGTTG | | | | |
| | | CTLSWRGVWSLASYREL | | GGCGTGCACA | | | | |
| | | KRFVGLDEGVLAGVPSL | | AGCGGCGCGC | | | | |
| | | VLRGSHIN | | ACACCCTGTG | | | | |
| | | WTRFNRMTTVSTESGSE | | ACGGCAAACG | | | | |
| | | | | CCGCAGCCGC | | | | |
| | | | | CCCACCGCAG | | | | |
| | | | | GTGAAGCGGA | | | | |
| | | | | GATGGCTTGA | | | | |
| | | | | AGAAGAGGGT | | | | |
| | | | | GAGCTTCTTG | | | | |
| | | | | CGCAGACGGA | | | | |
| | | | | GGCGCGTCTG | | | | |
| | | | | GTTAGAGCCG | | | | |
| | | | | GAGGCAGTGC | | | | |
| | | | | CAGTACAATT | | | | |
| | | | | AACCAGCAAT | | | | |
| | | | | TA | | | | |
| Cionaintes | R2Ci | MGEWPWVSWSLTVLVEK | 33484 | CGACGGTGAA | 33496 | TGACAGTAAT | 33506 | AB097122.1 |
| tinalis | | WRPFTILQPYPMPGQLR | | CCACCTTGTC | | ATGAAAACAT | | |
| | | VDVYLPR | | GCGGTGTAAG | | CACATCTGAC | | |
| | | KTSYLMDKNIYENTTSP | | AGCTTTAGTG | | CGGCACAGAA | | |
| | | GGGPLCGEKTHRSDVII | | TCTCGAACAA | | TCACCATGCC | | |
| | | PPPGFAPST | | GAAATAGCTT | | GTAATGCACC | | |
| | | DTASNTLGENVDASATT | | GTGTGCTGTC | | CAACTAAGGA | | |
| | | SSANPLSQEPGWCESCS | | CTTCTGGGCG | | TTCCAATGGG | | |
| | | KLFKSQR | | GTGCACATAC | | TAAAAAAAAA | | |
| | | GLRVHQRSKHPELYHSQ | | TTCTTAACCT | | AAAAAAAAAA | | |
| | | NQPLPRSKARWSDEEMV | | CCCGAGGCCA | | AAAAAAAAAA | | |
| | | IFAREELA | | TGCCGGCGGG | | AAAAAAAAAA | | |
| | | NRKIRFINQHLHKVFPH | | GGCTTTAGCC | | AA | | |
| | | RTLESIKGLRGKNVRYA | | CCCGGCAGGT | | | | |
| | | RIMADLEAEM | | TTTACCATGC | | | | |
| | | TSQPEAATSLCTETSEN | | CGGACGGGTT | | | | |
| | | LASSNVLPQTRGWAENL | | CGAGAGGTAG | | | | |
| | | VENIDTAHL | | AGGCCAAACT | | | | |
| | | ANLGPLSQFEPGKPSSS | | AAGAGTTCAC | | | | |
| | | TKEAINTEYNDWISKWL | | CAGCAGACTT | | | | |
| | | PSGAAHRE | | CGCACGCGGC | | | | |
| | | RRANPPSTKLNARATRR | | TGGCCACTGG | | | | |
| | | LQYSRIQNLYKLNRSAC | | CCGAAGTTTA | | | | |
| | | AQEVLSGA | | AACAACAGGG | | | | |
| | | WKVQSGELNLKEVQPFW | | CCGCATCTTC | | | | |
| | | EKMFRKESAKDRRKPKP | | CCAAACTCAA | | | | |
| | | TGEVLW | | TATATGGTGT | | | | |
| | | GLMEPLTIAEVGSTLKS | | TAAGTGAACC | | | | |
| | | TTPSAPGPDKLTLDGVK | | GTGCCG | | | | |
| | | RIPIAELVSH | | | | | | |
| | | YNLWLYAGYQPEGLREG | | | | | | |
| | | ITTLIPKIKGTRDPAKL | | | | | | |
| | | RPITVSSFICR | | | | | | |
| | | IFHRCLAQRMETSLPLG | | | | | | |
| | | ERQKAFRKVDGICHNIW | | | | | | |
| | | SLRSLIHNS | | | | | | |
| | | KDNLKELNITFLDVRKA | | | | | | |
| | | FDSISHKSLGIAAARLG | | | | | | |
| | | LPPPLITYISNL | | | | | | |
| | | YPNCSTKLKVNGKISKP | | | | | | |
| | | IEVRRGVRQGDPLSPLL | | | | | | |
| | | FNAVMDWA | | | | | | |
| | | LSELDPRVGVQIGEQRI | | | | | | |
| | | NHLAFADDIILVSSTKI | | | | | | |
| | | GMVSSINTLSR | | | | | | |
| | | HLAKSGLEISAGKEGKS | | | | | | |
| | | ASMAIVVDGKKKMWTVD | | | | | | |
| | | PLPRFKVN | | | | | | |
| | | SQKIPALSITQQYKYLG | | | | | | |
| | | INIDAQGARNDAARILT | | | | | | |
| | | EGLAELSRAPL | | | | | | |
| | | KPQQRLYLLRVHLLPKL | | | | | | |
| | | QHGLVLSSCAKRALTYL | | | | | | |
| | | DKSVRSAIR | | | | | | |
| | | RWLTLPKDTPTAFYHAK | | | | | | |
| | | ACDGGLGITRLEHTIPI | | | | | | |
| | | LKRNRMMKL | | | | | | |
| | | TLSEDPVIMELVKLTYF | | | | | | |
| | | TNLLHKYSNVKLLNSWP | | | | | | |
| | | VTDKDSLAR | | | | | | |
| | | AEASMLHTSVDGRGLSN | | | | | | |
| | | CSDVPRQSDWVTNGASL | | | | | | |
| | | LSGRDFI | | | | | | |
| | | GAIKVRGNLLPTKVSAA | | | | | | |
| | | RGRQREITCDCCRRPES | | | | | | |
| | | LGHILQTCP | | | | | | |
| | | RTWGPRISRHDSLLKRV | | | | | | |
| | | RNQACLKNWTPIIEPSI | | | | | | |
| | | PTNIGLRRPD | | | | | | |
| | | LVLAKGNIAFLVDATVV | | | | | | |
| | | ADNANMQLQHEAKVEKY | | | | | | |
| | | NNSDIKEWI | | | | | | |
| | | KVHCPGVDEVRVTSLTA | | | | | | |
| | | NWRGCLYGGSASFLTED | | | | | | |
| | | LGLPKAEL | | | | | | |
| | | SLLSAKINEKGYYLWCA | | | | | | |
| | | HYRGTARLWNRPLRS | | | | | | |
| Ixodes | R21s | MQCTSRLADAPRFARVG | 33485 | GTTCCAAAGG | 33497 | TAGTGTGACG | 33507 | GCA_016920785.2 |
| scapularis | | VEGEGVGASGNGTDAQL | | AAGGCACTCC | | GAGTCCTCAA | | |
| | | WYGCTG | | TTTGGTTCGT | | GCCCCCACAA | | |
| | | CDEAFSSLRGLRIHAAQ | | GATGAGATGT | | GTGCCTGCCA | | |
| | | KKHGNQDGLLRLPAGRP | | TCATGGTGCT | | GGTGGCAGGA | | |
| | | RKRRVGKS | | TGCCTAGCTG | | AAGGGCAACT | | |
| | | TTAGASDRVTTDPVPAP | | GAGAAATCCG | | ACTGGTGAGC | | |
| | | VPESPGLLPGLPGPSLP | | ACTCACACCT | | GACCCAAGCA | | |
| | | GCSDLPPG | | GCACGTGGTC | | AGGCGGAGCC | | |
| | | VLPGGWSASPGPLSWPP | | CCTGCCGCCT | | AAGACCAAGC | | |
| | | SLDAGPLPGPSRVSPGP | | GCCAGTATGC | | TGGAGCCAAG | | |
| | | SRPSPGK | | CGAGGAAACG | | AGCAACTCCA | | |
| | | PTGPPSLDAGPLPGPSR | | GGTGCAACTT | | GGAGGCAGGG | | |
| | | VSPGPSRPSPGKPPGTP | | AATCCGTGGA | | GTGGATATCA | | |
| | | EPLPGSP | | TACTGGTAGC | | AGAGCAACCC | | |
| | | GGRRGVSPGQPGSRTDP | | AACGTGAGCA | | CAAGGGACAC | | |
| | | SSSAGAGHFVCPQCSRA | | ACGGTACGGT | | AGACCACGGG | | |
| | | FSSKIGM | | CCTTCGCGGA | | CAACTACTGG | | |
| | | SQHQKHAHLEEYNAGIN | | CCACCCTGGG | | TGAGCGCCCA | | |
| | | ITRTKARWDPEETYLLA | | CGTTCGGGTT | | AGACAGGGGT | | |
| | | RLEATLNPD | | GCCAGCCCGT | | GGATATTAAG | | |
| | | HKNINQTLHAALPRGSC | | TCGCCCGAAA | | AACAGCCCCA | | |
| | | RTLESIKAHRKQAAYRD | | TATCTTGGCC | | CAAAGTGTTA | | |
| | | LVTSLRSAR | | CTGAAACTAA | | CCTATATTAA | | |
| | | ESSEAQHVPDRPLETPE | | AAGAAAA | | CAATAAAGTT | | |
| | | PQTPANPQRDSKQAVIE | | | | GAAGCCTCAA | | |
| | | ALQSLIGRA | | | | CCACGCATTG | | |
| | | PPGSFQGARLWDIARQA | | | | CGGGTTAGAT | | |
| | | TRGTNILPLLNSYLRDV | | | | GGCGTGGCTT | | |
| | | FTLPTKPTR | | | | GGCCCGCCGC | | |
| | | KKPAVRPARSRRKQKKQ | | | | CATGATGAGC | | |
| | | EYARTQDLFRKKQSDCA | | | | TGGAACCCTC | | |
| | | RAVLDGP | | | | CACCTGGTGG | | |
| | | TSSSVPGTGAFLQTWRE | | | | GCCGCACGAG | | |
| | | IMTGPSPALEAPPLPTR | | | | ACCACCGGCT | | |
| | | GEVDLFFPA | | | | CTTTCTACTA | | |
| | | TAQEIQSAEIAVNSAAG | | | | AGGCCGGTCT | | |
| | | PDGFSARLLKSVPALLL | | | | CCGTGACTGC | | |
| | | RVMVNLLLLV | | | | GGTTGGGATA | | |
| | | RRVPAALRDARTTFIPK | | | | AACTCCAAGC | | |
| | | VPDAVDPSQFRPITVAS | | | | ACTGAGCGGT | | |
| | | VLQRLLHRIL | | | | AAAAAAAAAA | | |
| | | AKRALEAIPLNFRQRAF | | | | AAAAAAAAAA | | |
| | | QPVDGCAENIWLLSTAL | | | | AAAAAAA | | |
| | | NEARTRRRP | | | | | | |
| | | LHMASVDLTKAFDRVTT | | | | | | |
| | | DAILRGARRAGLSGEFI | | | | | | |
| | | GYLKELYTTS | | | | | | |
| | | RTLLQFQGESLLVEPTT | | | | | | |
| | | GVRQGDPLSPILFNLVL | | | | | | |
| | | DEYLSSLDP | | | | | | |
| | | DISFVSGDLRLDAMAFA | | | | | | |
| | | DDLIVFASTPAGLQDRL | | | | | | |
| | | DALVEFFDP | | | | | | |
| | | RGLRVNVKKSFTLSLQP | | | | | | |
| | | GRDKKVKVVCDQIFTIG | | | | | | |
| | | GTPLPASKV | | | | | | |
| | | ATPWRYLGMTFTPQGSI | | | | | | |
| | | NKGTSEQLDLLLTRTSK | | | | | | |
| | | APLKPQQR | | | | | | |
| | | LVVLRNYLLPRLYHRLV | | | | | | |
| | | LGPWSAALLLKMDTTIR | | | | | | |
| | | GAIRRWMDL | | | | | | |
| | | PHDTPLGFFHAPVTEGG | | | | | | |
| | | LGINSLRASIPAMVLQR | | | | | | |
| | | LDGLHFSTH | | | | | | |
| | | PGAEVAIQLPFLTGLHR | | | | | | |
| | | RAEAAAQYQGQRLLSKA | | | | | | |
| | | DVHRMWSA | | | | | | |
| | | RLHGSCDGRPLRESKRV | | | | | | |
| | | PAAHRWAAEGTRLLSGR | | | | | | |
| | | DFISITKLK | | | | | | |
| | | INALPTLERTSRGQHKD | | | | | | |
| | | IQCRAGCQAVESLGHVL | | | | | | |
| | | QACHRGHR | | | | | | |
| | | GRIRRHDNIARYVCGRL | | | | | | |
| | | TQIGWAVKWEPHYSVAG | | | | | | |
| | | RTLKPDIVA | | | | | | |
| | | HRGAETVVLDAQVVGTS | | | | | | |
| | | MRLGFHHAQKKEKYSLP | | | | | | |
| | | DLLHQVC | | | | | | |
| | | EGRRDAARVSTITLNFR | | | | | | |
| | | GVWAPESAQDLKSLGLT | | | | | | |
| | | DNDLKLLTV | | | | | | |
| | | RCLQGGAQCFRLHRRMT | | | | | | |
| | | TVVKATGDEANALPAHS | | | | | | |
| | | GLPPTQL | | | | | | |
| | | GGRTLGPSAHNQSARTT | | | | | | |
| Trichinella | R2TsP | MSNRLANTAAAGGVPEK | 33486 | CTCCTGACTA | 33498 | TGAGGTTTTT | 33508 | CP032378.1 |
| spiralis | | TSGTLDIPGQPSSSGEK | | ACCTGATTTC | | GTTTTCTTTT | | |
| | | RAISYPGP | | GTCCGTGCGG | | TTCCTTTTAC | | |
| | | FGCNSCSFTSTTWLSLE | | CGGCGTTTTC | | CATTCTTGTT | | |
| | | LHFKSVHNIRDFVFLCS | | TTTTCGCTCT | | CCATTGTTGT | | |
| | | KCKKSWPSI | | CCGCTCGTCG | | TATTTGCTTT | | |
| | | NSVASHYPRCKGSVKAA | | AAATTTGCTG | | AATCCTGTAT | | |
| | | VVPTSLANTCTTCGSSF | | TAGTTGATTC | | TTTACCGCCG | | |
| | | GTFSGLQL | | GCTTTTCTTT | | GCAATTCCAT | | |
| | | HRKRAHPDVFAASCSKK | | GCGTTTTCTT | | TGTTATTATT | | |
| | | TKARWSNDEFTLLARLE | | CTACTTTCGC | | ACTGTTACTG | | |
| | | AGLDPACK | | AGTTTTTTCT | | TTATTATTGT | | |
| | | NINQVLAERLMEYNITR | | GCATTGCCAC | | TACTATTGTT | | |
| | | GVEMIKGQRRKDQYKAL | | G | | TTTACTTTTA | | |
| | | VRQLRSNS | | | | CTTACTACTG | | |
| | | ETQQCVGLAGSMDSNVP | | | | TTATTATACT | | |
| | | ANDTSSSVASEVSITYP | | | | TTAATTCGTT | | |
| | | EYGAVMSC | | | | AACTTACGTT | | |
| | | DLIKEATGMAIVDINEL | | | | ATTGTTACCA | | |
| | | QSNLRKAFLSGRKLPMK | | | | CTACTTACTT | | |
| | | FHGARETAQ | | | | TGCTCTCTCG | | |
| | | KKMANPRVAKFKRFQRL | | | | CAAACGTTCG | | |
| | | FRSNRRKLASHIFDKAS | | | | TTGTTGTTTC | | |
| | | LEQFGGSID | | | | TTTTGGACCA | | |
| | | EASDHLEKFLSRPRLES | | | | GGTTTAGAGA | | |
| | | DSYSVISGDKSIGVAHP | | | | AATCGCACGC | | |
| | | ILAEEVELEL | | | | ACAGCGGAAC | | |
| | | KASRPTAVGPDGIALED | | | | TGGACCGCTT | | |
| | | IKKLNTYDIASLFNLWL | | | | AAGCCAGAAA | | |
| | | KAGDLPASVK | | | | TAGTAAAGTA | | |
| | | ASRTIFLPKSDGTTDIS | | | | ACAA | | |
| | | NCRPITIASAMYRLFSR | | | | | | |
| | | IITRRLAARLEL | | | | | | |
| | | NVRQKAFRPEMNGVFEN | | | | | | |
| | | SAILYALIKDAKVRSRE | | | | | | |
| | | ICVTTLDLAK | | | | | | |
| | | AFDTVPHSRILRALRKN | | | | | | |
| | | NVDPESVDLISKMLTGT | | | | | | |
| | | TYAEIKGLQG | | | | | | |
| | | KLIPIRNGVRQGDPLSP | | | | | | |
| | | LLFSLFIDEIIGRLQAC | | | | | | |
| | | GPAYDFHGEKI | | | | | | |
| | | CILAFADDLTLVADSAA | | | | | | |
| | | GMKILLKAACDFLEESG | | | | | | |
| | | MSLNAEKCR | | | | | | |
| | | TLCITRSPRSRKTFVNP | | | | | | |
| | | AAKFIISDWKTGISSEI | | | | | | |
| | | PSLCATDTFRF | | | | | | |
| | | LGHTFDGEGKIHIDTEE | | | | | | |
| | | IRSMLKSVKSAPLKPEQ | | | | | | |
| | | KVALIRSHLL | | | | | | |
| | | PRLQFLFSTAEADSRKA | | | | | | |
| | | WLIDSIIRGCVKEILHS | | | | | | |
| | | VKAGMCTDIF | | | | | | |
| | | YIPSRDGGMGFTSLGEF | | | | | | |
| | | SLFSRQKALAKMAGSSD | | | | | | |
| | | PLSKRVAE | | | | | | |
| | | FFIERWNIARDPKVIEA | | | | | | |
| | | ARRVYQKKRYQRFFQTY | | | | | | |
| | | QSGGWNEF | | | | | | |
| | | SGNTIGNAWLTNGRARG | | | | | | |
| | | RNFIMAVKFRSNTAATR | | | | | | |
| | | AENLRGRP | | | | | | |
| | | GTKECRFCKSATETLAH | | | | | | |
| | | ICQRCPANHGLVIQRHD | | | | | | |
| | | AWTFLGEV | | | | | | |
| | | ARKEGYQVMIEPKVSTP | | | | | | |
| | | VGALKPDLLLIKADTAF | | | | | | |
| | | IVDVGIAWEG | | | | | | |
| | | GRPLKLVNKMKCDKYKT | | | | | | |
| | | AIPAILETFHVGHAETY | | | | | | |
| | | GVILGSRGC | | | | | | |
| | | WLKSNDKALASIGLNIT | | | | | | |
| | | RKMKEHLSWLTFEIIFI | | | | | | |
| | | TQISRIYNSFMKK | | | | | | |
| Taeniopygia | R2Tg | MASCPKPGPPVSAGAMS | 33487 | GTCTAGTTAC | 33393 | TTCAGGTTAT | 33509 | XR_005978890.1 |
| guttata | | LESGLTTHSVLAIERGP | | AACTGGGCAT | | TTAGATGCTT | | |
| | | NSLANSGS | | CGCTGCAGAG | | AGTTTTTGTA | | |
| | | DFGGGGLGLPLRLLRVS | | ATCGCACCTC | | CCTTTCTTGT | | |
| | | VGTQTSRSDWVDLVSWS | | CTCGTGGTCC | | TTTGTTTAGG | | |
| | | HPGPTSK | | CGCTGGTAGC | | ATTTTGATAG | | |
| | | SQQVDLVSLFPKHRVDL | | CCTTCGAAGG | | TGTTAGTATT | | |
| | | LSKNDQVDLVAQFLPSK | | GTGACTAAGT | | TTTATATTTT | | |
| | | FPPNLAEN | | CGATCTCTGC | | TGTACGATTG | | |
| | | DLALLVNLEFYRSDLHV | | CCCAGGTACG | | CATAATGTTC | | |
| | | YECVHFAAHWEGLSGLP | | GAGCCGTTGG | | TTTTTTATAC | | |
| | | EVYEQLAP | | GACTCACCAG | | AGTTCTGTTT | | |
| | | QPCVGETLHSSLPRDSE | | TCCAACGTAA | | TAATAAAATA | | |
| | | LFVPEEGSSEKESEDAP | | CTCCTGCCTA | | GACGATAGCT | | |
| | | KTSPPTPG | | AATTCGGTGA | | AGAGACGTTA | | |
| | | KHGLEQTGEEKVMVTVP | | AACAAATTCC | | GGGCAGCCAC | | |
| | | DKNPPCPCCGTRVNSVL | | TCGGTAAAAA | | AAGCCAGTTA | | |
| | | NLIEHLKV | | GCCCC | | GGTAGCGGAT | | |
| | | SHGKRGVCFRCAKCGKE | | | | AGTAGGTAGG | | |
| | | NSNYHSVVCHFPKCRGP | | | | AACAGACTTT | | |
| | | ETEKAPA | | | | TACTATTTCA | | |
| | | GEWICEVCNRDFTTKIG | | | | TAACGCGTCA | | |
| | | LGQHKRLAHPAVRNQER | | | | ATTACCACCT | | |
| | | IVASQPKE | | | | GATTTGGACC | | |
| | | TSNRGAHKRCWTKEEEE | | | | AATTCACGGG | | |
| | | LLIRLEAQFEGNKNINK | | | | ATTTGTCCAA | | |
| | | LIAEHITTKT | | | | GGTGGACGGG | | |
| | | AKQISDKRRLLSRKPAE | | | | CCACCTTTAC | | |
| | | EPREEPGTCHHTRRAAA | | | | TTAACCCGGA | | |
| | | SLRTEPEM | | | | AAAGGAACAT | | |
| | | SHHAQAEDRDNGPGRRP | | | | ATATAATTTA | | |
| | | LPGRAAAGGRTMDEIRR | | | | TGTGTGTTCG | | |
| | | HPDKGN | | | | ATAAA | | |
| | | GQQRPTKQKSEEQLQAY | | | | | | |
| | | YKKTLEERLSAGALNTF | | | | | | |
| | | PRAFKQVM | | | | | | |
| | | EGRDIKLVINQTAQDCF | | | | | | |
| | | GCLESISQIRTATRDKK | | | | | | |
| | | DTVTREKHPK | | | | | | |
| | | KPFQKWMKDRAIKKGNY | | | | | | |
| | | LRFQRLFYLDRGKLAKI | | | | | | |
| | | ILDDIECLSC | | | | | | |
| | | DIPLSEIYSVFKTRWET | | | | | | |
| | | TGSFKSLGDFKTYGKAD | | | | | | |
| | | NTAFRELITA | | | | | | |
| | | KEIEKNVQEMSKGSAPG | | | | | | |
| | | PDGITLGDVVKMDPEFS | | | | | | |
| | | RTMEIFNL | | | | | | |
| | | WLTTGKIPDMVRGCRTV | | | | | | |
| | | LIPKSSKPDRLKDINNW | | | | | | |
| | | RPITIGSILLR | | | | | | |
| | | LFSRIVTARLSKACPLN | | | | | | |
| | | PRQRGFIRAAGCSENLK | | | | | | |
| | | LLQTIIWSAK | | | | | | |
| | | REHRPLGVVFVDIAKAF | | | | | | |
| | | DTVSHQHIIHALQQREV | | | | | | |
| | | DPHIVGLVSN | | | | | | |
| | | MYENISTYITTKRNTHT | | | | | | |
| | | DKIQIRVGVKQGDPMSP | | | | | | |
| | | LLFNLAMDPL | | | | | | |
| | | LCKLEESGKGYHRGQSS | | | | | | |
| | | ITAMAFADDLVLLSDSW | | | | | | |
| | | ENMNTNISI | | | | | | |
| | | LETFCNLTGLKTQGQKC | | | | | | |
| | | HGFYIKPTKDSYTINDC | | | | | | |
| | | AAWTINGTP | | | | | | |
| | | LNMIDPGESEKYLGLQF | | | | | | |
| | | DPWIGIARSGLSTKLDF | | | | | | |
| | | WLQRIDQAP | | | | | | |
| | | LKPLQKTDILKTYTIPR | | | | | | |
| | | LIYIADHSEVKTALLET | | | | | | |
| | | LDQKIRTAVKEW | | | | | | |
| | | LHLPPCTCDAILYSSTR | | | | | | |
| | | DGGLGITKLAGLIPSVQ | | | | | | |
| | | ARRLHRIAQS | | | | | | |
| | | SDDTMKCFMEKEKMEQL | | | | | | |
| | | HKKLWIQAGGDRENIPS | | | | | | |
| | | IWEAPPSS | | | | | | |
| | | EPPNNVSTNSEWEAPTQ | | | | | | |
| | | KDKFPKPCNWRKNEFKK | | | | | | |
| | | WTKLASQ | | | | | | |
| | | GRGIVNFERDKISNHWI | | | | | | |
| | | QYYRRIPHRKLLTALQL | | | | | | |
| | | RANVYPTREF | | | | | | |
| | | LARGRQDQYIKACRHCD | | | | | | |
| | | ADIESCAHIIGNCPVTQ | | | | | | |
| | | DARIKRHNYI | | | | | | |
| | | CELLLEEAKKKDWVVFK | | | | | | |
| | | EPHIRDSNKELYKPDLl | | | | | | |
| | | FVKDARALW | | | | | | |
| | | DVTVRYEAAKSSLEEAA | | | | | | |
| | | AEKVRKYKHLETEVRHL | | | | | | |
| | | TNAKDVTFV | | | | | | |
| | | GFPLGARGKWHQDNFKL | | | | | | |
| | | LTELGLSKSRQVKMAET | | | | | | |
| | | FSTVALFS | | | | | | |
| | | SVDIVHMFASRARKSMV | | | | | | |
| | | M | | | | | | |
| Talpaocci- | R2ToC | MLAPRSDRGNGFGDGPA | 33488 | TCTAGTTACA | 33395 | TGACTGTTTA | 33510 | NW_023605038.1 |
| dentalis | | THPVPVNEIGQEPIDPD | | ACTGGGCATA | | GAGTAGGATT | | |
| | | PFLGGENC | | GCTGCAGAGA | | TTTTATTTGA | | |
| | | GLPLRLFGVSVGTQTSQ | | TCTCACCTCC | | TATTATGTAT | | |
| | | EDLTPIPTKLAVNELDV | | TCGTGGTCCC | | GTTTTATACC | | |
| | | LVNFSFEVY | | GCTGGTAAGC | | TTGTACTTTG | | |
| | | RSDLKGYVGGVHFPVNL | | CCTTAACAGG | | TTCATTTATA | | |
| | | EVLEGFPEVYEHLEPQP | | GTGACTAAGT | | TTGTATTGGG | | |
| | | CQGDNLD | | AGATCTCTGC | | GGGATTTTTT | | |
| | | PSPPDDGVQVVLGREEG | | CCCAGTCAAG | | GTAGCATGGG | | |
| | | KKEREGAPEALPPVQRG | | GAGCCGCTGG | | ATTGTTTTTA | | |
| | | HSEQVPD | | GAATCACCAG | | TTGTATGACC | | |
| | | DIVKVTVPDKNPPCPCC | | CCCAGCGATT | | TTTTTGATAT | | |
| | | STRLNSVLALIDHLKGS | | CCTTTCAAAT | | TTTTAATAAA | | |
| | | HGKRRVCFR | | TTAGGTGAAA | | CTAGACGGTA | | |
| | | CAKCGRENFNHHSTVCH | | CAAATTTCTC | | GCTATGGGGG | | |
| | | FAKCKGPSEEKPPVGEW | | GGTGTGGGTC | | TTAGGGCACG | | |
| | | ICEVCGR | | GCAAGACTTA | | CCACAAGCCA | | |
| | | DFTTKIGLGQHKRLAHP | | CTACCTAAAA | | GTTAGGGCGC | | |
| | | MVRNQERIDASQPKETS | | CCTGGCCCCA | | TCATAGTGAG | | |
| | | NRGAHKKC | | CGGTCTGACA | | TAGGGACAGT | | |
| | | WTKEEEELLARLEVQFE | | GGGGCAACGG | | AATTTTAATT | | |
| | | GHKNINKLIAEHITTKT | | GTTCGGAGAT | | CACAACGCGT | | |
| | | NKQISDKRRQ | | | | CAATTACCAT | | |
| | | MTRKDKGEGGAAGKLGP | | | | CTGATTCGGA | | |
| | | DTGRGNHSQAKVGNNGL | | | | CCAATCTTAC | | |
| | | GGNQLP | | | | CTGACTTGTA | | |
| | | GGPAATKDKAGCHLDKE | | | | CTAAGTTACC | | |
| | | EGNRIAISQQKKGRLQG | | | | GGATTTGTCC | | |
| | | RYHKEIKR | | | | CAGGTGGACG | | |
| | | RLEEGVINTFTKAFKQL | | | | GGCCACCTTT | | |
| | | LECQEVQPLINKTAQDC | | | | ACTTAACCCG | | |
| | | FGLLESACHI | | | | GAAAAGGAAC | | |
| | | RTALRGKNKKETQEKPT | | | | ATGTATTTTA | | |
| | | GGQCLKWMKKRAVKKGN | | | | TATATGTGTT | | |
| | | YLRFQRL | | | | | | |
| | | FHLDRGKLARIILDDIE | | | | | | |
| | | CLSCDIAPSEIYSVFKA | | | | | | |
| | | RWETPGQFAGL | | | | | | |
| | | GNFKSTGKADNKAFSDL | | | | | | |
| | | ITAKEIKKNVQEMSKGS | | | | | | |
| | | APGPDGIAI | | | | | | |
| | | GDIKGMDPGYSRTAELF | | | | | | |
| | | NLWLTSGEIPDMVRGCR | | | | | | |
| | | TVLIPKSTQ | | | | | | |
| | | PERLKDINNWRPITIGS | | | | | | |
| | | ILLRLFSRIITARMTKA | | | | | | |
| | | CPLNPRQRGFI | | | | | | |
| | | RAAGCSENLKLLQTIIR | | | | | | |
| | | TAKSEHRPLGVVFVDIA | | | | | | |
| | | KAFDTVSHQH | | | | | | |
| | | ILHVLQQRGVDPHIIGL | | | | | | |
| | | VSNMYKDISTFVTTKKD | | | | | | |
| | | THTDKIQIRVG | | | | | | |
| | | VKQGDPLSPLLFNLAMD | | | | | | |
| | | PLLCKLEESGNGFHRGG | | | | | | |
| | | HTITAMAF | | | | | | |
| | | ADDLVLLSDSWENMEKN | | | | | | |
| | | IEILEAFCDLTGLKTQG | | | | | | |
| | | QKCHGFYIK | | | | | | |
| | | PTKDSYTVNNCAAWTIY | | | | | | |
| | | GTPLNMINPGDSEKYLG | | | | | | |
| | | LQIDPWTGI | | | | | | |
| | | ARSNISSKLDSWLERIN | | | | | | |
| | | QAPLKPLQKLDILKTYT | | | | | | |
| | | IPRLTYMVDH | | | | | | |
| | | SEMKAGALEALDLQIRS | | | | | | |
| | | AVKDWLHLPSCTCDAIL | | | | | | |
| | | YVSTKDGGL | | | | | | |
| | | GVTKLAGLIPSIQARRL | | | | | | |
| | | HRIAQSPDETMKAFLDK | | | | | | |
| | | EQMEKQYAK | | | | | | |
| | | LWVQAGGKREKIPSIWD | | | | | | |
| | | ALPTPVLLTTSDTLSEW | | | | | | |
| | | EAPNPKSKY | | | | | | |
| | | PRPCNWRRKEFEKWTKL | | | | | | |
| | | QCQGRGIQNFKGDVISN | | | | | | |
| | | NWIQNYR | | | | | | |
| | | RIPHRKLLTAVQLRANV | | | | | | |
| | | YPTREFLGRGRGDDCVK | | | | | | |
| | | FCRHCEVD | | | | | | |
| | | LETCGHIISYCPVTKEA | | | | | | |
| | | RIKRHNRICERLIEEAE | | | | | | |
| | | KKDWWFKEP | | | | | | |
| | | HIRDAVKELFKPDLIFV | | | | | | |
| | | KEDRALVVDVTVRFEAT | | | | | | |
| | | TTSLEEAAIEK | | | | | | |
| | | VDKYKRLETEVRSLTNA | | | | | | |
| | | KDVLFMGFPLGARGKWY | | | | | | |
| | | QGNFKLLD | | | | | | |
| | | MLGLSESRQVTVAKTLS | | | | | | |
| | | TDALISSVDIVHMFASK | | | | | | |
| | | ARKMNLVTV | | | | | | |
| Daniorerio | R2Dr | MESTAKGKSYWMARRPV | 33489 | AATCCCCCCT | 33499 | AAATCCCAGC | 33511 | AB097126.1 |
| | | EGATEGSLGRVPFVTRD | | ACCCAATCCC | | GGGATACAGC | | |
| | | PKRKPEA | | CCCGTCGTGA | | AAGAAGGTAT | | |
| | | KRTLTHGLGLRECSVVL | | CCTCCAGGCC | | CGGATCTAAT | | |
| | | TRLIEGRRGRDHTPSGW | | AGGAATCACG | | AAGGTTGAGC | | |
| | | NAQRGMP | | AGCGTACGAC | | GAGGAGAGGG | | |
| | | NDESSVEEPNGPIPSNP | | AGTGGCCATC | | TGGAGATCCT | | |
| | | IPTGTQALPEPMADGEQ | | CGGCAATGAC | | TTGGGGGGGG | | |
| | | GEHPGVW | | AATAGCGTGA | | TCGGGCTAAG | | |
| | | TLPLRDLNCPLCGGSAS | | CTAACGACAA | | TTCCCCTCTC | | |
| | | TAVKVQRHLAFRHGTVP | | TGAGTCAGAT | | GGGTCCTCCC | | |
| | | VRFSCESC | | CCATGACCCT | | ACGGTGACGC | | |
| | | GKTSPGCHSVLCHIPKC | | TGGAGTGGGT | | TCTACCCCTC | | |
| | | RGPTGEPPEKVVKCEGC | | TAACCTCCGC | | CCTCCTCGCT | | |
| | | SRTFGTRR | | CTCTTTAAAA | | CGTAGAACCC | | |
| | | ACSIHEMHVHSEIRNRK | | AC | | AACGGTGAAC | | |
| | | RIAQDRQEKGTSTDGEG | | | | ACGGTTGGCA | | |
| | | RAGVERAD | | | | GGATGAAGTG | | |
| | | AGEGPSGEGIPPKRPRR | | | | ACGTGAGGGG | | |
| | | ARTPREPSEPPANPPIL | | | | TAAGACATGC | | |
| | | SPQPDLPP | | | | GTACGTGAGC | | |
| | | GGLRDLLREVASGWVRA | | | | GCGCATTTTT | | |
| | | ARDGGTVIDSVLAAWLD | | | | GCTGTTCTCT | | |
| | | GNDRLPE | | | | GGACTGGGTT | | |
| | | LVDAATQRTLQGLPAGR | | | | TCGTCCCCCT | | |
| | | LARRPATFVAPNRRRGR | | | | CACAACCATC | | |
| | | WGRRLKL | | | | ACTTACACTA | | |
| | | LAKRRAYHDCQIRFRKD | | | | TAGGGGCACA | | |
| | | PARLAANILDGKSETSC | | | | GCGGCTCCTA | | |
| | | PINEQAIHEH | | | | CCTCCCTCCC | | |
| | | FRNKWANPSPFGGLGRF | | | | TATGACCCCC | | |
| | | GTENRANNAHLLGPISK | | | | CCTTCCCATA | | |
| | | SEVQTSLR | | | | CCGATCCATG | | |
| | | NASNASTPGPDGVGKRD | | | | GCTGTTCTAG | | |
| | | ISNWDPECETLTQLFNM | | | | TCTGGACCGA | | |
| | | WWFTGVI | | | | GGGTCGGACG | | |
| | | PSRLKKSRTVLLPKSSD | | | | GGGCATTTGA | | |
| | | PGAEMEIGNWRPITIGS | | | | AGGTAGCTGG | | |
| | | MVLRLFTRVI | | | | AATCCTCCGC | | |
| | | NTRLTEACPLHPRQRGF | | | | TGCTGCGAGC | | |
| | | RRSPGCSENLEVLECLL | | | | CTGAGGTCGA | | |
| | | RHSKEKRS | | | | TGGTTAGAGG | | |
| | | QLAVVFVDFAQAFDTVS | | | | TGAAATACTT | | |
| | | HEHMLSVLEQMNVDPHM | | | | GGGAGGAGAC | | |
| | | VNLIREIYT | | | | ACAGCCTCCG | | |
| | | NSCTSVELGRKEGPDIP | | | | GAGAGCCCCT | | |
| | | VRVGVKQGDPLSPLLFN | | | | CCCGGGTGGT | | |
| | | LALDPLIQS | | | | CATCATGGCA | | |
| | | LERTGKGCEAEGHKVTA | | | | ACCGGGTGAA | | |
| | | LAFADDLALVAGSWEGM | | | | ACCTTACGGT | | |
| | | AHNLALV | | | | TTCACTTACG | | |
| | | DEFCLTTGLTVQPKKCH | | | | AAACAGCACC | | |
| | | SFMVRPCRGAFTVNDCP | | | | ATAACAGCGC | | |
| | | PWVLGGK | | | | CGTAATAGCG | | |
| | | ALQLTNIENSIKYLGVK | | | | CACCGGTGTG | | |
| | | VNPWAGIEKPDLTVALD | | | | ACTACTGTCC | | |
| | | RWCKRIGKSL | | | | AGTGCTGATA | | |
| | | LKPSQKVYILNQFAIPR | | | | TTCTCATCTG | | |
| | | LFYLADHGGAGDVMLQN | | | | GAGAATACAA | | |
| | | LDGTIRKAV | | | | CACGGGTAAT | | |
| | | KKWLHLPPSTCNGLLYA | | | | GGCAGAGTAT | | |
| | | RNCNGGLGICKLTRHIP | | | | TCAAAACCCA | | |
| | | SMQARRMF | | | | AATGTTTACG | | |
| | | RLANSSDPLMKAMMRGS | | | | ATCGACCAAC | | |
| | | RVEQKFKKAWMRAGGEE | | | | GGAGTCGTTC | | |
| | | SALPRV | | | | CCTTGCATCT | | |
| | | FGANQYQEGEEVANDLV | | | | AGGCCGGACC | | |
| | | PRCPMPSDWRLEEFQHW | | | | CGAAACTGCC | | |
| | | MGLPIQ | | | | GTAATTGCCC | | |
| | | GVGIAGFFRNRVANGWL | | | | GTCCCCAAGG | | |
| | | RKPAGFKERHYIAALQL | | | | TAGCCTCTTA | | |
| | | RACVYPTL | | | | GAAAACCGAA | | |
| | | EFQQRGRSKAGAACRRC | | | | GCCCGGTCGG | | |
| | | SSRLESSSHILGKCPAV | | | | GGCGGTGGTT | | |
| | | QGARIRRH | | | | GCGGCGGCGC | | |
| | | NKICDLLKAEAETRGWE | | | | TGCGGGGGCC | | |
| | | VRREWAFRTPAGELRRL | | | | TGCTGCTCGG | | |
| | | DLVLILGDE | | | | GCGGCGTCGG | | |
| | | ALVIDVTVRYEFAPDTL | | | | TGTGCCGCGG | | |
| | | QNAGKDKVSYYGPHKEA | | | | TGGTTGCGGT | | |
| | | IARELGVRR | | | | GGTGCGGCGG | | |
| | | VDIHGFPLGARGLWLAS | | | | GGATCTCGGT | | |
| | | NSKVLELMGLSRERVKV | | | | CCTTGCGGTG | | |
| | | FSRLLSRR | | | | CCGCTGTGCC | | |
| | | VLLYSIDIMRTFYATLQ | | | | GCCGCGGTCG | | |
| | | | | | | CGTCGGTGGC | | |
| | | | | | | GCTGGGGTGG | | |
| | | | | | | TGGCCCGAGT | | |
| | | | | | | GGCGTCGGCG | | |
| | | | | | | TGCCACTGCC | | |
| | | | | | | CATAGTCGCC | | |
| | | | | | | CGCGGGGGCG | | |
| | | | | | | ACCGATCTGG | | |
| | | | | | | AGGGGCGAGG | | |
| | | | | | | GGGCTCGCGG | | |
| | | | | | | GACTTTAACG | | |
| | | | | | | AGAAACGGAA | | |
| | | | | | | CGCAACTTCT | | |
| | | | | | | CGCATCGCTC | | |
| | | | | | | CCGGGACTTT | | |
| | | | | | | CCCCCCTCGT | | |
| | | | | | | TCAGCCGAGG | | |
| | | | | | | GATGCCAAAA | | |
| | | | | | | GGCATGAAAG | | |
| | | | | | | GTAAGTACCA | | |
| | | | | | | TACCGGTCCG | | |
| | | | | | | CAAAACTCTC | | |
| | | | | | | TTCTGACTCG | | |
| | | | | | | GTTCTCTGTT | | |
| | | | | | | GGTTTTCTAG | | |
| | | | | | | AGTAACAACG | | |
| | | | | | | AGGTGGAGGA | | |
| | | | | | | GAGGGACATG | | |
| | | | | | | GCAGGGACTC | | |
| | | | | | | CCATTCGTGC | | |
| | | | | | | CAGCGGGTGG | | |
| | | | | | | GGACAGATCG | | |
| | | | | | | AAGGAACGGT | | |
| | | | | | | TCGAGGGCGT | | |
| | | | | | | AACAGACGAG | | |
| | | | | | | AGGGAATCCG | | |
| | | | | | | GTCACACATT | | |
| | | | | | | GATGCCATGC | | |
| | | | | | | CTAAATAGGC | | |
| | | | | | | GAGGTTTGTA | | |
| | | | | | | TTTCTACTTT | | |
| | | | | | | GTGGGTTCAG | | |
| | | | | | | TATAGTCGGA | | |
| | | | | | | GCATATGGTC | | |
| | | | | | | GGTTGTCCCG | | |
| | | | | | | TTGTTTTCAC | | |
| | | | | | | GGCGGGCAAG | | |
| | | | | | | CGACTATCAT | | |
| | | | | | | GATAAAGTAG | | |
| | | | | | | AATGGGAGAC | | |
| | | | | | | GGGCTCCCTG | | |
| | | | | | | ACAAACCCGG | | |
| | | | | | | AAAGGCGCCC | | |
| | | | | | | CCCCGTGGTT | | |
| | | | | | | CGTAGCAGCT | | |
| | | | | | | GACGGATCAC | | |
| | | | | | | GCTCGAAGAA | | |
| | | | | | | AAATGAGTGA | | |
| | | | | | | GAGGGGACGC | | |
| | | | | | | CGCAACCAC | | |
| Oryzias | R201 | MGTDTVYVGQDYPSGLS | 33490 | CGCACAGGGG | 33500 | GGGGGACAGC | 33512 | LC349444.1 |
| latipes | | KRVPARLVAGPMLRERS | | ACACAGAGCC | | TGGGAGTCTC | | |
| | | CHAHVFR | | TGCCCAAGTA | | GGCATGATTA | | |
| | | AGHMWNWRTSLPSGRWD | | CCGCTCCCGA | | CAAATCTTGC | | |
| | | QPALEKSRVLTRSVATA | | GGGAGCGGGA | | GCTGCACTCG | | |
| | | TDPEITS | | AACGGGGGGG | | GATGTCGTCC | | |
| | | YPGKSVSTSTQVQEEDW | | TGACTATCCC | | CCGTGACGGA | | |
| | | CSRESGWISPGLAPEEP | | CTGGGGTCCG | | CACATTAATC | | |
| | | SWSEITA | | GCGAGAGCGC | | CGGAAAGCGA | | |
| | | SMVATMRVATEEVVLEP | | TGGTCTACGG | | GTGGTGACTC | | |
| | | QPEQVVTILPEHGRNVP | | ACCAGGGGTG | | GCCTCAAG | | |
| | | PGLAEQDT | | GCTGTGGGCA | | | | |
| | | ASPIEVSVLLPDLAENC | | GGCTGCTCCT | | | | |
| | | PLCGVPSGGLRLLGKHF | | CAGGCCAGTT | | | | |
| | | AVRHAGVPV | | GATTAGTTAC | | | | |
| | | TYECRKCAWRSPNSHSI | | GCATGGGCTG | | | | |
| | | SCHVPKCRGRARMPSGD | | TACCTCCACG | | | | |
| | | PGIACDL | | TGGTCCCGCT | | | | |
| | | CEARFATEVGVAQHKRH | | GGTAACGACT | | | | |
| | | VHPVEWNKVRLERRGAR | | TGTCGGCTAA | | | | |
| | | GGGIKAT | | ATCAGCCCGC | | | | |
| | | KLWSVAEVETLIRLIRE | | CCACCATCTG | | | | |
| | | HGDSGATYQLIADELGR | | GGATATGGTT | | | | |
| | | GKTAEQVRS | | GACCGTCTAA | | | | |
| | | KKRLLRIDTASNSPDDA | | CCCCAGTACT | | | | |
| | | EVEEERLESLAVRSSSR | | CAGGTCACAA | | | | |
| | | SPPSLVATR | | ACAAA | | | | |
| | | VREAVARGESEGGEEIR | | | | | | |
| | | AIAALIRDVDQNPCLIE | | | | | | |
| | | TSASDIISKLG | | | | | | |
| | | RRVDGPKRPRPVVREQT | | | | | | |
| | | QEKGWVRRLARRKREYR | | | | | | |
| | | EAQYLYS | | | | | | |
| | | RDQARLAAQILDGAASQ | | | | | | |
| | | ECALPVDQVYGAFREKW | | | | | | |
| | | ETVGQFH | | | | | | |
| | | GLGEFRTGARADNWEFY | | | | | | |
| | | SPILAAEVKENLMRMAN | | | | | | |
| | | GTAPGPD | | | | | | |
| | | RISKKALLDWDPRGEQL | | | | | | |
| | | ARLYTTWLIGGVIPRVF | | | | | | |
| | | KECRTKLLP | | | | | | |
| | | KSSDPVELQDIGGWRPV | | | | | | |
| | | TIGSMVTRLFSRILTMR | | | | | | |
| | | LTRACPINP | | | | | | |
| | | RQRGFLASSSGCAENLL | | | | | | |
| | | IFDEIVRRSRRDGGPLA | | | | | | |
| | | VVFVDFARA | | | | | | |
| | | FDSISHEHILCVLEEGG | | | | | | |
| | | LDRHVIGLIRNSYVDCV | | | | | | |
| | | TRVGCVEGM | | | | | | |
| | | TPPIQMKVGVKQGDPMS | | | | | | |
| | | PLLFNLAMDPLIHKLET | | | | | | |
| | | AGTGLKWG | | | | | | |
| | | DLSIATLAFADDLVLVS | | | | | | |
| | | DSEEGMGRSLGILEKFC | | | | | | |
| | | QLTGLRVQP | | | | | | |
| | | RKCHGFFMDKGVVNGCG | | | | | | |
| | | TWEICGSPIHMIPPGES | | | | | | |
| | | VRYLGVQV | | | | | | |
| | | GPGRGVMEPDLIPTVHT | | | | | | |
| | | WIERISEAPLKPSQRMR | | | | | | |
| | | VLNSFALPR | | | | | | |
| | | IIYQADLGKVTVTKLAQ | | | | | | |
| | | IDGIVRKAVKKWLHLSP | | | | | | |
| | | STCNGLLYSR | | | | | | |
| | | NRDGGLGLLKLERLIPS | | | | | | |
| | | VRTKRIYRMSRSPDIWT | | | | | | |
| | | RRMTSHSVS | | | | | | |
| | | KSDWEMLWVQAGGERGS | | | | | | |
| | | APVMGAVEAAPTDVERS | | | | | | |
| | | PDYPDW | | | | | | |
| | | RREENLAWSALRVQGVG | | | | | | |
| | | ADQFRGDRTSSSWIAEP | | | | | | |
| | | ASVGFAQ | | | | | | |
| | | RHWLAALALRAGVYPTR | | | | | | |
| | | EFLARGKEKSGAACRRC | | | | | | |
| | | PARLESCS | | | | | | |
| | | HILGQCPFVQANRIARH | | | | | | |
| | | NKVCVLLATEAERFGWT | | | | | | |
| | | VIREFRLED | | | | | | |
| | | AAGGLKIPDLVCKKADT | | | | | | |
| | | VLIVDVTVRYEMDGETL | | | | | | |
| | | KRAASEKVK | | | | | | |
| | | HYLPVGQQITDKVGGRC | | | | | | |
| | | FKVMGFPVGARGKWPAS | | | | | | |
| | | NNTVLAE | | | | | | |
| | | LGVPAGRMRTFARLVSR | | | | | | |
| | | RTLLYSLDILRDFMREP | | | | | | |
| | | AGRGTRVA | | | | | | |
| | | LIPAATGAAN | | | | | | |

### Example 8. Examination of R2Tg Activity

As R2Tg had the highest insertion activity, we continued to explore the programmability of this R2 system. The characterization of R2Tg enzymatic activities and payload flexibility at the 28S locus and a reprogrammed target in human cells were assessed (FIG. 30A-C, 32, 54A-C, 55A-C). We tested R2Tg for heterologous activity at the endogenous 28S locus by designing an EGFP payload flanked by the cognate R2Tg 5′ and 3′ UTRs and 28S homology arms. Co-transfection of this engineered payload together with wild-type R2Tg resulted in EGFP insertion into endogenous 28S loci, as determined by left and right PCR junctional analysis (FIG. 30A). To verify dependence on the retrotransposition mechanism, we introduced inactivating mutations in the RLE endonuclease domain (R2Tg_{D1274A}) and ZF domain (R2Tg_{ZF2mut}), and found these mutations ablated insertion activity (FIG. 30B). Alternatively, mutations introduced at catalytic residues in the RT domain (R2Tg_{D877A,D878A,D884A}) significantly reduced, but did not eliminate, insertion activity (FIG. 30B), which we confirmed by quantifying insertion events using next generation sequencing of targeting amplicons (NGS) (FIG. 30C). We validated these findings on inactivating mutations with our plasmid reporter assay by both luciferase production and editing by NGS (FIG. 30D and FIG. 31A-B). Based on NGS reads and gel electrophoresis readouts, all R2Tg insertions were found to be full length (FIG. 30), in contrast to observed partial insertions with R2Ol (Su et al., 2019 RNA. 25, 1432-1438). However, R2Tg integration was accompanied by indels at the 28S target, consistent with the previously observed non-templated addition of deoxycytidines by the RT domain (Bibillo, et al., 2004. Journal of Biological Chemistry. 279, pp. 14945-14953.) (FIG. 31C). To finely profile boundaries of functional domains, we tested N- and C-terminal truncations, finding that no C-terminal truncations were tolerated, likely due to loss of the RLE domain, whereas N-terminal truncations were tolerated up to the ZF motifs (FIG. 32). These results show the necessity of the ZF and RLE domains for activity and demonstrate that the N-terminal domain upstream of the ZF motifs does not critically contribute to the insertion process.

Having determined that the R2TgZF2mut mutant ablated integration, we speculated that supplementing additional DNA binding or nicking activity could rescue R2 integration activity at the 28S target site. We mutated Cas9 from Streptococcus pyogenes to generate either nickase (SpCas9H840A) or dead (SpCas9D10A,H840A) variants and fused these Cas9 variants via an XTEN linker to the N-terminus of R2TgΔ1-184,ZF2mut, which contains both a truncation that retains activity (Δ1-184) and the inactivating ZF2 domain mutation (ZF2mut). We then designed Cas9 guides against the 28S target region and coupled these with the R2Tg variants (FIG. 33A). We found that both single guides and paired guides around the target site were able to recruit with SpCas9H840A-R2TgZF2mut and restore integration at the locus, up to 72% of that of WT R2Tg (FIG. 33B-C). However, fusions of SpCas9D10A,H840A with the ZF2 mutant failed to restore activity, implying that the ZF2 binding was necessary for successful nicking (FIG. 34A). We also observed that SpCas9H840A fusion to the RLE deficient mutant R2TgD1274A failed to rescue insertion activity, suggesting involvement of the native nicking of the RLE domain in insertion process, perhaps for second strand nicking or initiation of second strand synthesis (FIG. 34B). These observations held over a larger panel of mutations in the RT and RLE domains as well (FIG. 34C). Therefore, the insertion process involving the RLE and RT domains can be rescued when combined with SpCas9H840A nickase, suggesting a possible route for evolutionary retargeting dependent on ZF evolution or reengineering via Cas9 supplementation.

### Example 9. Identification of Factors Affecting Integration Dynamics

Given that the homology of the RNA template is a strong determinant of the target site, we probed the necessary homology for integration. We tested iterative truncations of either the 5′ or 3′ homology regions (FIG. 35A), finding that, while the 5′ region was sensitive to truncation and required a minimum homology between 20 and 40 nt, the 3′ region was robust to truncations, allowing efficient integration even in the absence of homology (FIG. 35B). This leniency may be due in part to promiscuous priming of the R2 systems, suggesting an avenue for evolving preferences to new loci via asymmetric acquisition of homology that would be compatible with Class 1 insertions observed in our computational exploration.

The malleable constraints of RNA cargo homology, especially at the 3′ end, prompted us to test cargo components. We next tested whether priming could occur internally to cargo, which would allow for successful integration after swapping the UTR and homology regions. Successful insertion from internal homology allows for scarless integration, with significant gene editing applications (FIG. 35C). We evaluated a panel of cargo permutations (FIG. 36A), swapping or duplicating homology elements to investigate whether internal homology could allow for template insertion. Moving homology internal to the UTR resulted in successful scarless insertions (FIG. 36C and FIG. 36B), as confirmed by sanger sequencing, suggesting flexible template priming. Traces of payloads with homology external to the 5′ UTR, had a loss of phasing at the 5′ junction due to multiple populations, and next generation sequencing confirmed these were due to non-templated addition of nucleotides, similar to what we observed at the wildtype 28S locus (FIG. 37A-B). Analysis of cross-junctional PCR products of insertion products with cargos having internal homology showed a reduction of size corresponding to a complete absence of the UTR region (FIG. 37C, FIGS. 38A-D, and 60), confirming scarless insertion, the flexibility of the retrotransposon cargo architecture, and the efficiency of scarless integration especially with cargo 6 (FIG. 38C-D). These results highlight that priming off the template is very flexible, implying a very direct path for an expressed retrotransposon to acquire new priming sequences by landing in new areas via promiscuous priming of novel target sites or acquiring nearby targets and supporting both the Class 1 and Class 2 insertion mechanism.

While permutations of cargo components and complete removal of the 3′ UTR were tolerated, deletion of the 5′ UTR region resulted in significantly lower integration rates (FIG. 38A); as integration was not completely eliminated, this suggests that some element of the 28S homology region is still recognized by R2Tg for integration. All tested payloads also had some amount of residual indel formation (FIG. 37B, FIG. 38A-B). Overall, moving homology regions internal to the UTRs could produce scarless insertion at both 5′ and 3′ junctions while still maintaining efficient integration, especially with cargo 9 (FIG. 38A). These modifications also show that scar formation of R2Tg due to external homology is not a necessary component for the system to function and suggested the feasibility of acquiring new sites via similarity to internal regions.

### Example 10. Programming of R2Tg for Integration at Specific Loci

We next programmed the R2Tg system to integrate at different loci by swapping target homologies (FIG. 39A). We designed scarless insertion payloads with homology arms to either AAVS1 or NOLC1 loci and co-transfected these payloads with R2Tg in HEK293FT cells, finding targeted insertion at NOLC1 at ˜0.5%, which depended on the RLE catalytic residues of R2Tg, and no detectable insertion at AAVS1 (FIG. 39B). To improve efficiencies of retargeting R2Tg at both loci, we tested whether SpCas9H840A could improve insertion through additional nicking activity. We designed a pair of guide RNAs to introduce nicks on the bottom and top strands of the NOLC1 locus or a single guide RNA to introduce a nick at the AAVS1 locus. We co-delivered these along with a cargo carrying transgene payloads, 5′ and 3′ R2Tg UTRs, and homology arms directed around the nicking site of 100 or 50 nt for AAVS1 and NOLC1 respectively. We found that SpCas9H840A-R2Tg fusion had increased efficiency at both NOLC1 (˜3%) and AAVS1 (˜0.5%) (FIG. 39B), showing that SpCas9H840A could significantly improve R2Tg insertion efficiency.

To find optimal payloads for efficient insertion at new loci, we designed a panel of payloads following integration guidelines that were effective at the 28S locus (FIG. 39C). Across designs, including internally located homology arms, deletion of the 3′ UTR, and truncation of the 5′ UTR, we progressively observed increased integration at the AAVS1 locus up to 6% (FIG. 39D). Importantly, addition of the 5′ 28S homology arm upstream of the 5′ UTR in payloads 2, 6, and 7 substantially improved integration activity. Payloads 4-7 with homology arms directly flanking the insert had scarless insertion at the AAVS1 locus, with minimal indel formation and perfect insertions representing more than 99% of the editing activity. The most truncated 5′ UTR sequence had the highest editing, suggesting that R2Tg recognition of the payload only required a small sequence region. As was expected from 28S payload characterization, the 3′ UTR was also dispensable for AAVS1 insertion. We also examined whether insertion activity at the endogenous AAVS1 locus could be optimized via better SpCas9H840A fusions to different R2Tg truncations, again finding that C-terminal truncations were not tolerated, whereas the 1-184 residue truncation of R2Tg had maximum activity compared to other truncations while offering a more compact version of the SpCas9H840A-R2Tg fusion (FIG. 40A-B). To test the dependence of these SpCas9H840A-R2Tg fusions on the binding and nicking activities of SpCas9H840A independently, we compared the SpCas9H840A-R2Tg fusion to the dead SpCas9D10A,H840A-R2Tg fusion, as well as SpCas9H840A and R2Tg delivered in trans as separate proteins. We found that, while the SpCas9D10A,H840A-R2Tg had no insertion activity, co-delivery of SpCas9H840A and R2Tg was sufficient to efficiently insert the payload at both NOLC1 and AAVS1 loci, showing that the nicking activity, but not binding activity, of SpCas9H840A was enhancing insertion efficiency (FIG. 40C).

As some R2 retrotransposons have been proposed to function as a homodimer upon binding their cognate RNA templates (Yang et al., 1998. Mol. Cell. Biol. 18, 3455-3465), we were motivated to explore whether dual guides on opposing DNA strands might emulate dual nicking and recruitment of R2Tg and stimulate more efficient integration. Comparing single and dual guides, we found that certain paired guides achieved up to 15% integration with minimal indels generated and near perfect integration >99% using payloads with 100 nt of homology (FIG. 41A). Specific combinations of paired guides had low levels of integration with SpCas9H840A alone, indicating some contribution from HDR mediated insertion of the payload off the DNA vector, and this effect was less prominent with single guides. Interestingly, top strand nicking guides, such as guide A4, could promote insertion, suggesting that the RLE domain of the R2Tg protein could initiate bottom strand nicking at the AAVS1 target (FIG. 41A). To reduce HDR background, we tested payloads with homology arms reduced from 100 nt to 50 nt, and found that these designs maintained insertion while blunting HDR byproducts (FIG. 41B). Surprisingly, while integration with SpCas9H840A-R2Tg had minimal indel formation, SpCas9H840A alone generated substantially more indels at the WT locus, indicating competition between complete integration and continued nicking and indel formation (FIG. 41A).

### Example 11. Further Development and Integration of Re-Targeted Retrotransposons

We next determined whether diverse non-LTR retrotransposons could be repurposed for integration in cells despite failing at the 28S locus. To compensate for potentially ineffective binding or cleavage at the 28S locus in mammalian cells, we fused a panel of 11 additional retrotransposon candidates to SpCas9H840A and tested them for additional guided insertion improvements at the 28S locus. We found that several of the retrotransposons, including many without activity at 28S target, had significant increases in 28S insertion when paired with targeting guides (FIG. 42A), including R2Bm, R2Ci, HeroDr, R10Mbr, R2Oi, and R2Tsp, whereas R2Tg and R2Mes did not have further increases, likely due to their already high natural integration.

We modified corresponding payloads for scarless insertion by rearranging homology regions internal to UTRs, and reprogrammed homology regions and SpCas9 guides to target payloads to the AAVS1 locus (FIG. 58B). We found that our framework for retrotransposon retargeting generalized, with 6 out of 12 orthologs tested efficiently reprogrammed (FIG. 42B-D) showing efficient reprogramming, with minimal indel formation accompanying the insertion activity (FIGS. 58B and 68). R2Toc had a high efficiency of reprogramming with strongly reduced background 28S insertion (FIG. 29A).

### Example 12. Site-specific Target-Primed Insertion Via Targeted CRISPR Homing of Retroelements (STITCHR)

After developing our SpCas9H840A-R2Toc-based insertion system, which we refer to as Site-specific Target-primed Insertion via Targeted CRISPR Homing of Retroelements (STITCHR), we explored multiple applications for STITCHR-based programmable gene insertion in mammalian cells. To generalize STITCHR reprogramming to other loci beyond AAVS1, we targeted the NOLC1 and SERPINA1 loci with panels of single and dual guides, finding that dual guides integration efficiencies up to 13% and 10% insertion at NOLC1 and SERPINA1, respectively (FIG. 43A-D). While single guides had measurable integration activity, dual guides tended to better promote integration. Assaying the fidelity of insertion, we found low indel formation at these sites, indicating that integration was with high fidelity and that integration by SpCas9H840A-R2Toc outcompeted indel formation, as indels were higher with SpCas9H840A alone (FIG. 43A, 43D). Moreover, integration was scarless, as would be expected from our payloads with internal homology regions. While many dual guide combinations and single guides enabled STITCHR gene insertion, minimal editing was observed with SpCas9H840A alone. Interestingly, single top strand nicking guides, in addition to bottom strand guides, could stimulate STITCHR insertion, suggesting R2Toc RLE domain participation in bottom strand nicking (FIG. 43A, 43C). Furthermore, we found that elimination of payload homology at NOLC1 or substitution for non-homologous sequences ablated editing (FIG. 44A). Conversely, increasing payload homology did not substantially increase integration efficiency, but led to higher background due to HDR (FIG. 44B). We also found that for high background loci, reducing payload homology could support gene integration with reduced HDR background, as observed at the AAVS1 and SERPINA1 loci with SpCas9H840A-R2Toc where we could achieve 8% gene integration with no background from HDR (FIG. 44C-D). Interestingly, we observed that R2Toc, like R2Tg, was also capable of programmable insertion without the assistance of Cas9 via the payload homology as the non-targeting guide conditions had 2% NOLC1, 1.3% AAVS1, and 0.35% SERPINA1 insertion (FIG. 43A-B, FIG. 44C, FIG. 71).

To take advantage of scarless genome insertion with R2Toc, we investigated whether we could place an EGFP tag in-frame to a protein target. We chose NOLC1 due to its distinct nuclear organization and designed our template in the reverse direction to prevent constitutive expression of the EGFP off the template cargo (FIG. 45A). We found that STITCHR-mediated GFP insertion led to NOLC1 tagging, as verified by confocal imaging and corresponding colocalization with immunofluorescence staining (FIG. 45B). We then explored additional payload flexibility of the STITCHR system at the AAVS1 locus, using a panel of cargo sequences of different lengths. Evaluating various therapeutically relevant genes, including BTK, CEP290, HBB, HEXA, OTC, and PAH, we found insertion efficiencies of 10-20% at the AAVS1 locus with minimal insertion using SpCas9H840A alone (FIG. 45C-D, FIG. 74). These payloads varied in size between 0.7-7.7 kb, showing that STITCHR mediated insertion can insert a wide range of insert sizes.

Multiple types of genomic edits by STITCHR, including single base edits, small insertions, and a range of large payload insertions are enabled by the flexible nature of the retrotransposon insertion pathway (FIG. 59A-B, FIG. 61, FIG. 69A-C, FIG. 70A-C). We tested a panel of edits at the NOLC1 locus, spanning single base changes (transitions and transversions), small insertions (1-10 bp), therapeutic cargos, and large gene insertions. STITCHR could effectively install these diverse edit types (FIG. 59C), including therapeutically relevant genes of different sizes, such as BTK, CEP290, HBB, HEXA, OTC, and PAH, and synthetic sequences up to 10.9 kb. STITCHR also inserted these therapeutic genes at AAVS1 (FIG. 45D). For cargos carrying small edits, we found both that extending the transcript beyond the homology arms further improved editing, presumably due to stabilization of the RNA transcript or better expression from the Pol II promoter (FIG. 59C), and U6 promoters could effectively produce smaller templates and genomic edits (FIG. 72D). To combine STITCHR-mediated insertion with deletion, we tested simultaneous replacement of genomic regions by separating the homology target sites by 50-150 bp (FIG. 60A), which we refer to as STITCHR-replace. We found that an EGFP payload could be simultaneously inserted while replacing 50-150 bp of genomic sequence with 6-7% integration efficiency (FIG. 60B and FIG. 73).

To test STITCHR activity in a non-dividing context, we inhibited HDR using the cell cycling inhibitor aphidicolin, which traps cells at the G1/S phase transition and inhibits HDR activity. We found that STITCHR integration of an EGFP cargo at the NOLC1 locus was not inhibited by increasing concentrations of aphidicolin and led to increases in efficiency at intermediate aphidicolin concentrations. In contrast, HDR integration by SpCas9 nuclease at the EMX1 locus was inhibited by up to 94% by aphidicolin (FIG. 46A-B), and residual NOLC1 HDR insertion observed with SpCas9H840A alone was eliminated at all tested aphidicolin concentrations, further demonstrating a retrotransposition and HDR-independent based mechanism for insertion (FIG. 46B).

To extend STITCHR to multiplexed editing without reliance on cell division, we investigated whether STITCHR could mediate multiplexed integration at two different sites in the genome. We simultaneously delivered guide RNAs and cargos targeting the AAVS1 and NOLC1 loci for Gluc and EGFP insertion, respectively, finding that multiplexed insertion was possible with 12% and 6% integration at the AAVS1 and NOLC1 loci, respectively (FIG. 47A-B).

We also examined STITCHR in the context of a concurrent insertion/deletion approach. We compared a SpCas9^{H840A}-R2Toc using a single fixed guide RNA (N4, see table 7) to target the NOLC1 locus to that of the non-targeting, SpCas9^{H840A} alone. An EGFP insert was used as a payload. When homology arms on the payload template were separated by 0 bp, 50 bp, 100 bp, or 150 bp, we were successfully able to delete the genomic target while concurrently inserting the EGFP payload into the NOLC1 locus (FIG. 49). Exemplary sequences for the 50 bp deletion, 100 bp deletion, and 150 bp deletion, as well as the Guide sequence used for this experiment are found in Table 7.

TABLE 7
EGFP Cargo and Guide Sequences for Concurrent
Insertion/Deletion

| | | SEQ | | SEQ |
|---|---|---|---|---|
| | | ID | Guide | ID |
| Cargo | Sequence | NO: | Sequence | NO: |
| −50 | atccttaatattcaatgaagcgcgggtaaacggcgggag | 33513 | GACGCGTAT | 33425 |
| deletion | taactatgactctcttaaggtctagttacaactggTCCT | | TGCCTGGAG | |
| | GAGTCGTGCTGCGTCGACAACGGTAGTGACGCGTATTGC | | GA | |
| | CTGGAGGCCGCCACCATGCCCGCCATGAAGATCGAGTGC | | | |
| | CGCATCACCGGCACCCTGAACGGCGTGGAGTTCGAGCTG | | | |
| | GTGGGCGGCGGAGAGGGCACCCCCGAGCAGGGCCGCATG | | | |
| | ACCAACAAGATGAAGAGCACCAAAGGCGCCCTGACCTTC | | | |
| | AGCCCCTACCTGCTGAGCCACGTGATGGGCTACGGCTTC | | | |
| | TACCACTTCGGCACCTACCCCAGCGGCTACGAGAACCCC | | | |
| | TTCCTGCACGCCATCAACAACGGCGGCTACACCAACACC | | | |
| | CGCATCGAGAAGTACGAGGACGGCGGCGTGCTGCACGTG | | | |
| | AGCTTCAGCTACCGCTACGAGGCCGGCCGCGTGATCGGC | | | |
| | GACTTCAAGGTGGTGGGCACCGGCTTCCCCGAGGACAGC | | | |
| | GTGATCTTCACCGACAAGATCATCCGCAGCAACGCCACC | | | |
| | GTGGAGCACCTGCACCCCATGGGCGATAACGTGCTGGTG | | | |
| | GGCAGCTTCGCCCGCACCTTCAGCCTGCGCGACGGCGGC | | | |
| | TACTACAGCTTCGTGGTGGACAGCCACATGCACTTCAAG | | | |
| | AGCGCCATCCACCCCAGCATCCTGCAGAACGGGGGCCCC | | | |
| | ATGTTCGCCTTCCGCCGCGTGGAGGAGCTGCACAGCAAC | | | |
| | ACCGAGCTGGGCATCGTGGAGTACCAGCACGCCTTCAAG | | | |
| | ACCCCCATCGCCTTCGCCAGATCTCGAGCTCGAGCTCGG | | | |
| | CTTCCTGCGCGATAACCAACTCTCAGAGGTGGCCAATAA | | | |
| | GTTCG | | | |
| −100 | atccttaatattcaatgaagcgcgggtaaacggcgggag | 33514 | GACGCGTAT | 33425 |
| deletion | taactatgactctcttaaggtctagttacaactggTCCT | | TGCCTGGAG | |
| | GAGTCGTGCTGCGTCGACAACGGTAGTGACGCGTATTGC | | GA | |
| | CTGGAGGCCGCCACCATGCCCGCCATGAAGATCGAGTGC | | | |
| | CGCATCACCGGCACCCTGAACGGCGTGGAGTTCGAGCTG | | | |
| | GTGGGCGGCGGAGAGGGCACCCCCGAGCAGGGCCGCATG | | | |
| | ACCAACAAGATGAAGAGCACCAAAGGCGCCCTGACCTTC | | | |
| | AGCCCCTACCTGCTGAGCCACGTGATGGGCTACGGCTTC | | | |
| | TACCACTTCGGCACCTACCCCAGCGGCTACGAGAACCCC | | | |
| | TTCCTGCACGCCATCAACAACGGCGGCTACACCAACACC | | | |
| | CGCATCGAGAAGTACGAGGACGGCGGCGTGCTGCACGTG | | | |
| | AGCTTCAGCTACCGCTACGAGGCCGGCCGCGTGATCGGC | | | |
| | GACTTCAAGGTGGTGGGCACCGGCTTCCCCGAGGACAGC | | | |
| | GTGATCTTCACCGACAAGATCATCCGCAGCAACGCCACC | | | |
| | GTGGAGCACCTGCACCCCATGGGCGATAACGTGCTGGTG | | | |
| | GGCAGCTTCGCCCGCACCTTCAGCCTGCGCGACGGCGGC | | | |
| | TACTACAGCTTCGTGGTGGACAGCCACATGCACTTCAAG | | | |
| | AGCGCCATCCACCCCAGCATCCTGCAGAACGGGGGCCCC | | | |
| | ATGTTCGCCTTCCGCCGCGTGGAGGAGCTGCACAGCAAC | | | |
| | ACCGAGCTGGGCATCGTGGAGTACCAGCACGCCTTCAAG | | | |
| | ACCCCCATCGCCTTCGCCAGATCTCGAGCTCGACCAAAG | | | |
| | CGACAGGAGCTGTGAGTTCCGGGCTTGGGGCGGGGACCG | | | |
| | GGCTG | | | |
| −150 | atccttaatattcaatgaagcgcgggtaaacggcgggag | 33515 | GACGCGTAT | 33425 |
| deletion | taactatgactctcttaaggtctagttacaactggTCCT | | TGCCTGGAG | |
| | GAGTCGTGCTGCGTCGACAACGGTAGTGACGCGTATTGC | | GA | |
| | CTGGAGGCCGCCACCATGCCCGCCATGAAGATCGAGTGC | | | |
| | CGCATCACCGGCACCCTGAACGGCGTGGAGTTCGAGCTG | | | |
| | GTGGGCGGCGGAGAGGGCACCCCCGAGCAGGGCCGCATG | | | |
| | ACCAACAAGATGAAGAGCACCAAAGGCGCCCTGACCTTC | | | |
| | AGCCCCTACCTGCTGAGCCACGTGATGGGCTACGGCTTC | | | |
| | TACCACTTCGGCACCTACCCCAGCGGCTACGAGAACCCC | | | |
| | TTCCTGCACGCCATCAACAACGGCGGCTACACCAACACC | | | |
| | CGCATCGAGAAGTACGAGGACGGCGGCGTGCTGCACGTG | | | |
| | AGCTTCAGCTACCGCTACGAGGCCGGCCGCGTGATCGGC | | | |
| | GACTTCAAGGTGGTGGGCACCGGCTTCCCCGAGGACAGC | | | |
| | GTGATCTTCACCGACAAGATCATCCGCAGCAACGCCACC | | | |
| | GTGGAGCACCTGCACCCCATGGGCGATAACGTGCTGGTG | | | |
| | GGCAGCTTCGCCCGCACCTTCAGCCTGCGCGACGGCGGC | | | |
| | TACTACAGCTTCGTGGTGGACAGCCACATGCACTTCAAG | | | |
| | AGCGCCATCCACCCCAGCATCCTGCAGAACGGGGGCCCC | | | |
| | ATGTTCGCCTTCCGCCGCGTGGAGGAGCTGCACAGCAAC | | | |
| | ACCGAGCTGGGCATCGTGGAGTACCAGCACGCCTTCAAG | | | |
| | ACCCCCATCGCCTTCGCCAGATCTCGAGCTCGAAGATGA | | | |
| | CCACAAGGCTTCAGGCCCTGACGTGCTTAGGTTTCCAGG | | | |
| | TGGGG | | | |

We further examined the possibility of using STITCHR to create single nucleotide edits and small nucleotide insertions. SpCas9^{H840A}-R2Toc was used with dual guides N4 and N8 (N8 Sequence: GGGAACCACGCGGCGAATGC (SEQ ID NO: 33429)) with a payload of either a GFP insert (FIG. 50A, columns 1-2,) a payload with a 1 bp mismatch to the NOLC1 locus (FIG. 50A, columns 3-8), or a payload with a small nucleotide insert (FIG. 50A, columns 9-14). When compared to the non-targeting SpCas9^{H840A,} the SpCas9^{H840A}-R2Toc system was able to make single base pair edits, as well as small nucleotide inserts (1-50 bp). We also examined the effect of the promoter in driving the STITCHR cargo (FIG. 50B). Use of the CAG promoter to express the cargo resulted in slightly higher editing levels, potentially due to higher expression of the template RNA sequence. Sequences used in these experiments are found at table 8.

TABLE 8
Cargo and Guide Sequences for Concurrent Single Nucleotide
Edits and Small Nucleotide Inserts

| Cargo | Sequence |
|---|---|
| 1bp | attcaatgaagcgcgggtaaacggcgggagtaactatgactctcttaaggtctagttac |
| mismatch A | aactggTCCTGAGTCGTGCTGCGTCGACAACGGTAGTGACGCGTATTGCCTGGAGGACG |
| | GACGCCGGCATTCGCCGCGTGGTTCCCAGCGACCTGTATCCCCTCGT (SEQ ID NO: |
| | 33516) |
| 1bp | attcaatgaagcgcgggtaaacggcgggagtaactatgactctcttaaggtctagttac |
| mismatch C | TaactggTCCTGAGTCGGCTGCGTCGACAACGGTAGTGACGCGTATTGCCTGGAGGCCG |
| | GACGCCGGCATTCGCCGCGTGGTTCCCAGCGACCTGTATCCCCTCGT (SEQ ID NO: |
| | 33517) |
| 1bp | attcaatgaagcgcgggtaaacggcgggagtaactatgactctcttaaggtctagttac |
| mismatch T | aactggTCCTGAGTCGTGCTGCGTCGACAACGGTAGTGACGCGTATTGCCTGGAGGTCG |
| | GACGCCGGCATTCGCCGCGTGGTTCCCAGCGACCTGTATCCCCTCGT (SEQ ID NO: |
| | 33518) |
| 1bp | attcaatgaagcgcgggtaaacggcgggagtaactatgactctcttaaggtctagttac |
| insert | aactggTCCTGAGTCGTGCTGCGTCGACAACGGTAGTGACGCGTATTGCCTGGAGGAGC |
| | GGACGCCGGCATTCGCCGCGTGGTTCCCAGCGACCTGTATCCCCTCGT (SEQ ID |
| | NO: 33519) |
| 10bp | attcaatgaagcgcgggtaaacggcgggagtaactatgactctcttaaggtctagttac |
| insert | aactggTCCTGAGTCGTGCTGCGTCGACAACGGTAGTGACGCGTATTGCCTGGAGGATG |
| | CCCGCCAGCGGACGCCGGCATTCGCCGCGTGGTTCCCAGCGACCTGTATCCCCTCGT |
| | (SEQ ID NO: 33520) |
| 50bp | attcaatgaagcgcgggtaaacggcgggagtaactatgactctcttaaggtctagttac |
| insert | aactggTCCTGAGTCGTGCTGCGTCGACAACGGTAGTGACGCGTATTGCCTGGAGGATG |
| | CCCGCCATGAAGATCGAGTGCCGCATCACCGGCACCCTGAACGGCGTGCGGACGCCGGC |
| | ATTCGCCGCGTGGTTCCCAGCGACCTGTATCCCCTCGT (SEQ ID NO: 33521) |
| 80bp | attcaatgaagcgcgggtaaacggcgggagtaactatgactctcttaaggtctagttac |
| | aactggTCCTGAGTCGTGCTGCGTCGACAACGGTAGTGACGCGTATTGCCTGGAGGATG |
| | CCCGCCATGAAGATCGAGTGCCGCATCACCGGCACCCTGAACGGCGTGGAGTTCGAGCT |
| | GGTGGGCGGCGGAGAGGGGCGGACGCCGGCATTCGCCGCGTGGT (SEQ ID NO: |
| | 33522) |

### Example 13. Trans Delivery of Nuclease Activity

We next tested whether the nuclease activity of the genome editing system had to be provided in cis or if it could be provided in trans. An EGFP payload (with 50 nt homology arms) was used in conjunction with a SpCas9^{H840A}-R2Toc, in which Cas9 is fused to the R2Toc element, targeting the NOLC1 locus with dual N4 and N8 guides, and was compared to the non-targeting SpCas9^{H840A}. In addition, the nuclease activity conferred by SpCas9^{H840A} was also examined with separate (trans) expression of R2Toc (FIG. 51, columns 5-6). This non-linked SpCas9^{H840A} and R2Toc exhibited a payload insertion level similar to that of the fused system, SpCas9^{H840A}-R2Toc. When the nuclease activity was not supplemented with the non-LTR site specific retrotransposon element, little payload insertion was observed.

### Example 14. Methods of the Examples

### Mammalian Cell Culture

HEK293FT cells (ATCC) were cultured in Dulbecco's Modified Eagle Medium with 4.5 g/l glucose, sodium pyruvate, GlutaMAX (Thermo Fisher Scientific) and supplemented with 10% (v/v) fetal bovine serum (FBS) and 1× penicillin-streptomycin (Thermo Fisher Scientific). Cells were maintained below confluency at 37° C. and 5% CO,

### Cell Transfection, Genomic DNA Extraction and Purification

Cells were transfected in 96 well poly-D-Lysine plates (Corning) 16-24 h after plating at a confluency of 70% using Lipofectamine 3000 according to the manufacturer's protocol. In brief, 50 ng R2-expressing plasmid, 50 ng cargo plasmid, 50 ng reporter plasmid (optional) and 30 ng of sgRNA-expressing plasmids were transfected. 72 h post transfection, genomic DNA was isolated by removing media and adding 50 μl QuickExtract (Lucigen) per well. After a 5 min incubation at room temperature, the lysate was transferred to a 96 well PCR plate and incubated at 65° C. for 15 min, 68° C. for 15 min, and 98° C. for 10 min and used as input for targeted deep sequencing. Lysates were further purified using AMPure magnetic beads (Beckman Coulter) according to the manufacturer's protocol and eluted in 25 μL water, if used as input for ddPCR or NGS-based assays.

Editing Quantification by Next Generation Sequencing or ddCPR

Insertion efficiencies into plasmid and genomic DNA were quantified using a 3-primer assay. Here, a forward primer was combined with two reverse primers, one of which binds in the uninserted DNA and the other in inserted DNA. The forward and two reverse primers in a 2:1:1 ratio were added at a total combined concentration of 0.5 μM for a first round PCR counting 20 cycles. A second round PCR with 12 cycles added barcoded primers for Illumina NGS. The 28S, AAVS1, and SERPINA1 experiments were quantified by 3 primer NGS for total integration and indel rates. For NOLC1, the 3-primer assay was used for analyzing indels associated with integration events and the WT locus. NOLC1 total integration was assayed by digital droplet PCR (ddPCR) as described below.

To quantify NOLC1 integration efficiency by digital droplet PCR, 24 solutions were prepared in a 96-well plate containing 1) 12 μL 2 x ddPCR Supermix for Probes (Bio-Rad) 2) primers for amplification of the integration junction at 250 nM-900 nM, 3) FAM probe for detection of the integration junction amplicon at 250 nM 4) 1.44 μL RPP30 HEX reference mix (Bio-Rad) 5) 0.12 μL FastDigest restriction enzyme for degradation of primer off-targets (Thermo Fisher) and 6) Sample DNA at 1-10 ng/μL. The 20 μL of reaction mix was transferred to a Dg8 Cartridge (Bio-Rad) and loaded into a QX2000 droplet generator (Bio-Rad). 40 μL droplets suspended in ddPCR droplet reader oil were transferred to a new 96-well plate and thermocycled according to manufacturer's specifications. Lastly, the 96-well plate was transferred to a QX200 droplet reader (Bio-Rad) and the generated data were analyzed using Quantasoft Analysis Pro to quantify DNA editing.

### Example 15. Cas9-Assisted Retrotransposon Insertion

SpCas9H840A has the potential to improve insertion through recruitment and supplementation of nicking activity (FIG. 56A). A pair of guide RNAs was designed to introduce nicks on the bottom and top strands of NOLC1 and co-delivered these guides with a cargo carrying transgene payloads, a 5′ R2Tg UTR, and internal 50 nt homology arms placed around the nicking site at the NOLC1 locus. SpCas9H840A-R2Tg fusion was found to have increased efficiency at NOLC1 (˜0.6%) (FIG. 56B) in a guide and RLE-dependent fashion, demonstrating that SpCas9H840A can significantly improve R2Tg insertion efficiency.

A panel of payloads was designed to optimize payload design for efficient insertion at retargeted loci. The panel was designed to target the NOLC1 locus to expand upon our initial findings from R2Tg natural insertion at the 28S locus (FIG. 56C). Payloads were designed with varying 5′ UTR sequences by panning 65 nt windows of the annotated 5′ UTR, including regions upstream containing the 5′ 28S homology region to navigate around a potentially relevant HDV-like cleavage site occurring in said region in R2Bm and R2Tg 5,29. Windows overlapping the distal 5′ UTR region and 28S homology region upstream of a 5′ target homology and payload sequence, either with or without a 3′ UTR region, were found to be necessary and sufficient for reprogrammed insertion at NOLC1 (FIG. 56C). A truncated 28S-5′ UTR sequence improved insertion efficiency over the complete 5′ UTR and retained significant secondary structure, indicative of potential conserved function (FIG. 66A). Retargeting at the AAVS1 locus followed similar rules, requiring the upstream 28S and a minimal 15 nt 5′ UTR for efficient gene integration quantified using a validated sequencing assay (FIG. 66B-C, FIG. 75). These results shows that the 28S sequence together with a truncated 5′ UTR sequence, can function as a bona fide 5′ UTR, and that the entire R2Tg 3′UTR can be dispensable for retargeted insertion.

Additionally, insertion activity was tested at the endogenous AAVS1 locus using SpCas9H840A-R2Tg fusion proteins with different R2Tg protein truncations. C-terminal truncations were found to be not tolerated, whereas the 1-184 residue N-terminal truncation of R2Tg retained activity while offering a more compact version of the SpCas9H840A-R2Tg fusion (FIG. 67A-B). To probe the mechanism of retargeted insertion at the NOLC1 and AAVS1 loci by the SpCas9H840A-R2Tg truncation fusion, insertion with the mutagenized variation of the RT domain was tested, confirming that integration was dependent on the RT activities of the retrotransposon and consistent with a TPRT based mechanism for programmable integration (FIG. 58A and FIG. 67C).

1. A genome editing system comprising:
i) an R2 element enzyme; and
ii) a payload RNA,
wherein the payload RNA comprises an insertion template,
comprising a nucleic acid sequence for insertion into a genome, and
wherein the R2 element enzyme comprises a reverse transcriptase domain and a nickase domain.

2. The genome editing system of claim 1, wherein the R2 element enzyme further comprises a targeting domain.

3. (canceled)

4. (canceled)

5. (canceled)

6. (canceled)

7. The genome editing system of claim 1, wherein the R2 element enzyme is modified by an N-terminal or C-terminal truncation.

8. The genome editing system of claim 1, wherein the R2 element enzyme comprises a linker.

9. (canceled)

10. (canceled)

11. The genome editing system of claim 1, wherein the genome editing system targets a genomic locus other than a 28S rRNA locus.

12. (canceled)

13. The genome editing system of claim 1, wherein a non-naturally occurring targeting region is fused to an N-terminus of the R2 element enzyme or fused elsewhere to the R2 element enzyme.

14. (canceled)

15. The genome editing system of claim 1, wherein the R2 element is fused to a Cas9 protein that is fully active, catalytically dead (H840A/D10A for SpCas9), or functions as a nickase (H840A or D10A for SpCas9).

16. The genome editing system of claim 1, wherein the R2 element is fused to a Cas12 protein that is fully active, catalytically dead, or functions as a nickase.

17. The genome editing system of claim 1, further comprising a guide RNA.

18. (canceled)

19. (canceled)

20. The genome editing system of claim 1, wherein the payload RNA further comprises one or more of a 5′ homology region, a 3′ homology region, or a protein binding element.

21. (canceled)

22. (canceled)

23. (canceled)

24. The genome editing system of claim 1, wherein the genome editing system functions in post-mitotic cells.

25. (canceled)

26. The genome editing system of claim 1, wherein the payload RNA further comprises a 5′ untranslated region (UTR), a 3′ UTR, or both a 5′ UTR and a 3′ UTR.

27. (canceled)

28. (canceled)

29. (canceled)

30. (canceled)

31. (canceled)

32. The genome editing system of claim 1, wherein the payload RNA further comprises a nuclear retention element.

33. The genome editing system of claim 1, wherein the payload RNA further comprises a Cas9 or Cas12 guide RNA, and wherein the Cas9 or Cas12 guide RNA comprises an extension with a 5′ homology sequence, a 3′ homology sequence, a 5′ untranslated region (UTR), a 3′ UTR, an insertion template, or any combination thereof.

34. (canceled)

35. The genome editing system of claim 1, wherein the R2 element enzyme comprises a nuclear localization signal (NLS).

36. The genome editing system of claim 1, wherein the insertion template comprises a template for a reporter gene, a transcription factor gene, a transgene, an enzyme gene, or a therapeutic gene.

37. A method of inserting a large nucleic acid into a genome within a cell using a Cas9 or Cas12 fusion protein, wherein the method comprises supplying a Cas9 or Cas12 fusion protein to a cell, wherein the Cas9 or Cas12 fusion protein is supplied with a payload RNA template, wherein the RNA template is reverse transcribed by the Cas9 or Cas12 fusion protein prior to being inserted into the genome of the cell; and wherein the large nucleic acid is inserted into the genome of the cell.

38. (canceled)

39. (canceled)

40. (canceled)

41. A method of inserting an exogenous nucleic acid into the genome of a post-mitotic cell, wherein the method comprises subjecting the genome of the post-mitotic cell to a modified Cas9 protein that inserts the exogenous nucleic acid into the genome of the post-mitotic cell.

42. (canceled)

43. (canceled)

44. (canceled)

45. (canceled)

46. (canceled)

47. A genome editing system comprising:
i) a payload RNA,
wherein the payload RNA comprises an insertion template and optionally one or more of a 5′ homology region, a 3′ homology region, and a protein binding element,
wherein the insertion template comprises a sequence for a nucleic acid insertion into the genome:
ii) a non-LTR site specific retrotransposon element enzyme;
wherein the non-LTR site specific retrotransposon element enzyme comprises a reverse transcriptase domain and, optionally, a nuclease or nickase domain, and
wherein if the non-LTR-site specific retrotransposon element enzyme does not comprise the optional nuclease or nickase domain, the genome editing system further comprises
iii) a nuclease or nickase enzyme.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. A method of inserting a large nucleic acid into a genome within a cell using a Cas9 or Cas12 fusion protein, wherein the method comprises supplying a Cas9 or Cas12 fusion protein to a cell, wherein the Cas9 or Cas12 fusion protein is supplied with a payload RNA template, wherein the RNA template is reverse transcribed by the Cas9 or Cas12 fusion protein prior to being inserted into the genome of the cell; and wherein the large nucleic acid is inserted into the genome of the cell.

92. (canceled)

93. (canceled)

94. (canceled)

95. A method of inserting an exogenous nucleic acid into the genome of a post-mitotic cell, wherein the method comprises subjecting the genome of the post-mitotic cell to a modified Cas9 protein that inserts the exogenous nucleic acid into the genome of the post-mitotic cell.

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)
